(19) United States
(12) Patent Application Publication
(10) Pub. No.: US 2011/0160266 A1
(43) Pub. Date: Jun. 30, 2011

(54) Novel Anti-Arrhythmia Agent

(75) Inventor: Paul Wolkowicz, Hoover, AL (US)

(73) Assignee: THE UAB RESEARCH FOUNDATION, Birmingham, AL (US)

(21) Appl. No.: 13/061,125

(22) PCT Filed: Aug. 31, 2009
(86) PCT No.: PCT/US2009/055546

§ 371 Date: Feb. 25, 2011

Related U.S. Application Data
(60) Provisional application No. 61/093,261, filed on Aug. 29, 2008.

(51) Int. Cl. A61K 31/415; A61P 9/06

(52) U.S. Cl. 514/399

(57) ABSTRACT

It has been discovered that SKF-96365 prevents and reverses ectopic activity in myocyte cells, and that SKF-96365 is effective against all types of ectopic activity. Methods are provided using SKF-96365 and its derivatives to prevent and reverse electromechanical disorders of myocytes; these include methods of treatment and prevention of cardiac arrhythmias in human and animal subjects. These further include methods of preventing and reversing arrhythmias in cardiac muscles and hearts. These further include methods of preventing and reversing spontaneous mechanical activity in a myocyte. Pharmaceutical drugs for the treatment and prevention of cardiac arrhythmia are described, based on SKF-96365. Kits for employing the methods are also described.

### CROSS REFERENCE TO RELATED APPLICATIONS

The present disclosure claims the benefit of U.S. Provisional Application No. 61/093,261, filed Aug. 29, 2009.

### FIELD OF THE DISCLOSURE

The present disclosure relates generally to agents that affect the activity of cardiac muscle. More particularly, the present disclosure relates to novel agents that inhibit cardiac arrhythmia. Methods of using the disclosed agents, pharmaceuticals comprising the agents, kits comprising the agents and kits for the practice of the disclosed methods are provided.

### BACKGROUND

Atrial and ventricular arrhythmias are abnormal cardiac electromechanical activities that occur independently of normal rhythmic heart function. Some types of arrhythmias that are known to occur (in increasing order of severity) are single ectopic beats (pre-mature contractions), rapid prolonged ectopic activity (tachycardia), and “disorganized” rapid ectopic activity (fibrillation). Cardiac arrhythmias are a leading cause of death and disability in industrialized countries, a leading cause of premature death, and a major health care cost. They are a major clinical burden and account for one-fourth to one-third of all premature deaths. The frequency of many arrhythmias increases with age and so they will increase their burden on the medical system as the average age of the U.S. population continues to increase.

Arrhythmia in the upper heart chambers (the atria) predisposes to stroke, exacerbates ventricular failure, is increasingly common with age, and is refractory to most non-invasive therapeutic approaches. Arrhythmia in the lower heart chambers (the ventricles) increases in frequency following myocardial infarction and during heart failure, and causes “sudden death,” a common cause of premature mortality.

The heart contains three types of cells that effect its primary physiological purpose, rhythmic contractions that propel blood through the circulatory system: (1) cells that spontaneously generate recurrent electrical signals, a property known as normal automaticity, (2) cells that conduct these signals throughout the heart, and (3) cells known as myocytes that convert the electrical signals into a contractile event.

Normal myocyte rhythmic contraction consists of two general phases (FIG. 1). The first is the excitation phase wherein an external electrical stimulus provokes the opening of myocyte plasma membrane sodium channels. Sodium entry depolarizes the myocyte which allows voltage-dependent calcium entry to occur during the subsequent action potential repolarization. Myocyte contraction then occurs in the second phase because of a process known as calcium-induced calcium release. Here the small amount of calcium that enters myocytes during repolarization provokes the release of large amounts of calcium from the sarcoplasmic reticulum (SR), the main myocyte calcium store. SR calcium release occurs via the ryanodine receptor (RyR) calcium release channel. The resultant increase in myocyte cytoplasmic calcium activates the troponin C-linked actomyosin system to affect contraction. The SR calcium ATPase (SERCA) protein then transports cytosolic calcium back into the SR lumen, producing muscle relaxation, restoring the SR calcium store, and readying the muscle for the next wave of external stimulation.

The accepted model for excitation contraction coupling holds that an initial depolarization of myocytes opens sarcolemmal sodium channels to produce myocyte depolarization (FIG. 1, Inset; AP, left upstroke). Myocytes then repolarize and during that time their voltage-dependent calcium channels open allowing a small amount of trigger calcium to enter cells (FIG. 1; arrow @ I_{Ca}). This trigger calcium binds to the sarcoplasmic reticulum (SR) RyR and effects calcium-induced release of SR calcium (FIG. 1, Inset [Cai]). This cytosolic calcium binds to the troponin C complex on myofilaments effecting muscle contraction (FIG. 1, Myofilaments and Inset, Contraction). Removal of calcium from the cytosol via the SR calcium ATPase (FIG. 1, ATP) lowers cytosolic calcium and produces muscle relaxation. Resting equilibrium restored, the myocyte awaits another wave of depolarization from the sino-atrial node or from a pacing stimulator.

FIG. 2 provides a graphical representation of this process. In FIG. 2 myocytes maintain both (1) a resting potential of −70 to −85 mV (negative inside) across their plasma membrane and (2) a ˜10,000-fold gradient of calcium from the outside (˜2 mM) to the myocyte cytoplasm (˜0.00001 mM). Following (3) myocyte excitation (i.e., plasma membrane depolarization), small amounts of extracellular calcium enter the myocyte which (4) trigger the release of calcium from intra-myocyte calcium stores sequestered in the SR. Calcium exits from the SR through the RyR. (5) This released calcium then activates myocyte actin-myosin complexes, producing muscle contraction. (6) Cytoplasmic calcium is subsequently transported back into the SR lumen via the SR calcium ATPase (SERCA) to await another wave of depolarization.

Myocytes do not normally generate electrical or mechanical activity spontaneously as ‘automatic’ sinoatrial cells do. Rather, myocytes require an external electrical stimulus to initiate contraction, which is their fundamental physiological role. A clear example showing heart excitability but non-automaticity is presented in FIG. 3A. Here an isolated, superfused rat left atrial appendage contracts only under the influence of a 1 Hz pacing stimulus (1 Hz). When the stimulus is terminated (Rest), this muscle becomes quiescent.

Arrhythmias are disruptions in this normal pattern of excitation and contraction. Arrhythmias arise in all three groups of cells but the most medically important ones are those that that occur when myocytes generate action potentials or depolarizations that either require or occur independently of an external depolarizing stimulus. These ectopic action potentials or depolarizations initiate SR calcium release followed by abnormal heart contraction. Arrhythmic events that require an external stimulus are triggered activity while those that do not are termed automatic events like tachycardias. Both arise from disrupted myocyte calcium homeostasis. These ectopic action potentials or depolarizations initiate SR calcium release followed by abnormal heart contraction.

Because most arrhythmias result from altered intra-myocyte calcium handling, pharmaceuticals aimed at managing the action potential may not affect the primary cellular cause of the arrhythmic event. Indeed few classes of pharmaceuticals effectively prevent or reverse atrial or ventricular arrhythmic activity and these generally act by modulating the myocardial action potential. However, clinical studies have shown that these anti-arrhythmic agents themselves can be pro-arrhythmic, increasing patient mortality and limiting their effectiveness as anti-arrhythmic therapies. Thus, few pharmaceuticals effectively prevent or reverse clinically relevant forms of arrhythmias without significant side-effects on the normal electrical activity of the heart. One likely reason for this paucity of effective anti-arrhythmic pharmaceuticals may be that agents which target the myocyte proteins or the myocyte processes that underlie arrhythmic activity have not yet been identified.

The prior art is lacking in compounds and methods to effectively treat and/or prevent cardiac arrhythmias, despite a long-felt need for such compounds and methods. Despite the seriousness of the disease, and the mortality and morbidity associated therewith, the prior art has failed to develop and implement consistent treatments for therapeutic intervention.

Without limiting the description and claimed embodiments, the present disclosure demonstrates that the small molecule SKF-96365 and derivatives thereof act to inhibit all forms of ectopic activity through a previously unknown pathway, creating previously unsuspected potential medical applications. Therefore, the present disclosure addresses these long standing problems in the art.

### SUMMARY

The disclosure provides SKF-96365 and derivatives thereof as novel anti-arrhythmic agents, kits comprising the disclosed novel anti-arrhythmic agents and pharmaceuticals and medicaments comprising the novel anti-arrhythmic agents. The disclosure further provides methods of treating and/or preventing arrhythmia. The disclosure further provides methods of inhibiting spontaneous mechanical activity (SMA) in a myocyte. The disclosure further provides methods of inhibiting arrhythmia in a cardiac muscle.

It is an objective of some embodiments of the processes, machines, manufactures, compositions of matter, and other teachings of the present disclosure to provide a pharmaceutical composition for the treatment or prevention of an arrhythmia comprising one of the disclosed compounds. It is an objective of some embodiments of the processes, machines, manufactures, compositions of matter, and other teachings of the present disclosure to provide a method of treating or preventing an arrhythmia in a subject in need thereof comprising administering to the subject a pharmaceutical composition comprising one of the disclosed compounds. It is an objective of some embodiments of the processes, machines, manufactures, compositions of matter, and other teachings of the present disclosure to provide a method of preventing or reversing arrhythmia in a cardiac muscle comprising contacting the muscle with one of the disclosed compounds. It is an objective of some embodiments of the processes, machines, manufactures, compositions of matter, and other teachings of the present disclosure to provide a method of inhibiting SMA in a myocyte, comprising contacting the myocyte with one of the disclosed compounds. It is an objective of some embodiments of the processes, machines, manufactures, compositions of matter, and other teachings of the present disclosure to provide a kit for the treatment or prevention of arrhythmia comprising a dosage of a pharmaceutical composition comprising one of the disclosed compounds.

### BRIEF DESCRIPTION OF THE DRAWINGS

FIG. 1: Model for myocardial electrical-mechanical coupling: The accepted model for excitation contraction coupling holds that an initial depolarization of myocytes opens sarcolemmal sodium channels to produce myocyte depolarization (inset; AP, left upstroke). Myocytes then repolarize and during that time their voltage-dependent calcium channels open allowing a small amount of trigger calcium to enter cells (diagram; arrow adjacent to I_{Ca}). This trigger calcium binds to the sarcoplasmic reticulum (SR) ryanodine receptor (RyR) and effects calcium-induced release of SR calcium (inset, lined marked [Ca]ᵢ). This cytosolic calcium binds to the troponin C complex on myofilaments (diagram) effecting muscle contraction (inset; line marked “contraction”). Removal of calcium from the cytosol via the SR calcium ATPase (diagram; SR surface component marked “ATP”) lowers cytosolic calcium and produces muscle relaxation. When resting equilibrium is restored, the myocyte awaits another wave of depolarization from the sino-atrial node or from a pacing stimulator.

FIG. 2: Normal myocyte excitation-contraction coupling. Schema shows a myocyte with (1) a resting membrane potential of about −80 mV and (2) a 10.000-fold calcium gradient. (3) The arrival of a wave of excitation originating from the sino-atrial node (SAN) allows a small amount of calcium entry via the L-type calcium channel (LTCC) which (4) induces large amounts of calcium release from the sarcoplasmic reticulum (SR). (5) Released calcium triggers contraction while (6) subsequent calcium reuptake into the SR by the SERCA produces relaxation.

FIG. 3: Experimental examples of (A) normal myocardial excitation-contraction coupling, (B) triggered activity and (C) automatic activity. Rat left atria were isolated and superfused in Krebs-Henseliet buffer at 30° C. A. These normal left atria produce mechanical force when exposed to an external one hertz pacing stimulus (1 Hz). These muscles do not contract in the absence of stimulation (Rest). Thus normal left atria are not automatic. B. Left atria exposed to ATX II exhibit triggered mechanical events. That is in the face of a 1 Hz pacing stimulus multiple mechanical events (e.g., ‡) are elicited following a single stimulus. These ectopic events arise because of afterdepolarizations and are triggered events because they require a previous depolarization to occur; that is, no ectopic activity arises in the absence of pacing (Rest). C. Normal heart muscle can also generate automatic activity. Left atria treated with 2-aminoethoxydiphenyl borate (2-APB) produce repeated, spontaneous contractile events in the absence of a pacing stimulus (Rest; *). Thus normal non-automatic myocardium can produce both triggered and automatic activity.

FIG. 4: Examples of early afterdepolarizations and delayed afterdepolarizations. A. Early afterdepolarizations (EADs) arise during the repolarization phase of the action potential (Phases 2 and 3). If EADs are of a sufficient magnitude, they will trigger an ectopic mechanical event. B. Delayed afterdepolarizations (DADs) arise when the myocyte has returned to its resting potential. Similar to EADs, if a DAD can raise the resting potential of a myocyte to a value great enough to open sodium channels then the myocyte will initiate an ectopic action potential that produces an attendant ectopic mechanical event. These two types of afterdepolarizations are responsible for triggered arrhythmic activity.

FIG. 5: ATX II induction of early afterdepolarizations. ATX II binds to the sodium channel and increases the late sodium current. As a result, ATX II (i) markedly prolongs the action potential duration and (ii) increases myocyte sodium content which (iii) effects myocyte calcium loading via the sodium-calcium exchanger. As a result of this sequence of events, myocytes treated with ATX II produce EADs which produce triggered mechanical activity.

FIG. 6: Parent compound SKF-96365: Molecular structure of parent anti-arrhythmic claimed in this disclosure, SKF-96365.

FIG. 7: Increasing superfusate potassium reverses SMA. Upper panel Mechanical function typical of 0.1 Hz paced atrial appendage superfused in KH and exposed to 15 μM 2-APB (2APB). Following the appearance of SMA, increasing amounts of KCl were added to the superfusate (lines marked “KCl” and associated millimolar concentrations). Lower panel (▪) The fraction of the total preparations (n=7 total preparations) exhibiting prolonged SMA relative to KH buffer at 5.8 mM KCl (all preparations exhibited SMA at 5.8 mM); (∘) basal force of contraction measured in untreated atria (n=7 total) exposed to increasing KCl relative to the basal force measured in KH buffer at 5.8 mM KCl (defined as the “initial force”).

FIG. 8: 2-APB induces SMEs in isolated, superfused rat left atrial appendage. A: Left panels; Mechanical function typical of isolated, superfused rat left atrial appendage paced at 3 Hz. Right panels; Mechanical function typical of 3 Hz paced appendage exposed to 15 μM 2-APB for 15 min. B: The number of spontaneous mechanical events (SME), defined as spontaneous increases in force (upper right panel: *), that occurred per min in muscles superfused in KH, paced at 3 Hz, and exposed to 2-APB at the following concentrations: 0 (n=5), 2 (n=5) 7.5 (n=8), 15 (n=10), or 22 μM (n=8). Exposure time was constant at 15 min. Mean±SEM are reported. *=p<0.05 vs. 0 μM 2-APB; ‡=p<0.05 vs. 15 μM 2-APB.

FIG. 9: 2-APB induces spontaneous mechanical activity with prolonged diastolic interval. A: Mechanical function typical of 0.1 Hz paced atrial appendage (n=6). B: Mechanical function typical of 0.1 Hz paced muscle (n=8) exposed to 7.5 μM 2-APB for 15 min. One or more SMEs (*) occurred in each muscle. C: Mechanical function typical of 0.1 Hz paced appendage (n=8) exposed to 22 μM 2-APB for 15 min. Isolated SMEs were followed by prolonged spontaneous mechanical activity (SMA; right part of panel).

FIG. 10. A: Decreasing superfusate sodium prevents SMA. Upper panel: Mechanical function typical of 0.1 Hz paced left atrial appendage washed with 250 ml of KH containing 109 mM sodium and constant chloride of 126 mM (line marked 109 mM Na). Fifteen minutes after washing, this muscle was exposed to 15 μM 2-APB (line marked 2APB) for 15 min. Lower panel: The fraction of total muscle preparations exhibiting SMA (e.g. FIG. 3C, Rest) at each concentration of superfusate sodium (n=6-8 total preparations) relative to KH buffer at 145 mM Na (all preparations in 145 mM KH buffer exhibited SMA). No error bars are reported as these experiments tested whether preparations did or did not exhibit SMA. B: Decreasing superfusate chloride prevents SMA. Upper panel: Mechanical function of 0.1 Hz paced left atrial appendage washed with 250 ml of KH containing 30 mM total chloride and a constant sodium of 145 mM (line marked “30 mM Cl”). Fifteen minutes after washing, this atrium was exposed to 15 μM 2-APB (2APB) for 15 min. Lower panel: The number of muscle preparations exhibiting SMA (e.g., FIG. 3C, Rest) at each concentration of superfusate Cl (n=6-8 total preparations) relative to KH buffer at 126 mM Cl (all preparations exhibited SMA in KH buffer at 126 mM Cl).

FIG. 11: DIDS reverses SMA. Upper panel: Mechanical function of 0.1 Hz paced atrial appendage superfused in KH and exposed to 15 μM 2-APB (2APB). Following the appearance of SMA it was titrated with 100 to 300 μM DIDS (lines marked DIDS underlain by micromolar concentrations). Lower panel: The percentage of the total muscle preparations (n=7 total preparations) exhibiting prolonged SMA after a 3-5 min incubation at each concentration of DIDS relative to KH at 0 μM DIDS (all preparations exhibited SMA in ICH buffer at 0 μM DIDS).

FIG. 12: 2-APB significantly decreases the maximum force of atrial contraction under conditions that suppress SMA. −2APB: KH; Maximum forces of atrial contraction (PRP) (n=6) were obtained from a 1 min PRP performed at the end of a 35 min superfusion. −2APB ΔNa:DIDS; Maximum forces of atrial contraction were measured 23 min after lowering superfusate sodium to 82 mM (ΔNa; n=8) or adding 400 μM DIDS (DIDS; n=8). +2APB: ΔNa:DIDS; Maximum forces of atrial contraction were measured in preparations exposed to 22 μM 2-APB 23 min after lowering superfusate sodium (ANa; n=8) or adding DIDS (DIDS; n=8). These values were compared to PRPs performed ˜10 min prior to the addition of 2-APB. Mean±SEM. *=p<0.05 versus (−)-2-APB. ‡=p<0.05 versus ΔNa.

FIG. 13: 2-APB induces left atrial appendage SMEs in the absence of electrical stimulation. A: Mechanical function typical of 3 Hz paced atria (n=5) (3 Hz stimulus) subjected to 5 min of rest (line marked “rest”). Potentiation of mechanical force (*) occurred with re-initiation of pacing. B: Mechanical function typical of 3 Hz paced atria (n=6) (line marked “3 Hz stimulus”) exposed to 15 μM 2-APB for 10 min (line marked “2APB”) followed by a 5 min rest. SMEs (§) occurred in all 2-APB-treated atria. Post-rest potentiation (*) was blunted in all 2-APB-treated.

FIG. 14: Induction of sporadic and tachycardic automatic ectopy in isolated left atrial appendage. (A) Mechanical function of an isolated rat left atrial appendage paced at 0.1 Hz and superfused at 30° C. In the absence of pacing (line marked “rest,” magnified at inset) this muscle is quiescent. (B) A second left atrial appendage superfused and paced as in (A). This muscle was exposed to 22 μM 2-APB where indicated. After a few minutes this muscle produces mechanical events that occur independently of external stimulation. In the absence of pacing (line marked “rest,” magnified at inset) this muscle produces persistent SMA. (C) Muscle identical to (B) except that it was treated with 30 nM isoproterenol (line marked “Iso”). Under this condition isolated appendages produce spontaneous activity in the absence of pacing (line marked “rest,” magnified at inset) at a rate of ˜230 contractions/min. This spontaneous tachycardic activity (STA) exceeds the rate of normal right atrial pacemaker-driven function, ˜170 contractions/min at 30° C.

FIG. 15: Ranolazine blocks ectopic activity in this bio-assay. (A) Titration curve of ranolazine reversal of SMA in superfused left atrial appendage. Superfused appendages (n=7) were treated with 2-APB and following the appearance of SMA they were titrated with increasing concentrations of ranolazine for 3-5 min at any concentration. Rates of SMA in the absence of pacing were recorded. (B) Typical raw mechanical data for an appendage treated with 2-APB and then with 0, 10 or 80 μM ranolazine. All data are in the absence of pacing.

FIG. 16: Flecainide suppression of SM. (A) Upper panel: Left atrial appendage paced at 0.1 Hz. Middle panel: Same appendage superfused with 22 μM 2-APB; SMA occurs in this muscle. Lower panel: Appendage treated with 65 μM flecainide following the appearance of SMA. (B) Summary of data for left atrial appendages (n=8) that were exposed to 22 μM 2-APB and, following the appearance of SMA, were titrated with increasing concentrations of flecainide. The % of preparations exhibiting SMA in the absence of pacing (SCA) was recorded after a 5 min exposure to any concentration of flecainide.

FIG. 17: Lowering superfusate sodium reverses STA. A. The mechanical function of an unpaced left atrial appendage (n=9) undergoing STA in the presence of 300 nM Bay K 8644 and 20 μM 2-APB. Superfusate sodium was reduced from 145 to 82 mM where indicated. B. The mechanical function of the unpaced left atrial appendage in (A) exposed to BayK 8644, 2-APB, and 82 mM sodium. 0.1 Hz pacing was reinstituted where indicated (0.1 Hz)

FIG. 18: Rat left atria contain HCN2 and HCN4 cDNAs. Total RNA was extracted from 3 rat right atria and 3 rat left atria. RT-PCR analyses for HCN1-4 were performed as described in Methods. Bar graphs summarize these amplifications relative to a cyclophilin control. White bars marked “RA” represent right atria; shaded bars marked “LA” represent left atria. Data are mean±S.E.M.

FIG. 19: (A) Concentration dependence of zatebradine suppression of STA. Nine 3 Hz-paced left atrial appendages (∘) were exposed to 2-APB and BayK 8644. After the appearance of tachycardia, appendages were titrated with 0 to 100 μM zatebradine and its effect on the frequency of STA was recorded 3-5 min after any addition. Nine rat right atria (▪) were titrated with 0 to 100 μM zatebradine. The effect of zatebradine on right atrial contraction frequency was recorded. Percent of the initial rate of spontaneous tachycardic activity (left atria) or spontaneous contraction (right atria) are reported. (B) ZD-7288 suppresses STA. Seven 3 Hz-paced left atrial appendages (∘) were exposed to 2-APB and BayK 8644. After the appearance of STA, muscles were titrated with 0 to 100 μM ZD-7288 and its effect on STA was recorded 3-5 min after any addition. Seven rat right atria (▪) were titrated with 0 to 100 μM ZD-7288. The effect of ZD-7288 on right atrial contraction frequency was recorded. % of the initial rate of spontaneous tachycardia (left atria) or spontaneous contraction (right atria) are reported. All data are mean±S.E.M.

FIG. 20: Zatebradine decreases the frequency of STA. A. The mechanical function of a 3 Hz-paced rat left atrial appendage (n=9) subjected to ˜15 sec of rest. B. The mechanical function of a 3 Hz-paced appendage (n=9) exposed to 20 μM 2-APB (2-APB) for 10 min prior to rest. C. The mechanical function of a 3 Hz-paced appendage (n=9) treated with 300 nM (−) BayK 8644 for 5 min and 2-APB for 10 min. Discordant mechanical events occur with 3 Hz pacing (‡) D. The mechanical function of a 3 Hz-paced appendage (n=9) treated with BayK 8644 and 2-APB for 10 min and then with 70 μM zatebradine (ZTB) for 10 min.

FIG. 21: Induction of sporadic, tachycardic, and chaotic ectopy in rat right ventricular muscle strips. A. Mechanical function of right ventricular muscle strip superfused at 30° C. and paced at 0.5 Hz. B. Similar superfused right ventricular muscle strip exposed for ˜10 min to 22 μM 2-APB. SMA occurs in this muscle (‡). C. Right ventricular muscle strip treated with 30 nM isoproterenol and 2-APB show STA in the absence of a 1 Hz pacing stimulus (line marked “rest”). Ventricular muscle treated in this way shows disorganized mechanical activity in the presence of pacing (e.g. §).

FIG. 22: Temperature dependence of left atrial appendage tachycardia and right atrial mechanical activity. Rat left atrial appendage (▪; n=6-8) superfused at 30° C., paced at 0.1 Hz, treated for 5 min with 300 nM (−)BayK 8644 and then for 10 min with 22 μM 2-APB. Pacing was stopped and rates of spontaneous mechanical activity (SMA) were recorded. A second group of appendages (n=8) were superfused at 23° C. and treated identically to the first. 10 min later SMA was measured without pacing and muscle bath temperature was increase to 37° C. Maximum rates of SMA were measured over 10 min. Rat right atria (Δ; n=8) were superfused at 30° C. and rates of pacemaker-driven mechanical contraction were recorded. A second group was superfused at 23° C. and their contraction rates were recorded; muscle bath temperature was increased to 37° C. and maximum contraction rates were measured over 10 min.

FIG. 23: Upper: Mechanical function of a superfused rat right atrium measured without pacing at 37° C. Middle: Mechanical function of a 0.1 Hz-paced, superfused left atrial appendage treated for 5 min with 300 nM (−)BayK 8644 alone at 37° C. Function is measured here without pacing. Lower: Mechanical function of a rat left atrial appendage treated as per the second group in (A) and measured without pacing.

FIG. 24: Induction of chaotic ectopy in isolated left atrial appendage and right ventricular muscle strips. A. Mechanical function of a rat left atrial appendage superfused at 37° C., paced at 5 Hz, and exposed to 22 μM 2-APB and 300 nM BayK 8644 (line marked “BayK”). All muscles exhibit chaotic, disorganized mechanical activity (§) when paced at this physiological rate. In the absence of pacing (line marked “Rest”) muscles show only STA. B. Mechanical function of a rat right ventricular muscle strip superfused at 30° C., paced at 3 Hz, and exposed to 22 μM 2-APB and 30 nM isoproterenol (line marked “Isoprel”). All such muscles exhibit chaotic mechanical function (§) in the presence of pacing. In the absence of pacing (line marked “Rest”) these muscle show only STA.

FIG. 25: Effect of SKF-96365 and verapamil on left atrial triggered activity. It was tested whether the canonical store-operated calcium channel inhibitor SKF-96365 or the canonical voltage-dependent calcium channel blocker verapamil could suppress triggered ectopic contractions induced by ATX II. 1 Hz-paced left atria (n=5-7) were treated with 25 nM ATX II and following the appearance of triggered activity were titrated with increasing concentrations of SKF-96365 or verapamil. Ectopic contractions then were recorded after ˜5 min incubation at each concentration. SKF-96365 suppressed ectopic contractions with an IC₅₀ of ˜12 μM while verapamil did not affect the rate of triggered activity in these left atria.

FIG. 26: Summary of the effect of SKF-96365 and verapamil on left atrial force of contraction: 1 Hz-paced left atria (n=5-7) were treated with 25 nM ATX II and following the appearance of triggered activity were titrated with increasing concentrations of SKF-96365 or verapamil. Force of contraction was recorded after ˜5 min incubation at each concentration. SKF-96365 did not affect left atrial force of contraction while verapamil markedly decreased contractile force most likely because of its ability to block slow calcium channel activity and calcium-induced calcium release.

FIG. 27: SKF-96365 blockade of triggered activity. A. The normal contraction pattern of a left atrium paced at 1 Hz. This non-automatic muscle contracts only when stimulated. B. The same muscle exposed to 25 nM ATX II shows triggered activity as contractile doublets following a single electrical stimulus. C. The same ATX II-treated left atrium following a ˜5 min exposure to 20 μM SKF-96365. This muscle shows no triggered events following 1 Hz pacing stimulation. Thus SKF-96365 suppresses triggered activity.

FIG. 28: SKF-96365 reversal of triggered activity. A. Normal left atria contract only when stimulated with a 1 Hz pacing stimulus. B. Left atria exposed to ATX II show triggered activity (*). C. A left atrium treated with ATX II shows triggered activity as in (B); adding SKF-96365 to the superfusate suppresses triggered activity. That is, left atria treated with ATX II and then SKF-96365 produce only single contractile events following stimulation.

FIG. 29: Verapamil does not prevent triggered activity. A. A normal left atrium paced at 1 Hz shows mechanical function only with stimulation. B. Left atrium treated with ATX II shows triggered activity as doublets of contraction. C. Left atrium exposed to ATX II and then to verapamil shows markedly depressed mechanical function. However, if the scale of this tracing is expanded (inset), the continued presence of triggered events is clearly seen. Thus verapamil can suppress mechanical function but not triggered activity.

FIG. 30: SKF-96365 blockade of triggered activity. Left atria were pre-treated with 20-25 μM SKF-96365 for ˜10 min prior to the addition of ATX II. Following the addition of ATX II, an inotropic response occurs most likely because of calcium loading and increased SR calcium content. However, despite the presence of ATX II, triggered activity does not occur in SKF-98365-treated muscle as it responds to the external pacing stimulus with a single contractile event and not a triggered pattern of doublets.

FIG. 31: SKF-96365 reversal of SMA. A superfused left atrium was treated with 2-APB. As expected the muscle exhibited sporadic mechanical events (SMEs) in the absence of pacing (line marked “rest”). Exposing this left atrium to 25 μM SKF-96365 suppressed SMA within 2-3 min and the muscle became quiescent in the absence of pacing. SKF-96365 did not affect normal excitation-contraction coupling as restoring the 0.1 Hz pacing stimulus provoked mechanical activity similar in magnitude to untreated left atrium (e.g., left of panel) which occurred only following a pacing stimulus.

FIG. 32: Dose-response curve of SKF-96365 reversal of sporadic abnormal automaticity. Rat left atria were paced at 0.1 Hz and exposed to 2-APB. Following the appearance of SMA these left atria were titrated with increasing concentrations of SKF-96365 for 3-5 min at any concentration and the rate of spontaneous contractions was recorded. SKF-96365 suppressed SMA.

FIG. 33: Summary of SKF-96365 blockade of STA. Rat left atria were exposed to 0 or to 50 μM SKF-96365 for 10 min. Following this pre-incubation, muscles were titrated with increasing concentrations of 2-APB and the rate of SMA was measured ˜10 min later in the absence of pacing. SKF-96365 prevents SMA.

FIG. 34: SKF-96365 reverses STA. To test whether SKF-96365 reverses tachycardic abnormal automaticity, a superfused left atrium was treated with Bay K 8644 to increase left atrial calcium and then with 2-APB. As expected the muscle began to exhibit STA in the absence of pacing (line marked “rest”). Exposing this muscle to SKF-96365 suppressed STA within 2-3 min and the muscle became quiescent in the absence of pacing. SKF-96365 did not affect normal excitation-contraction coupling as restoring the 0.1 Hz pacing stimulus provoked mechanical activity similar in magnitude to untreated left atrium (e.g., left of panel) which occurred only following a pacing stimulus.

FIG. 35: Dose-Response Curve of SKF-96365 reversal of tachycardic abnormal automaticity. Rat left atria (n=5-7) were paced at 0.1 Hz and exposed to Bay K 8644 and 2-APB. Following the appearance of STA these atria were titrated with increasing concentrations of SKF-96365 for 3-5 min at any concentration. The rates of spontaneous contractions then were recorded in the absence of pacing. SKF-96365 reverses STA under these conditions.

FIG. 36: SKF-96365 reverses chaotic, fibrillation-like mechanical activity in normal left atrium. Normal rat left atria were superfused at 37° C., paced at 6 Hz (a physiological rate under these conditions), exposed to BayK 8644 to load calcium and then to 2-APB to induce STA. Under these conditions muscle exhibited chaotic, fibrillation-like activity (upper left panel). These muscles then were exposed to ˜40 μM SKF-96365. After ˜5-8 min they no longer contracted chaotically but showed normal patterns of excitation-contraction coupling (upper right panel and lower panel) and required external stimulation for mechanical activity (bottom panel, line marked “rest”).

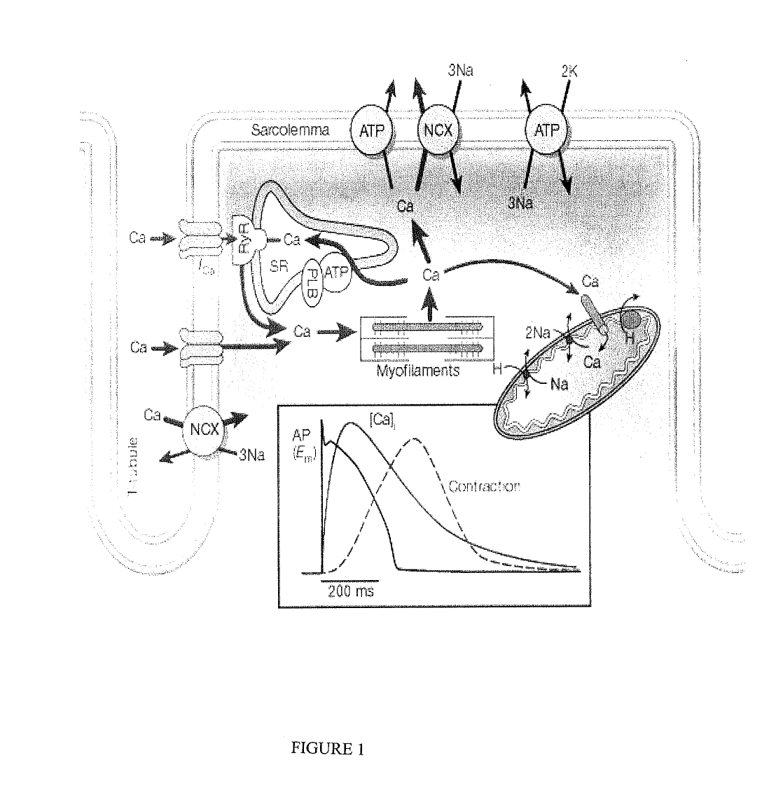

### DETAILED DESCRIPTION

Arrhythmias arise or are sustained through two mechanisms; (i) triggered activity and (ii) reentrant activity. The latter involves the abnormal propagation of electrical activity through the heart, and although reentrant activity is critical to sustaining arrhythmias, a triggering event generally precedes reentrant activity. Thus triggered activity is a major, perhaps the predominant, source of arrhythmia.

Triggered activity results from the abnormal generation of electrical activity in regions of the heart other than the SAN of the right atrium. The SAN is the origin of the spontaneous electrical activity that drives normal rhythmic atrial and ventricular contractions. Triggered activity occurs through two mechanisms afterdepolarizations and abnormal automaticity, and is thought to arise, at least in part, from altered myocytecalcium homeostasis, although other mechanisms may contribute as well.

Triggered activity involves the production of an abnormal action potential or depolarization in quiescent or repolarizing myocytes. In particular, a triggered arrhythmia occurs when a critical mass of resting myocytes spontaneously depolarize to produce a single wave or repeated waves of ectopic electrical activity that propagate through the heart and conflict with the normal rhythmic electrical activity generated by the SAN.

These ectopic triggered depolarizations arise from specific events within the myocyte. Briefly, it has been believed that triggered activity may occur as a result of aberrant calcium leakage from SR stores during the interval between normal, rhythmic myocyte excitation. Leaked SR calcium activates calcium-dependent electrogenic ion transporters in the myocyte plasma membrane. Efflux of leaked calcium via these electrogenic carriers is hypothesized to drive the resting myocyte membrane potential to more positive values until it reaches ˜−65 mV (↓Eₘ (resting membrane potential)). At this range of voltage, quiescent myocytes will spontaneously generate arrhythmic after-depolarizations and electrical activity (↑ADs (afterdepolarizations)).

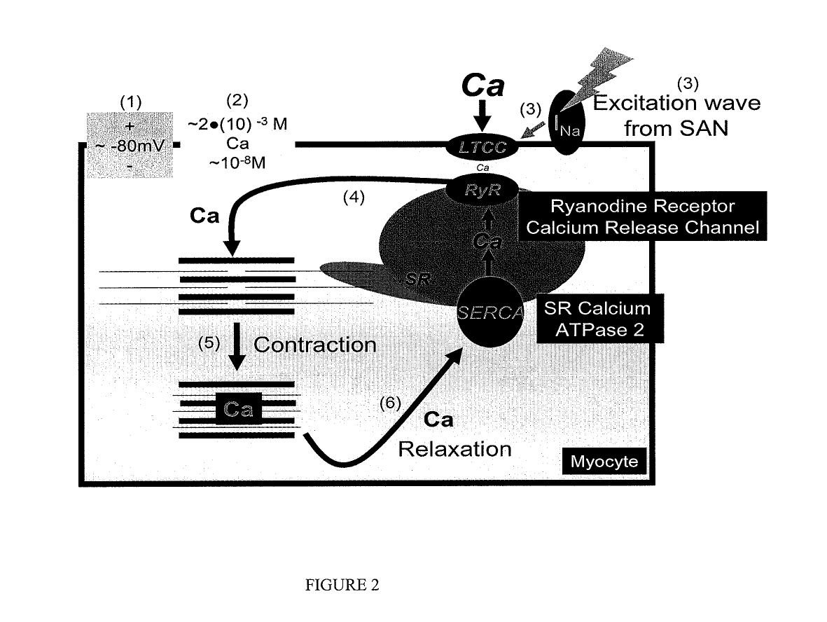

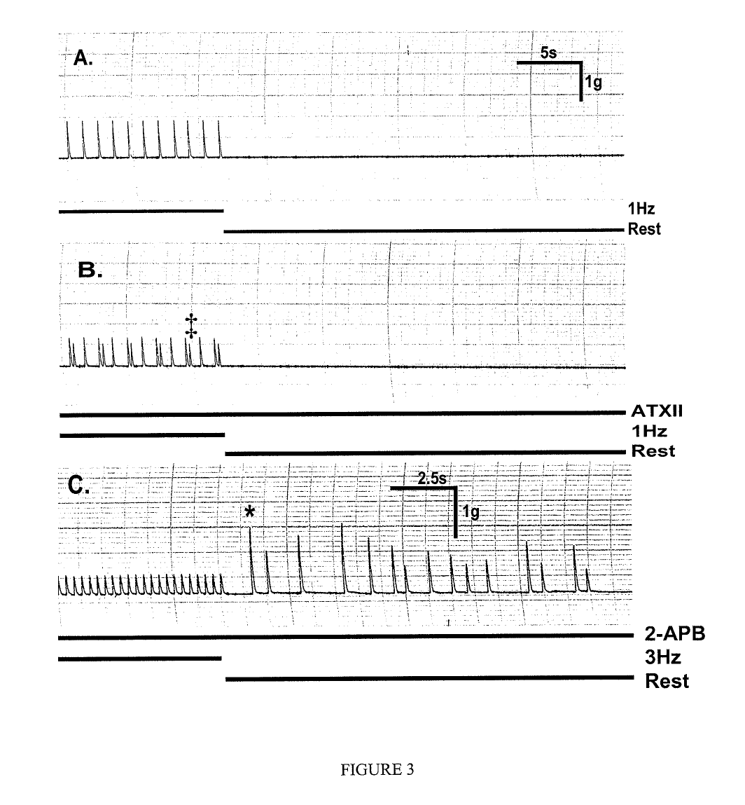

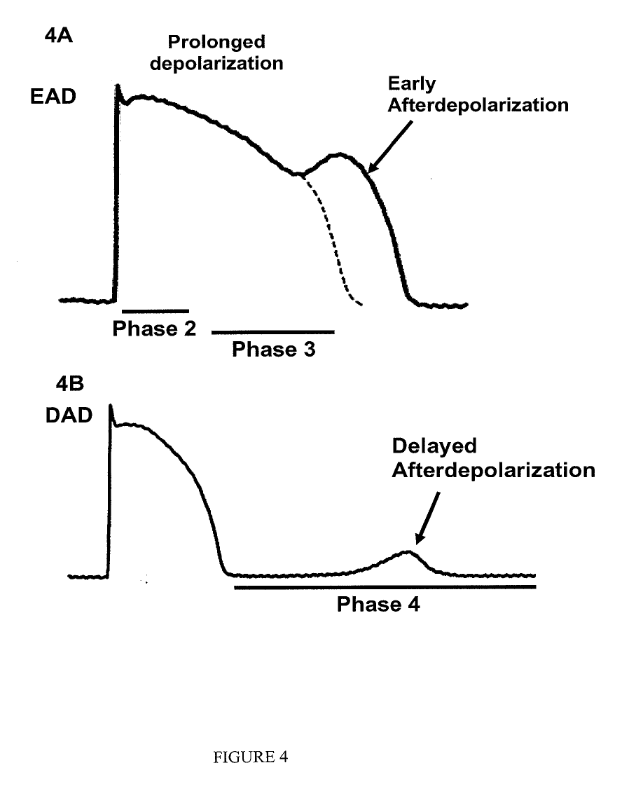

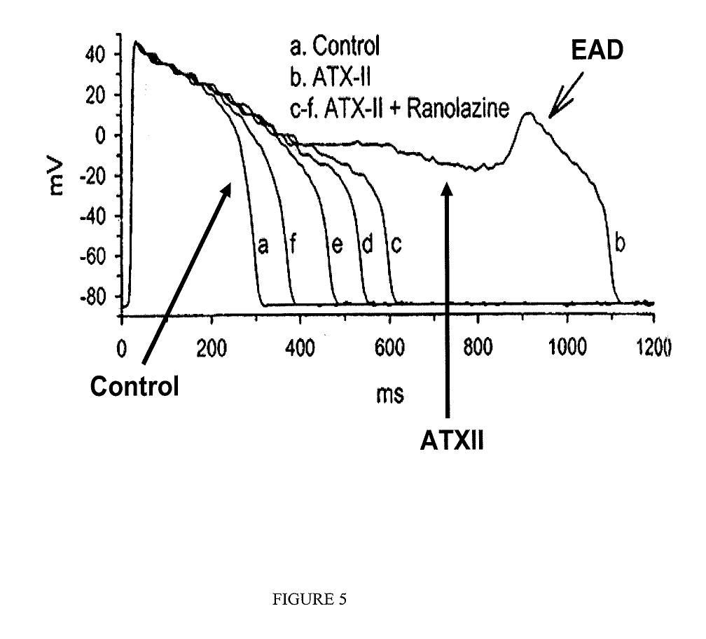

There are two types of afterdepolarizations; early afterdepolarizations (EADs) that occur early during repolarization and delayed afterdepolarizations (DADs) that occur in fully repolarized, resting myocytes (FIG. 4). Both EADs and DADs are defined as “triggered” activities as they require a preceding depolarization to occur. One model to elicit EADs is to expose heart muscle or myocytes to Anemonia sulcata Toxin II (ATX II). This 47 amino acid peptide specifically enhances late sodium current and prolongs the action potential duration. This leads to calcium loading of heart muscle followed by EADs during phase 2 or phase 3 of the action potential (FIG. 5). These EADs, if of sufficient magnitude, produce ectopic mechanical events (FIG. 2B; ‡). ATX II induces triggered activity as stopping the 1 Hz pacing stimulus in left atrial appendages stops both normal and ectopic mechanical activity (FIG. 3B; Rest).

Abnormal automaticity, another type of triggered arrhythmia, is characterized by rapid, repeated spontaneous depolarizations and contractions of non-automatic heart muscle. These events occur at a rate faster than the normal automaticity that is driven by the automatic cells of the SAN. Abnormal automaticity does not require prior external electrical stimulation. Prior to the current work, it was understood that this abnormal automaticity occurs through two mechanisms. The first is so-called re-entrant activity wherein an ectopic depolarizing impulse occurs in myocardium whose electrical properties affect the propagation of this impulse, or even a normal impulse, so as to generate a repeating electrical circuit imbedded in non-automatic heart muscle which then produces continuing, repeated depolarizations. The second relies on calcium leakage from ryanodine-sensitive SR calcium stores. Under appropriate conditions, conventional models suggests that this leakage can persistently provoke ectopic depolarizations at tachycardic rates.

### A. Definitions

The terms “prevention”, “prevent”, “preventing”, “suppression”, “suppress” and “suppressing” as used herein refer to a course of action (such as administering a compound or pharmaceutical composition of the present disclosure) initiated prior to the onset of a clinical manifestation of a disease state or condition so as to prevent or reduce such clinical manifestation of the disease state or condition. Such preventing and suppressing need not be absolute to be useful.

The terms “treatment,” “reverse,” “treat” and “treating” as used herein refers to a course of action (such as administering a compound or pharmaceutical composition) initiated after the onset of a clinical manifestation of a disease state or condition so as to eliminate or reduce such clinical manifestation of the disease state or condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method, compound or pharmaceutical composition of the disclosure.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a condition that is preventable by a method, compound or pharmaceutical composition of the disclosure.

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female.

The term “therapeutically effective amount” as used herein refers to an amount of a compound, either alone or as a part of a pharmaceutical composition, that is capable of having any detectable, positive effect on any symptom, aspect, or characteristics of a disease state or condition. Such effect need not be absolute to be beneficial.

The term “inhibits arrhythmia” as used herein refers to any property of a substance that tends to eliminate or reduce the likelihood, severity, or duration of arrhythmia in a cardiac muscle or a heart. The muscle or heart may be part of an intact animal or may be isolated from the animal. An agent that inhibits arrhythmia may do so in any context, including but not limited to a course of treatment or prevention. Such effect need not be absolute to be beneficial.

The term “arrhythmia” as used herein refers to spontaneous mechanical activities that occur independently of normal rhythmic heart function. Arrhythmia may occur in cardiac muscle, in any portion of a heart, or in an entire heart. Exemplary types of arrhythmia include, but are not limited to, triggered ectopic event, automatic ectopic events, sporadic automatic ectopic events, tachycardic automatic ectopic events and fibrillation-like chaotic activity.

The term “spontaneous mechanical activity” (abbreviated SMA) as used herein refers to mechanical events in a muscle or myocyte occurring in the absence of pacing stimulus or despite the presence of pacing stimulus.

The term “ectopic event” as used herein refers to a contractile event in a myocyte that occurs in the absence of or despite the presence of pacing stimulus.

The term “suppressor of spontaneous mechanical activity” as used herein refers to any agent that tends to arrest, abbreviate, curtail, inhibit, reduce in severity, reduce in likelihood, reduce in duration, prevent, or in any way improve spontaneous mechanical activity in a muscle or a myocyte. Such effect need not be absolute to be beneficial.

The term “effective inhibitory concentration” when used herein with regard to inhibitors of SMA refers to a concentration sufficient to reduce the likelihood, severity, or duration of SMA in a myocyte or a muscle. Depending on the application, the inhibition may take the form of the prevention of SMA, or, if the inhibitory agent is introduced to the muscle or myocyte after the onset of SMA, the inhibition may take the form of the reversal of SMA.

The term “atrium” or “atria” as used herein with regard to isolated cardiac muscle includes a left atrial appendage.

The term “halide” as used herein refers to a compound of a halogen with a more electropositive element or radical.

Generally, reference to a certain element such as hydrogen or H is meant to include all isotopes of that element. For example, if an R group is defined to include hydrogen or H, it also includes deuterium and tritium.

The phrase “unsubstituted alkyl” refers to alkyl groups that do not contain heteroatoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂ CH₃), —CH(CH₂CH₃)₂, —C(CH₃)₃, —C(CH₂CH₃)₃, —CH₂ CH(CH₃)₂, —CH₂ CH(CH₃)(CH₂ CH₃), —CH₂ CH(CH₂CH₃)₂, —CH₂ C(CH₃)₃, —CH₂ C(CH₂ CH₃)₃, —CH(CH₃)CH(CH₃)(CH₂CH₃), —CH₂CH₂CH(CH₃)₂, —CH₂CH₂CH(CH₃)(CH₂ CH₃), —CH₂CH₂CH(CH₂CH₃)₂, —CH₂CH₂C(CH₃)₃, —CH₂CH₂C(CH₂CH₃)₃, —CH(CH₃)CH₂ CH(CH₃)₂, —CH(CH₃)CH(CH₃)CH(CH₃)CH(CH₃)₂, and others. The phrase also includes cyclic alkyl groups such as cyclopropyl, cyclobutyl, cyclopentyl, cyclohexyl, cycloheptyl, and cyclooctyl and such rings substituted with straight and branched chain alkyl groups as defined above. The phrase also includes polycyclic alkyl groups such as, but not limited to, adamantyl, norbornyl, and bicyclo[2,2,2]octyl and such rings substituted with straight and branched chain alkyl groups as defined above. Thus, the phrase unsubstituted alkyl groups includes primary alkyl groups, secondary alkyl groups, and tertiary alkyl groups. Unsubstituted alkyl groups may be bonded to one or more carbon atom(s), oxygen atom(s), nitrogen atom(s), and/or sulfur atom(s) in the parent compound. In one embodiment, the unsubstituted alkyl groups include straight and branched chain alkyl groups and cyclic alkyl groups having 1 to 10 or 1 to 5 carbon atoms. In a specific embodiment, the unsubstituted alkyl groups include straight and branched chain alkyl groups having from 1, 2 or 3 carbon atoms.

The phrase “substituted alkyl” refers to an unsubstituted alkyl group as defined above in which one or more bonds to a carbon(s) or hydrogen(s) are replaced by a bond to at least one non-hydrogen and non-carbon atoms such as, but not limited to, a halogen atom in halides such as F, Cl, Br, and I; and oxygen atom in groups such as hydroxyl groups, alkoxy groups, carbonyl groups, carboxyl groups, aryloxy groups, aryloxy groups and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, enamines, hydrazones, and nitriles; a silicon atom in groups such as in trialkylsilyl groups, dialkylarylsilyl groups, alkyldiarylsilyl groups, and triarylsilyl groups; and other heteroatoms in various other groups. In certain embodiments, one or more non-carbon, non-hydrogen atom may be bonded to another non-carbon, non-hydrogen atom, provided that at least one non-carbon, non-hydrogen atom forms a bond to a carbon or hydrogen molecule of the alkyl group. The substituted alkyl group may be bonded to the parent molecule either through the alkyl portion or through the non-carbon/non-hydrogen group. Exemplary substituted alkyl groups include, but are not limited to, —COOH —SO(CH₂)ₘCH₃, CONH(CH₂)ₘCH₃, —N═N(CH₂)ₘCH₃, —N═NO(CH₂)ₘCH₃, N═NNH(CH₂)ₘCH₃, —(SO₄)(CH₂)ₘCH₃, —OSOO(CH₂)ₘCH₃, —OSiO(CH₂)ₘCH₃, —OCO(CH₂)ₘCH₃, —CO(CH₂)ₘCH₃, —SO₂(CH₂)_{M} CH₃, —S(CH₂)ₘCH₃, and —O(CH₂)ₘCH₃, wherein m is 0, 1, 2, 3, 4, 5, 6, 7, 8, 9, or 10. In one embodiment, an exemplary substituted alkyl is —OCH₃; the —OCH₃ group may be bonded to the parent molecule via the O molecule.

The phrase “unsubstituted alkenyl” refers to straight and branched chain and cyclic groups such as those described with respect to unsubstituted alkyl groups as defined above, except that at least one double bond exists between two carbon atoms.

The phrase “substituted alkenyl” has the same meaning with respect to unsubstituted alkenyl groups that substituted alkyl groups had with respect to unsubstituted alkyl groups. A substituted alkenyl group includes alkenyl groups in which a non-carbon or non-hydrogen atom is bonded to a carbon double bonded to another carbon and those in which one of the non-carbon or non-hydrogen atoms is bonded to a carbon not involved in a double bond to another carbon.

The phrase “unsubstituted alkynyl” refers to straight and branched chain groups such as those described with respect to unsubstituted alkyl groups as defined above, except that at least one triple bond exists between two carbon atoms.

The phrase “substituted alkynyl” has the same meaning with respect to unsubstituted alkynyl groups that substituted alkyl groups had with respect to unsubstituted alkyl groups. A substituted alkynyl group includes alkynyl groups in which a non-carbon or non-hydrogen atom is bonded to a carbon triple bonded to another carbon and those in which a non-carbon or non-hydrogen atom is bonded to a carbon not involved in a triple bond to another carbon.

The phrase “unsubstituted heterocyclyl” refers to both aromatic and nonaromatic ring compounds including monocyclic, bicyclic, and polycyclic ring compounds such as, but not limited to, quinuclidyl, containing 3 or more ring members of which one or more is a heteroatom such as, but not limited to, N, O, and S. Although the phrase “unsubstituted heterocyclyl” includes condensed heterocyclic rings such as benzimidazolyl, it does not include heterocyclyl groups that have other groups such as alkyl or halo groups bonded to one of the ring members as compounds such as 2-methylbenzimidazolyl are substituted heterocyclyl groups. Examples of heterocyclyl groups include, but are not limited to: unsaturated 3 to 8 member rings containing 1 to 4 nitrogen atoms such as, but not limited to pyrrolyl, pyrrolinyl, imidazolyl, pyrazolyl, pyridyl, dihydropyridyl, pyrimidyl, pyrazinyl, pyridazinyl, triazolyl (e.g. 4H-1,2,4-triazolyl, 1H-1,2,3-triazolyl, 2H-1,2,3-triazolyl etc.), tetrazolyl, (e.g. 1H-tetrazolyl, 2H tetrazolyl, etc.); saturated 3 to 8 member rings containing 1 to 4 nitrogen atoms such as, but not limited to, pyrrolidinyl, imidazolidinyl, piperidinyl, piperazinyl; condensed unsaturated heterocyclic groups containing 1 to 4 nitrogen atoms such as, but not limited to, indolyl, isoindolyl, indolinyl, indolizinyl, benzimidazolyl, quinolyl, isoquinolyl, indazolyl, benzotriazolyl; unsaturated 3 to 8 membered rings containing 1 to 2 oxygen atoms and 1 to 3 nitrogen atoms such as, but not limited to, oxazolyl, isoxazolyl, oxadiazolyl (e.g. 1,2,4-oxadiazolyl, 1,3,4-oxadiazolyl, 1,2,5-oxadiazolyl, etc.); saturated 3 to 8 membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxazolyl, benzoxadiazolyl, benzoxazinyl (e.g. 2H-1,4-benzoxazinyl etc.); unsaturated 3 to 8 membered rings containing 1 to 3 sulfur atoms and 1 to 3 nitrogen atoms such as, but not limited to, thiazolyl, isothiazolyl, thiadiazolyl (e.g. 1,2,3-thiadiazolyl, 1,2,4-thiadiazolyl, 1,3,4-thiadiazolyl, 1,2,5-thiadiazolyl, etc.); saturated 3 to 8 member rings containing 1 to 2 sulfur atoms and 1 to 3 nitrogen atoms such as, but not limited to, thiazolodinyl; saturated and unsaturated 3 to 8 member rings containing 1 to 2 sulfur atoms such as, but not limited to, thienyl, dihydrodithiinyl, dihydrodithionyl, tetrahydrothiophene, tetrahydrothiopyran; unsaturated condensed heterocyclic rings containing 1 to 2 sulfur atoms and 1 to 3 nitrogen atoms such as, but not limited to, benzothiazolyl, benzothiadiazolyl, benzothiazinyl (e.g. 2H-1,4-benzothiazinyl, etc.), dihydrobenzothiazinyl (e.g. 2H-3,4-dihydrobenzothiazinyl, etc.), unsaturated 3 to 8 member rings containing oxygen atoms such as, but not limited to furyl; unsaturated condensed heterocyclic rings containing 1 to 2 oxygen atoms such as benzodioxolyl (e.g. 1,3-benzodioxoyl, etc.); unsaturated 3 to 8 member rings containing an oxygen atom and 1 to 2 sulfur atoms such as, but not limited to, dihydrooxathiinyl; saturated 3 to 8 member rings containing 1 to 2 oxygen atoms and 1 to 2 sulfur atoms such as 1,4-oxathiane; unsaturated condensed rings containing 1 to 2 sulfur atoms such as benzothienyl, benzodithiinyl; and unsaturated condensed heterocyclic rings containing an oxygen atom and 1 to 2 oxygen atoms such as benzoxathiinyl. Heterocyclyl group also include those described above in which one or more S atoms in the ring is double-bonded to one or two oxygen atoms (sulfoxides and sulfones). For example, heterocyclyl groups include tetrahydrothiophene, tetrahydrothiophene oxide, and tetrahydrothiophene 1,1-dioxide. Preferred heterocyclyl groups contain 5 or 6 ring members. More preferred heterocyclyl groups include morpholine, piperazine, piperidine, pyrrolidine, imidazole, pyrazole, 1,2,3-triazole, 1,2,4-triazole, tetrazole, thiomorpholine, thiomorpholine in which the S atom of the thiomorpholine is bonded to one or more O atoms, pyrrole, homopiperazine, oxazolidin-2-one, pyrrolidin-2-one, oxazole, quinuclidine, thiazole, isoxazole, furan, and tetrahydrofuran. The heterocyclyl group may be bonded to the parent molecule through any portion of the molecule, including the heteroatom portion.

The phrase “substituted heterocyclyl” refers to an unsubstituted heterocyclyl group as defined above in which one of the ring members is bonded to a non-hydrogen atom such as described above with respect to substituted alkyl groups and substituted aryl groups. Examples, include, but are not limited to, 2-methylbenzimidazolyl, 5-methylbenzimidazolyl, 5-chlorobenzthiazolyl, 1-methyl piperazinyl, and 2-chloropyridyl among others.

The phrase “unsubstituted heterocyclylalkyl” refers to unsubstituted alkyl groups as defined above in which a hydrogen or carbon bond of the unsubstituted alkyl group is replaced with a bond to a substituted or unsubstituted heterocyclyl group as defined above. For example, methyl (—CH₃) is an unsubstituted alkyl group. If a hydrogen atom of the methyl group is replaced by a bond to a heterocyclyl group, such as if the carbon of the methyl were bonded to carbon 2 of pyridine (one of the carbons bonded to the N of the pyridine) or carbons 3 or 4 of the pyridine, then the compound is an unsubstituted heterocyclylalkyl group. The heterocyclylalkyl group may be bonded to the parent molecule through any portion of the molecule, including the alkyl portion or the heterocyclyl portion.

The phrase “substituted heterocyclylalkyl” has the same meaning with respect to unsubstituted heterocyclylalkyl groups that substituted aralkyl groups had with respect to unsubstituted aralkyl groups. However, a substituted heterocyclylalkyl group also includes groups in which a non-hydrogen atom is bonded to a heteroatom in the heterocyclyl group of the heterocyclylalkyl group such as, but not limited to, a nitrogen atom in the piperidine ring of a piperidinylalkyl group.

### B. Compositions

1. SKF-96365

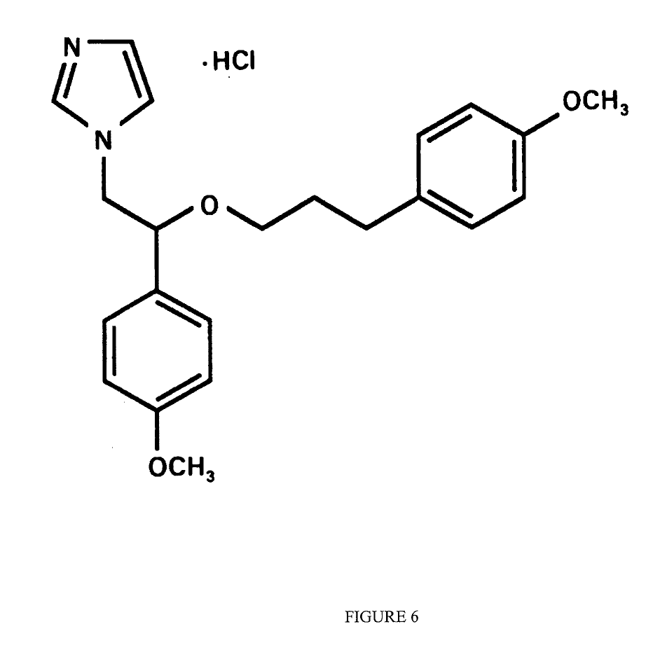

Useful compositions of the present disclosure comprise the small molecule SKF-96365 (1-[β-(3-(4-Methoxyphenyl)propoxy)-4-methoxyphenethyl]-1H-imidazole hydrochloride; CAS No. 130495-35-1) and/or derivatives thereof. The structure of this molecule is presented in FIG. 6. SKF-96365 is a voltage-independent calcium entry inhibitor (Merritt, J. E., et al., Biochem. J. 271, 515-522, (1990); Hotta, A., et al., J Smooth Muscle Res. 41(6):313-27, 2005), and has been unexpectedly discovered to prevent and reverse SMA and arrhythmia. These results suggest the unexpected potential of these compounds as novel anti-arrhythmic agents. SKF-96365 has been discovered to reverse SMA and arrhythmia once initiated, in addition to being effective to prevent them.

This present disclosure provides compounds of the general formula (I), or pharmaceutically acceptable salts thereof, esters thereof, tautomers and polymorphic variants of any of the foregoing.

Compounds of the general formula (I) have the following structure:

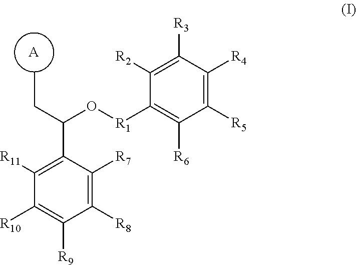

wherein,

R₁ is (CH₂)ₙ, where n is 0 to 10; in one embodiment, n is 1, 2, 3 or 4.

R₂ through R₁₁ are each independently selected from the group consisting of —H, —OH, halogens, alkyl, substituted alkyl, alkenyl, substituted alkenyl, alkynyl, substituted alkynyl, —NH₂, —NR₁₂R₁₃, —COR₁₄, —CON═N═N, —N═NR₁₅, —N═NOR₁₆, —N═NNR₁₇, —SR₁₈, —SOR₁₉, —SO₂R₂₀, —SO₃R₂₁, —OR₂₂ and —XR₂₃.

wherein

X is selected from the group consisting of: selenium (Se), tellurium (Te), polonium (Po) and technetium (Tc).

R₁₂, R₁₃, R₁₅, R₁₆ and R₁₇ and R₂₃ are each independently selected from the group consisting of: H, OH, alkyl, substituted alkyl, alkenyl, substituted alkenyl, alkynyl, and substituted alkynyl;

R₁₄, R₁₈, R₁₉, R₂₀ and R₂₁ are each independently selected from the group consisting of: H, OH, halogen, unsubstituted alkyl, substituted alkyl, alkenyl, substituted alkenyl, alkynyl, substituted alkynyl, and NR₁₂R₁₃;

R₂₂ is selected from the group consisting of: H, OH, halogen, unsubstituted alkyl, substituted alkyl, alkenyl, substituted alkenyl, alkynyl, substituted alkynyl COR₁₄, and NR₁₂R₁₃; and

A is selected from the group consisting of: a substituted heterocyclyl, an unsubstituted heterocyclyl, an unsubstituted heterocyclylalkyl, and a substituted heterocyclylalkyl.

In further specific embodiments, A comprises a three-member heterocyclyl, a four-member heterocyclyl, a five-member heterocyclyl, a six-member heterocyclyl, a seven-member heterocyclyl, or an eight-member heterocyclyl.

In further specific embodiments, A comprises a nitrogenous heterocyclyl, a sulfurous heterocyclyl, or a combination of the foregoing.

In further specific embodiments, A is selected from the group consisting of: imidazole, pyrrole, thiophene, thiazole, and pyrazole. In some embodiments of the compound, the imidazole, pyrrole, thiophene, thiazole, or pyrazole is substituted or unsubstituted. In still a further specific embodiment, A is an imadazole group of the structure:

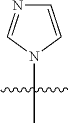

In some specific embodiments in which A comprises a substituted heterocyclyl, the heterocyclyl may be substituted with one or more groups selected from: —H, —OH, halogens, alkyl, substituted alkyl, alkenyl, substituted alkenyl, alkynyl, substituted alkynyl, —NH₂, —NR₂₄R₂₅, —COR₂₆, —CON═N═N, —N═NR₂₇, —N═NOR₂₈, —N═NNR₂₉, —SR₃₀, —SOR₃₁, —SO₂R₃₂, —SO₃R₃₃, —OR₃₄ and —ZR₃₅; Z is selected from the group consisting of selenium (Se), tellurium (Te), polonium (Po) and technetium (Tc); R₂₄, R₂₅, R₂₇, R₂₈ and R₂₉ and R₃₅ are each independently selected from the group consisting of: H, OH, alkyl, substituted alkyl, alkenyl, substituted alkenyl, alkynyl, and substituted alkynyl; R₂₆, R₃₀, R₃₁, R₃₂ and R₃₃ are each independently selected from the group consisting of: H, OH, halogen, unsubstituted alkyl, substituted alkyl, alkenyl, substituted alkenyl, alkynyl, substituted alkynyl, and NR₂₄R₂₅; R₃₄ is selected from the group consisting of: H, OH, halogen, unsubstituted alkyl, substituted alkyl, alkenyl, substituted alkenyl, alkynyl, substituted alkynyl COR₂₆, and NR₂₄R₂₅.

In further specific embodiments, R₂ through R₁₁ are each independently selected from the group consisting of:

—H

—[CH₂]ₘCH₃

—[CH₂]ₘCOOH

—O[CH₂]ₘCH₃

—S[CH₂]ₘCH₃

—SO₂[CH₂]ₘCH₃

—CO[CH₂]ₘCH₃

—OCO[CH₂]ₘCH₃

—OSiO[CH₂]ₘCH₃

—O(SO)O[CH₂]ₘCH₃

—SO[CH₂]ₘCH₃

—CONH[CH₂]ₘCH₃

—N═N[CH₂]ₘCH₃

—N═NO[CH₂]ₘCH₃

—N═NNH[CH₂]ₘCH₃

—(SO₄)[CH₂]ₘCH₃

—[CH₂]ₘCH₃

—Se[CH₂]ₘCH₃

—Tc[CH₂]ₘCH₃

Wherein m is from 0-10; in one embodiment, m is 0, 1, 2, 3 or 4.

In specific embodiments, one of R₂ through R₆ is selected from the group consisting of H, OH, halogens, alkyl, substituted alkyl, alkenyl, substituted alkenyl, alkynyl, substituted alkynyl, OR₂₂, NH₂, NR₁₂R₁₃ and SR₁₄ and the remaining are H, one of R₇ through R₁₁ is selected from the group consisting of H, OH, halogens, alkyl, substituted alkyl, alkenyl, substituted alkenyl, alkynyl, substituted alkynyl, OR₂₂, NH₂, NR₁₂R₁₃ and SR₁₄ and the remaining are H, or a combination of the foregoing.

In an additional specific embodiment, one of R₂ through R₆ is OCH₃, and the remaining are H, and one of R₇ through R₁₁ is OCH₃, and the remaining are H. In a further specific embodiment, R₄ and R₉ are OCH₃, and the remainder of R₂ through R₃, R₅ through R₈ and R₉ through R₁₁ are H.

In another specific embodiment, R₁ is (CH₂)₃.

In yet another specific embodiment, the compound of the general formula (I) is SKF-96365 and has the following structure:

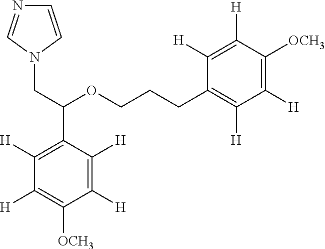

In one embodiment of the composition, the pharmaceutically acceptable salt of a compound, such as SKF-96365, is used. In a particular embodiment, the pharmaceutically acceptable salt is the hydrochloride. The phrase “pharmaceutically acceptable salt(s)”, as used herein, means those salts of compounds that are safe and effective for use in subjects and that possess the desired biological activity. Pharmaceutically acceptable salts include salts of acidic or basic groups. A pharmaceutically acceptable salt includes a salt with an inorganic base, organic base, inorganic acid, organic acid, or basic or acidic amino acid. As salts of inorganic bases, the invention includes, for example, alkali metals such as sodium or potassium; alkaline earth metals such as calcium and magnesium or aluminum; poor metals, such as bismuth; and ammonia. As salts of organic bases, the invention includes, for example, trimethylamine, triethylamine, pyridine, picoline, ethanolamine, diethanolamine, and triethanolamine. As salts of inorganic acids, the instant invention includes, for example, hydrochloric acid, hydroboric acid, nitric acid, hydroiodic acid, sulfuric acid, hydrobromic acid, and phosphoric acid. As salts of organic acids, the instant invention includes, for example, formic acid, acetic acid, trifluoroacetic acid, fumaric acid, oxalic acid, tartaric acid, maleic acid, citric acid, succinic acid, malic acid, methanesulfonic acid, benzenesulfonic acid, glucuronic acid, acistric acid, besylic acid, tosylic acid, xinafoic acid, isonicotinic acid, lactic acid, salicylic acid, pantothenic acid, bitartic acid, ascorbic acid, gentisinic acid, gluconic acid, glucaronic acid, saccharic acid, benzoic acid, glutamic acid, methanesulfonic acid, ethanesulfonic acid, benzensulfonic acid, pamoic acid, and p-toluenesulfonic acid. As salts of basic amino acids, the instant invention includes, for example, arginine, tromethamine, lysine and ornithine. Acidic amino acids include, for example, aspartic acid and glutamic acid.

The selection of a pharmaceutically acceptable salt depends on numerous factors understood by those skilled in the art, including but not limited to hygroscopicity, solubility, stability, and absorptiveness. Factors to be considered in choosing a pharmaceutically acceptable salt are further described in Remington: The Science and Practice of Pharmacy (20th Ed., Lippincott, Williams & Wilkins, Daniel Limmer, editor), on pages 706-713.

In some embodiments, pharmaceutically acceptable derivatives of SKF-96365 may be used. Such derivatives can be any derivatives understood by those skilled in the art to posses the ability to inhibit arrhythmia or SMA in one or more of the model systems described herein or known in the art and which derivatives fall within the definition of compound (I) described herein. Those skilled in the art are capable of producing derivatives through various methods, including but not limited to homolog series, molecular fragmentation, the addition of functional groups, isosteric replacement, stereoisomeric rearrangement, and ionic substitution.

2. Pharmaceutical Compositions

In one embodiment, such compounds are in the form of compositions, such as but not limited to, pharmaceutical compositions and medicaments. The compositions disclosed may comprise one or more of such compounds, in combination with a pharmaceutically acceptable carrier. Examples of such carriers and methods of formulation may be found in Remington: The Science and Practice of Pharmacy (20th Ed., Lippincott, Williams & Wilkins, Daniel Limmer, editor). To form a pharmaceutically acceptable composition suitable for administration, such compositions will contain a therapeutically effective amount of a compound(s).

The pharmaceutical compositions of the disclosure may be used in the treatment and prevention methods of the present disclosure. Such compositions are administered to a subject in amounts sufficient to deliver a therapeutically effective amount of the compound(s) so as to be effective in the methods disclosed herein. The therapeutically effective amount may vary according to a variety of factors such as, but not limited to, the subject's condition, weight, sex and age. Other factors include the mode and site of administration. The pharmaceutical compositions may be provided to the subject in any method known in the art. Exemplary routes of administration include, but are not limited to, subcutaneous, intravenous, topical, epicutaneous, oral, intraosseous, intramuscular, intranasal and pulmonary. In some embodiments, the therapeutically effective amount or effective inhibitory amount will be sufficient to achieve an extracellular concentration of the compound at or below about 10 μM. In some embodiments, the therapeutically effective amount or effective inhibitory amount will be sufficient to achieve an extracellular concentration of 10 μM, 25 μM, 50 μM, 75 μM, 100 μM, 150 μM, 200 μM, 300 μM, about any of the forgoing concentrations, or at least any of the foregoing concentrations. In one embodiment the compound is SKF-96365 or a pharmaceutically acceptable salt of SKF-96365

The compositions of the present disclosure may be administered only one time to the subject or more than one time to the subject. Furthermore, when the compositions are administered to the subject more than once, a variety of regimens may be used, such as, but not limited to, one per day, once per week, once per month or once per year. The compositions may also be administered to the subject more than one time per day. The therapeutically effective amount of the molecules and appropriate dosing regimens may be identified by routine testing in order to obtain optimal activity, while minimizing any potential side effects. In addition, co-administration or sequential administration of other agents may be desirable.

The compositions of the present disclosure may be administered systemically, such as by intravenous administration, or locally such as by subcutaneous injection or by application of a paste or cream.

The compositions of the present disclosure may further comprise agents which improve the solubility, half-life, absorption, etc. of the compound(s). Furthermore, the compositions of the present disclosure may further comprise agents that attenuate undesirable side effects and/or or decrease the toxicity of the compounds(s). Examples of such agents are described in a variety of texts, such a, but not limited to, Remington: The Science and Practice of Pharmacy (20th Ed., Lippincott, Williams & Wilkins, Daniel Limmer, editor).

The compositions of the present disclosure can be administered in a wide variety of dosage forms for administration. For example, the compositions can be administered in forms, such as, but not limited to, tablets, capsules, sachets, lozenges, troches, pills, powders, granules, tinctures, solutions, suspensions, elixirs, syrups, ointments, creams, pastes, emulsions, or solutions for intravenous administration or injection. Other dosage forms include administration transdermally, via patch mechanism or ointment. Further dosage forms include formulations suitable for delivery by nebulizers or metered dose inhalers. Any of the foregoing may be modified to provide for timed release and/or sustained release formulations.

In the present disclosure, the pharmaceutical compositions may further comprise a pharmaceutically acceptable carrier. Such carriers include, but are not limited to, vehicles, adjuvants, surfactants, suspending agents, emulsifying agents, inert fillers, diluents, excipients, wetting agents, binders, lubricants, buffering agents, disintegrating agents and carriers, as well as accessory agents, such as, but not limited to, coloring agents and flavoring agents (collectively referred to herein as a carrier). Typically, the pharmaceutically acceptable carrier is chemically inert to the active compounds and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

For instance, for oral administration in solid form, such as but not limited to, tablets, capsules, sachets, lozenges, troches, pills, powders, or granules, the compound(s) may be combined with an oral, non-toxic pharmaceutically acceptable inert carrier, such as, but not limited to, inert fillers, suitable binders, lubricants, disintegrating agents and accessory agents. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthum gum and the like. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid as well as the other carriers described herein. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

For oral liquid forms, such as but not limited to, tinctures, solutions, suspensions, elixirs, syrups, the molecules of the present disclosure can be dissolved in diluents, such as water, saline, or alcohols. Furthermore, the oral liquid forms may comprise suitably flavored suspending or dispersing agents such as the synthetic and natural gums, for example, tragacanth, acacia, methylcellulose and the like. Moreover, when desired or necessary, suitable coloring agents or other accessory agents can also be incorporated into the mixture. Other dispersing agents that may be employed include glycerin and the like.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound(s) may be administered in a physiologically acceptable diluent, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as, but not limited to, a soap, an oil or a detergent, suspending agent, such as, but not limited to, pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include polyethylene sorbitan fatty acid esters, such as sorbitan monooleate and the high molecular weight adducts of ethylene oxide with a hydrophobic base, formed by the condensation of propylene oxide with propylene glycol, oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkylbeta-aminopropionates, and 2-alkylimidazoline quaternary ammonium salts, and (e) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17.

Topical dosage forms, such as, but not limited to, ointments, creams, pastes, emulsions, containing the molecule of the present disclosure, can be admixed with a variety of carrier materials well known in the art, such as, e.g., alcohols, aloe vera gel, allantoin, glycerine, vitamin A and E oils, mineral oil, PPG2 myristyl propionate, and the like, to form alcoholic solutions, topical cleansers, cleansing creams, skin gels, skin lotions, and shampoos in cream or gel formulations. Inclusion of a skin exfoliant or dermal abrasive preparation may also be used. Such topical preparations may be applied to a patch, bandage or dressing for transdermal delivery or may be applied to a bandage or dressing for delivery directly to the site of a wound or cutaneous injury.

The compound(s) of the present disclosure can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. Such liposomes may also contain monoclonal antibodies to direct delivery of the liposome to a particular cell type or group of cell types.

The compound(s) of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyl-eneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

### C. Methods of Treatment and Prevention of Arrhythmia

Methods of treatment and prevention of arrhythmia are now possible based on the unexpectedly discovered anti-arrhythmic properties of SKF-96365 and derivatives thereof.

Embodiments of the method include a method of preventing arrhythmia in a subject, the method comprising administering to the subject a therapeutically effective amount of a compound of the general formula (I), a derivative thereof, a tautomer of any of the foregoing, or a polymeric variant of any of the foregoing or a pharmaceutically acceptable salt of any of the foregoing. In one embodiment, the compound is SKF-96365, a derivative thereof, a tautomer of any of the foregoing, or a polymeric variant of any of the foregoing or a pharmaceutically acceptable salt of any of the foregoing. In further embodiments of the method, the arrhythmia comprises an automatic ectopic event. In further embodiments of the method, the arrhythmia comprises a reentrant ectopic event, a triggered ectopic event, an automatic ectopic event, a sporadic automatic ectopic event, a tachycardic automatic ectopic event or a fibrillation-like chaotic activity. In further embodiments of the method, the method comprises identifying a subject in need of prevention of arrhythmia.

Further embodiments of the method include a method of treating arrhythmia in a subject, the method comprising administering to the subject a therapeutically effective amount of a compound of the general formula (I), a derivative thereof, a tautomer of any of the foregoing, or a polymeric variant of any of the foregoing or a pharmaceutically acceptable salt of any of the foregoing. In one embodiment, the compound is SKF-96365, a derivative thereof, a tautomer of any of the foregoing, or a polymeric variant of any of the foregoing or a pharmaceutically acceptable salt of any of the foregoing. In further embodiments of the method, the arrhythmia comprises an automatic ectopic event. In further embodiments of the method, the arrhythmia comprises a triggered ectopic event. In further embodiments of the method, the arrhythmia comprises a triggered ectopic event, an automatic ectopic event, a sporadic automatic ectopic event, a tachycardic automatic ectopic event or a fibrillation-like chaotic activity. In further embodiments of the method, the method comprises identifying a subject in need of treatment of arrhythmia.

In embodiments of the method comprising identifying a subject in need of treatment or prevention of arrhythmia, the identification of the subject may occur prior to administration of inhibitor. However, under conditions in which the administration of inhibitor has diagnostic value, the identification of the subject may occur after administration. Compounds may be administered in a single dose, at multiple doses administered over time or in any other manner known in the art. Compounds may be administered alone or in combination with other compounds. When a combination is administered, the individual compounds may be administered simultaneously, each at a given dosage, or they may be interspersed at varying, intermittent, or alternating dosages and times. Administration may occur at any time relative to an onset of arrhythmia. Administration may occur in a patient who has never experienced arrhythmia, presumably for preventive purposes. Administration may occur at a time after a patient has experienced a discrete occurrence of arrhythmia, generally (but not necessarily) to prevent a recurrence. Administration may occur during an arrhythmia event, to reverse the arrhythmia and restore normal rhythmic function to the heart.

Some embodiments of the method comprise administering a therapeutically effective amount of the compound to the subject.

### D. Methods OF Preventing And Reversing Arrhythmia in Cardiac Muscle

The disclosure provides methods for both reversing and preventing arrhythmia in a cardiac muscle. The cardiac muscle may be part of a heart; if the cardiac muscle is part of a heart, it may be intact in the living animal or isolated from the living animal (for example, a perfused heart). The cardiac muscle may alternatively be isolated, although the method can be performed for any cardiac muscle.

Methods for reversing arrhythmia in a cardiac muscle are provided. One embodiment of the method comprises contacting the muscle with a therapeutically effective amount of a compound of the general formula (I), a derivative thereof, a tautomer of any of the foregoing, or a polymeric variant of any of the foregoing or a pharmaceutically acceptable salt of any of the foregoing. In one embodiment, the compound is SKF-96365, a derivative thereof, a tautomer of any of the foregoing, or a polymeric variant of any of the foregoing or a pharmaceutically acceptable salt of any of the foregoing. In one embodiment of the method, the arrhythmia comprises a triggered ectopic event, automatic ectopic events, sporadic automatic ectopic events, a tachycardic automatic ectopic events or a fibrillation-like chaotic activity. In additional embodiments of the method, the cardiac muscle is that of a living subject. In additional embodiments of the method the cardiac muscle is that of an intact heart. In additional embodiments of the method the muscle is an isolated cardiac muscle.

Methods of preventing arrhythmia in a cardiac muscle are also provided. In certain embodiments of the method, the method comprises contacting the muscle with a therapeutically effective amount of a compound of the general formula (I), a derivative thereof, a tautomer of any of the foregoing, or a polymeric variant of any of the foregoing or a pharmaceutically acceptable salt of any of the foregoing. In one embodiment, the compound is SKF-96365, a derivative thereof, a tautomer of any of the foregoing, or a polymeric variant of any of the foregoing or a pharmaceutically acceptable salt of any of the foregoing. In one embodiment of the method, the arrhythmia comprises a triggered ectopic event, automatic ectopic events, sporadic automatic ectopic events, a tachycardic automatic ectopic events or a fibrillation-like chaotic activity. In additional embodiments of the method the muscle is that of a living subject. In additional embodiments of the method the muscle is that of an intact heart. In additional embodiments of the method the muscle is an isolated cardiac muscle.

Some embodiments of the method comprise contacting the muscle with a therapeutically effective amount of the compound.

### E. Methods of Inhibiting Spontaneous Mechanical Activity

Methods are provided for inhibiting SMA in a myocyte. The myocyte can be found in any setting, including but not limited to a living animal, tissue culture, other in vitro settings, or as part of an isolated heart or cardiac muscle.

Certain embodiments of the method comprise contacting the myocyte with an effective inhibitory concentration of a compound of the general formula (I), a derivative thereof, a tautomer of any of the foregoing, or a polymeric variant of any of the foregoing or a pharmaceutically acceptable salt of any of the foregoing. In one embodiment, the compound is SKF-96365, a derivative thereof, a tautomer of any of the foregoing, or a polymeric variant of any of the foregoing or a pharmaceutically acceptable salt of any of the foregoing. SMA may give rise to arrhythmias as described herein and known in the art. In some embodiments of the method, the arrhythmia comprises a triggered ectopic event, automatic ectopic events, sporadic automatic ectopic events, a tachycardic automatic ectopic events or a fibrillation-like chaotic activity. In certain embodiments, contacting occurs during an occurrence of SMA; in this case, the inhibition of SMA takes the form of reversing SMA in the myocyte. In certain embodiments, contacting occurs prior to or in the absence of an occurrence of SMA; in this case, the inhibition of SMA takes the form of preventing SMA in the myocyte. In additional embodiments of the method the myocyte is a component of an intact heart, either in a living subject or a perfused heart. In additional embodiments of the method the myocyte is part of an isolated cardiac muscle.

Some embodiments of the method comprise contacting the myocyte with a therapeutically effective amount of the compound.

### F. Kits

The present disclosure provides kits for carrying out any method of the present disclosure, which can contain any of the compounds and/or compositions disclosed herein or otherwise useful for practicing a method of the disclosure.

For example, the disclosure provides a kit for the treatment or prevention of arrhythmia in a subject, the kit comprising a dosage form of a composition containing a compounds of the formula (I), a derivative thereof, a tautomer of any of the foregoing, or a polymeric variant of any of the foregoing or a pharmaceutically acceptable salt of any of the foregoing. In one embodiment the compound is SKF-96365, a derivative thereof, a tautomer of any of the foregoing, or a polymeric variant of any of the foregoing or a pharmaceutically acceptable salt of any of the foregoing. In another embodiment, the composition is a pharmaceutical composition. In a specific embodiment, the dosage may contain a therapeutically effective amount of any compound or composition of this disclosure. The pharmaceutical may include additional pharmaceutically useful components as described in the preceding sections.

The kit may further comprise instructions for administering the dosage form.

### G. Model Systems of Arrhythmia

1. Development of a Model System for Arrhythmia

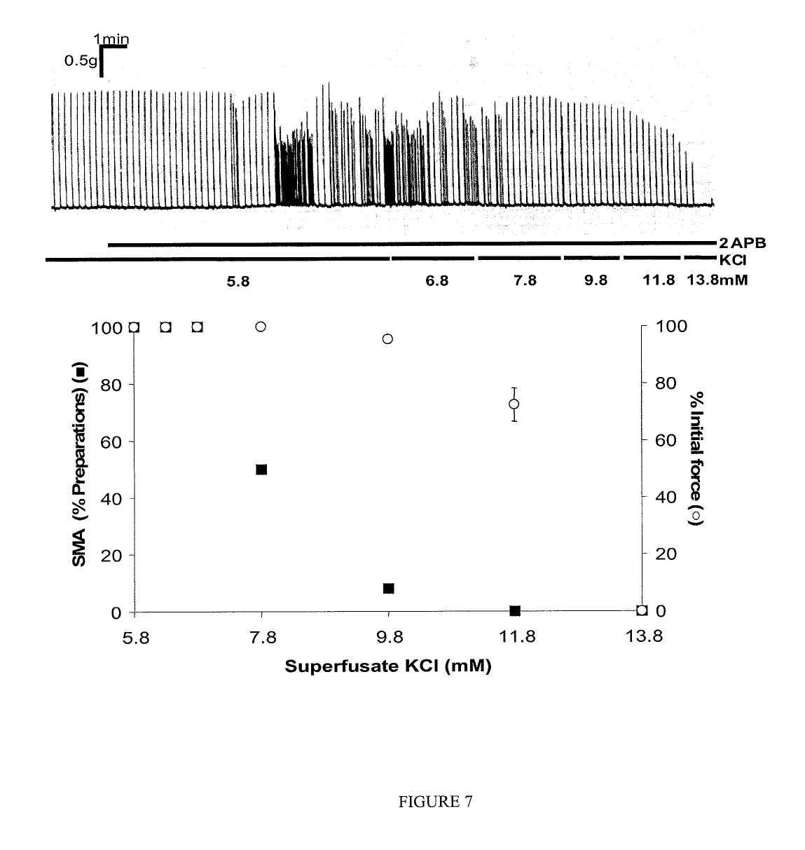

Without wishing to be bound by any given theoretical model, the present disclosure hypothesized that unregulated myocyte calcium release or leak may be responsible for the occurrence of SMA and arrhythmia. Since sarcoplasmic reticulum (SR) calcium leak may underlie these contractile irregularities, experiments were conducted to determine whether 2-aminoethoxydiphenyl borate (2-APB), a calcium leak-inducer, affects mechanical function in isolated, superfused rat left atria (in the context of this example, the term “left atria” encompasses the use of the left atrial appendage). Exposing left atria paced at 3 Hz to >10 μM 2-APB produced sporadic mechanical events that occurred in the absence of pacing stimulus. Prolonging atrial diastole in the presence of 2-APB produced SMA. SMA depends on atrial sodium and chloride gradients as decreasing superfusate concentration of either ion suppressed SMA. Increasing superfusate potassium to produce an B_{K} of about −74 mV reversed SMA (FIG. 7), revealing possible membrane potential sensitivity. Mechanical function decreased with time in left atria treated with 2-APB and low sodium or the anion transport inhibitor 4,4′-diisothiocyanatostilbene-2,2′-disulfonic acid (DIDS) compared with atria exposed to low sodium or DIDS alone, suggesting 2-APB may decrease left atrial SR activator calcium. Thus, 2-APB produces instability in regular left atrial mechanical activity that may require forward-mode sodium-calcium exchange (NCX) and chloride channel activities. These data identify a novel model for studying atrial contractile abnormalities, such as but not limited to, arrhythmia and SMA.

Calcium leakage can activate depolarizing currents that produce ectopic mechanical events in isolated myocytes. However few models are available to investigate whether calcium leakage underlies ectopic mechanical events in intact heart muscle preparations. Experimental results described below demonstrate that 2-APB produces instability in the regular mechanical activity of isolated rat left atria (i.e., spontaneous mechanical events (SMEs) and SMA) and depresses the maximal mechanical function of these isolated preparations, a measure of SR activator calcium. These results identify a new pharmacological model with potential in studying mechanical abnormalities in isolated left atria under controlled conditions.

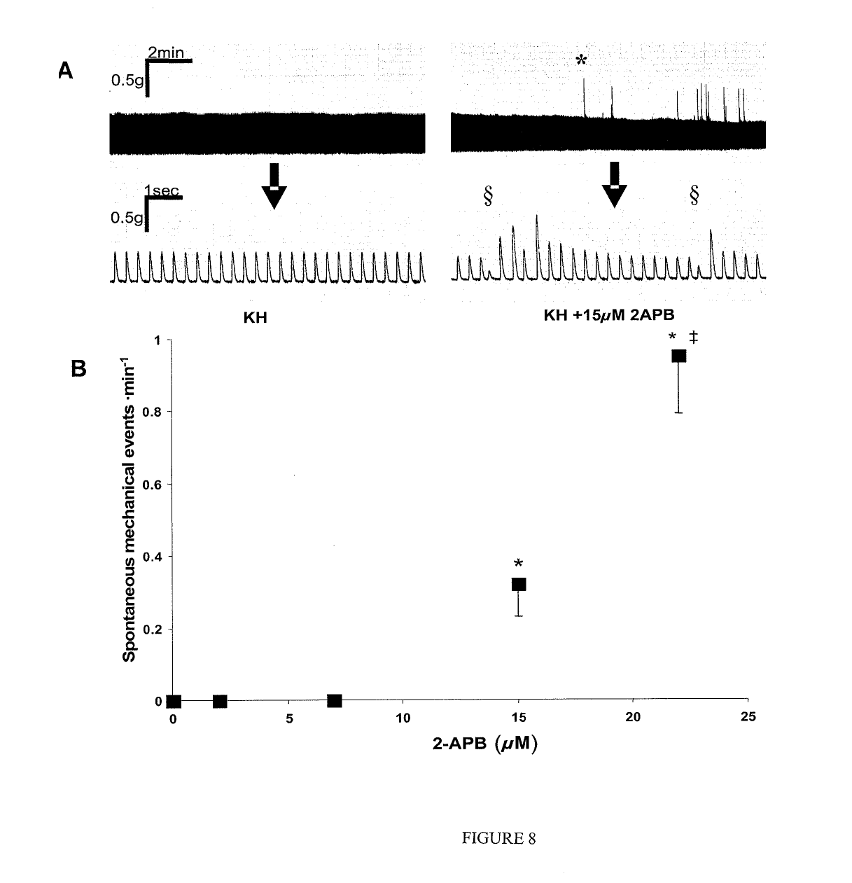

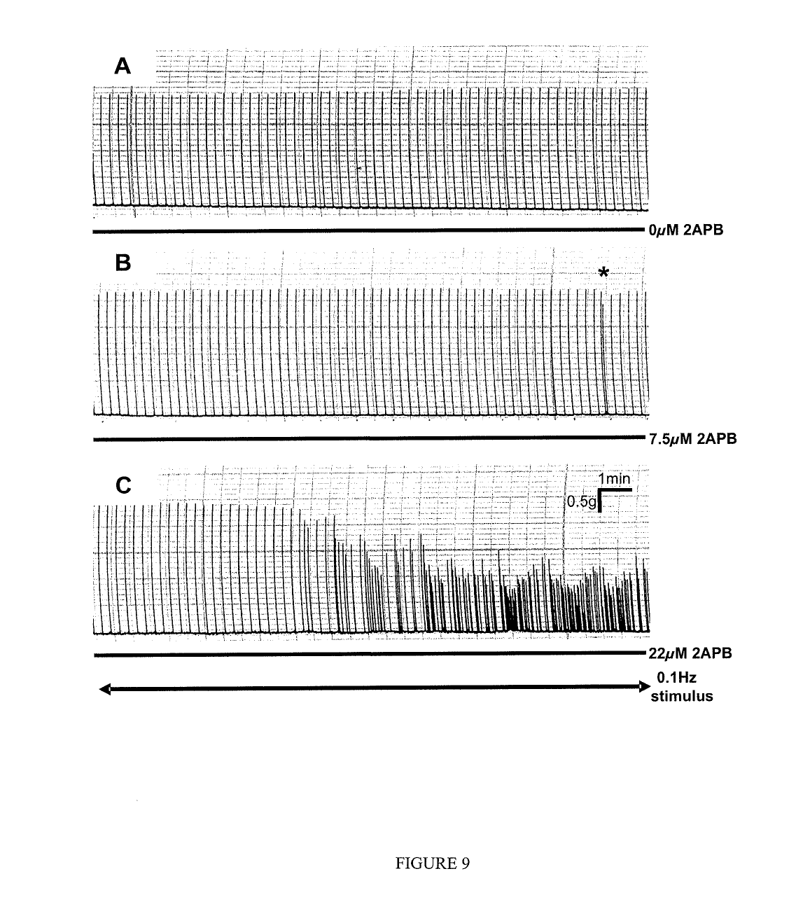

Instability of regular mechanical function was observed to occur only in left atria exposed to 2-APB (FIG. 8) and became more pronounced as diastolic interval was lengthened. In the extreme, at pacing rates of 0.1 Hz and 2-APB concentrations of 15 μM numerous atrial mechanical events occurred in the absence of a pacing stimulus, SMA (FIG. 9). SMA appears to result from a specific interaction between 2-APB and left atria rather than from general atrial disruption, since diastolic tension did not increase in 2-APB-treated preparations (FIG. 9), and removing 2-APB from the superfusate reversed its effects on atrial mechanical function (data not shown).

Without wishing to be limited by any hypothetical model, one interpretation of these results is that SMA occurs as a result of depolarizations that arise in left atria exposed to moderate concentrations of 2-APB. Experimental evidence shows that increased forward-mode NCX activity can cause fluctuations in myocyte membrane potential that trigger aberrant depolarizations. It has been suggested that calcium-activated chloride channels also contribute to these fluctuations, which elicit spontaneous depolarizations and ectopic mechanical events. Since NCX and calcium-activated chloride channels have different kinetic properties and appear to access different pools of myocyte calcium, some have hypothesized that both forward-mode NCX and calcium-activated chloride channels contribute to producing ectopic mechanical events. Alternatively, calcium leakage from internal calcium stores may activate a type of sodium channel distinct from the transient sodium current which provokes ectopic depolarizations.

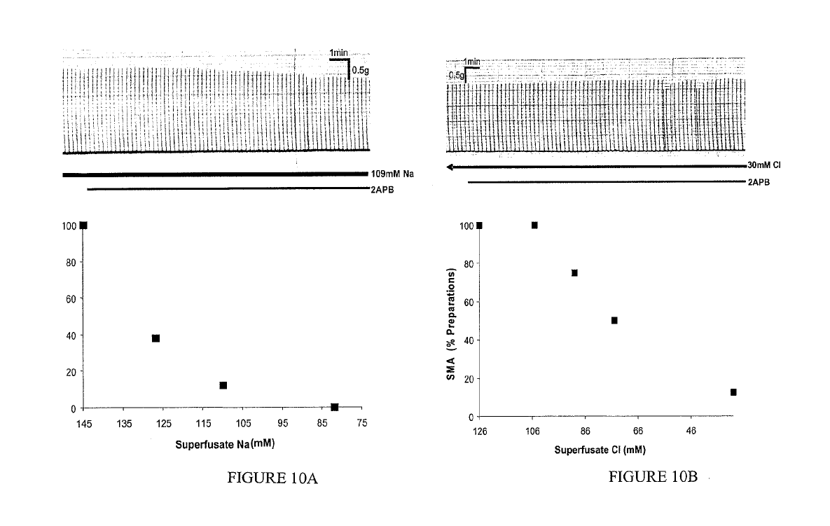

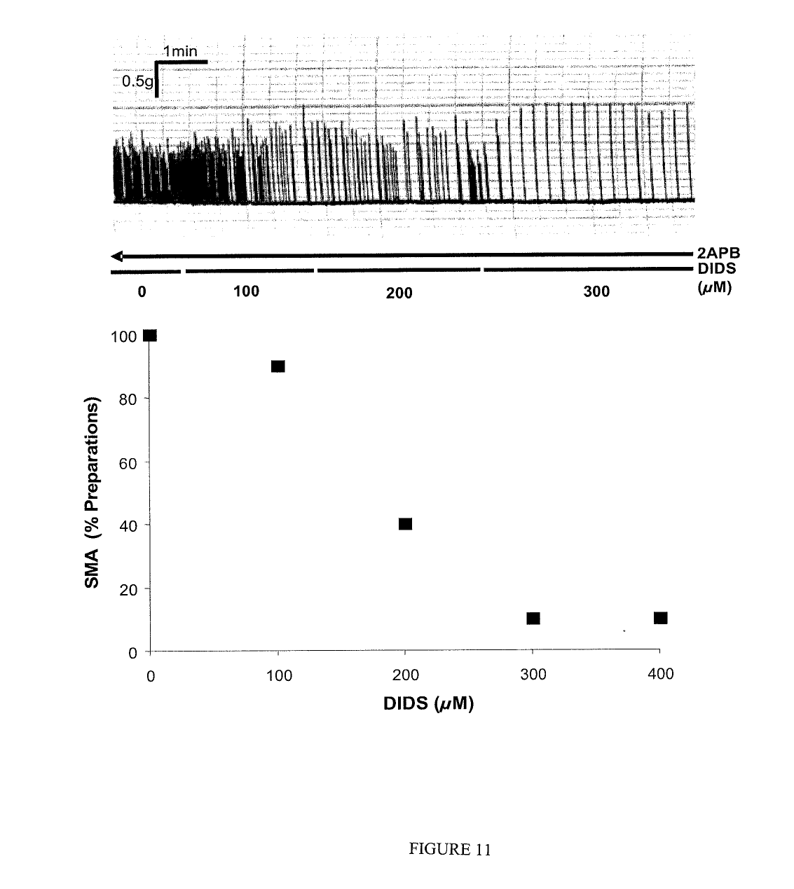

Six sets of results address this question. Decreasing either superfusate sodium or chloride prevented and reversed SMA (FIGS. 10A and B). DIDS also reversed SMA at concentrations shown previously to block anion transport (FIG. 11). These results suggest that both sodium and chloride gradients and anion transporters may be important in initiating and maintaining SMA. The fact that increasing superfusate potassium also reversed SMA (FIG. 7) suggests a membrane potential-sensitive step mediates, in part, SMA. These increases in superfusate potassium produced a calculated E_{K} of ˜−74 mV. Such a change, under conditions of normal superfusate sodium and calcium, may suppress forward-mode NCX activity activated by an increase in intracellular calcium. Taken together, the ion substitution and transport inhibitor studies suggest a role for both forward-mode NCX activity and chloride channel activity in initiating and maintaining left atrial SMA. Alternatively, calcium leakage from internal calcium stores may activate a type of sodium channel which provokes ectopic depolarizations and exhibits sensitivity to external sodium and trans-membrane potential distinct from the sodium channel which produces the transient sodium current.

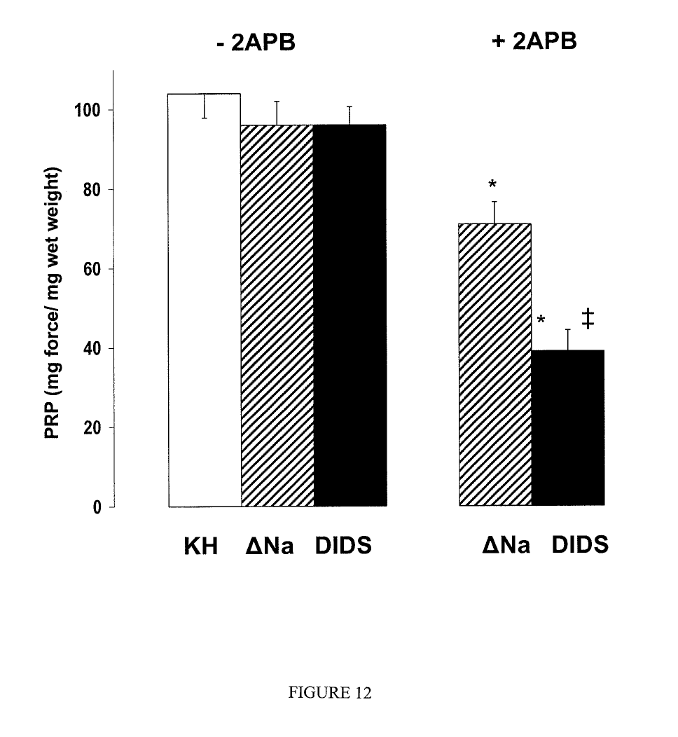

Lowering superfusate sodium or DIDS treatment acutely reversed SMA despite the continued presence of 2-APB (FIGS. 10A and 11). Prolonged superfusion of 2-APB-treated left atria in the presence of low sodium or DIDS produced a decline in the maximum force of atrial contraction when compared with preparations treated with low sodium or DIDS alone (FIG. 12). Others have reported that the first potentiated beat following prolonged rest, the maximum force of contraction, reflects the SR content of activator calcium. The present results suggest that 2-APB decreases the activator calcium available to generate mechanical force in isolated left atria. Importantly, atrial mechanical function was better preserved in the presence of 2-APB when low sodium was used to reverse SMA compared with when DIDS was used (FIG. 12; Right bars). The accelerated loss of maximum mechanical force in (2-APB+DIDS)-treated atria suggests that the activator calcium pool may decrease more in atria where forward-mode NCX activity should be unfettered. In contrast, lowering superfusate sodium should suppress forward-mode NCX activity, so this procedure to reverse SMA might slow the loss of atrial calcium via NCX and better preserve atrial mechanical performance. Under conditions of low superfusate sodium, sarcolemmal calcium ATPase may be responsible for calcium efflux from atrial myocytes.

Again without wishing to be limited by any hypothetical model, alternative explanations present themselves for the decreased maximum mechanical performance occurring in the presence of 2-APB. One is that 2-APB alters atrial myofilament calcium sensitivity either directly or by altering atrial metabolism to produce acidosis. The fact that atrial contraction and relaxation times were similar in left atria undergoing SMA or regular mechanical activity (Table 1) is inconsistent with this possibility as atrial relaxation times might change if altered myofilament calcium sensitivity occurred in 2-APB-treated preparations. Nonetheless, direct measure of myofilament calcium sensitivity is required to establish whether sensitivity is constant under the conditions described here. A second alternative explanation is that 2-APB alters atrial myocyte SR calcium release and accumulation. However, the similar contraction and relaxation times measured in the presence and absence of 2-APB again suggests that SR calcium release and accumulation are not greatly affected here. Third, 2-APB may alter the activity of atrial ion channels, including the slow calcium and sodium channels.

TABLE 1
Contraction and relaxation times of
untreated and 2-APB-treated rat left atria

| | | TPT | T0.9R | T0.5R |
|---|---|---|---|---|
| Group | Measures | (msec) | (msec) | (msec) |
| Control | 13 | 63 ± 2.0 | 75 ± 2.8 | 35 ± 1.3 |
| 2-APB | 11 | 59 ± 1.6 | 74 ± 0.8 | 35 ± 0.8 |

Control left atrial appendages were paced at 3 Hz while 2-APB values were obtained during SMA.
The “Measures” column indicates the number of independent measurements in each group;
the “TPT” column indicates the time to peak tension;
the “T0.9R” column indicates the time to 90% relaxation;
the “T0.5R” column indicates the time to 50% relaxation.
Data are mean ± SEM.

Some properties specific to atrial compared with ventricular myocytes may afford unique triggers for instability in regular left atrial mechanical activity. It has been reported that enhanced IP3 receptor (IP3R) calcium signaling leads to membrane depolarization and SR calcium release in isolated atrial myocytes and suggested that these abnormalities occur via NCX and/or the calcium-activated chloride channel activity present in atrial myocytes. Earlier reports also show high levels of type 2 IP3R occur in atrial compared with ventricular myocytes and that IP3R signaling can contribute to instability in atrial myocyte mechanical function. Finally, moderate concentrations of 2-APB alter the coupling process between IP3R and SOCs in non-excitable cells, causing leakage from store-operated calcium depots. The latter event, if it occurs in isolated left atria, could contribute to results reported here. Indeed, it is believed that 2-APB provokes voltage-independent SOC calcium entry over the concentration range which elicits SMA in rat left atria (Peinelt C. et al. “2-Aminoethoxydiphenyl borate directly facilitates and indirectly inhibits STIM1-dependent gating of CRAC channels.” Journal of Physiology 586: 3061-3073 (2008), which is hereby incorporated by reference for such teaching).

The conclusions that can be drawn from the experiments described below are that moderate concentrations of 2-APB produce left atrial SMA, instability in the regular mechanical activity of these isolated muscles, which appears to require forward-mode NCX and chloride channel activities. These data identify a pharmacological approach that produces atrial ectopy that may contribute to arrhythmias.

a. Material and Methods

i. Preparation of Isolated Rat Left Atria and Measurement of Atrial Mechanical Function

Left atria were isolated from male Sprague-Dawley rats (325 to 400 g) under IP pentobarbital anesthesia (0.1 g/kg). Isolated left atria were superfused at 30° C. in Krebs-Henseleit (KH) buffer of the following mM composition: NaCl, 118; NaHCO₃, 27; KCl, 4.8; MgSO₄, 1.2; KH₂PO₄, 1.0; CaCl₂, 1.8; and glucose, 11.1. Nearly isometric left atrial forces of contraction were measured as described.

ii. Four Protocols Analyzing the Effect of 2-APB on Atrial Mechanical Function

Isolated superfused left atria were paced at 3 Hz. 2-APB was added to atrial superfusates from 0.015 or 0.15M DMSO stock solutions to achieve concentrations of 0, 2, 7.5, 15, or 22 μM (n=5 to 10 atria per concentration). Changes in atrial mechanical force were recorded during a subsequent 15 min superfusion. DMSO alone did not affect atrial mechanical function.

Two groups of 5 to 6, 3 Hz paced atria were superfused in KH and exposed to 0 or 15 μM 2-APB for 10 min. The pacing stimulus was interrupted for 5 min and the number of spontaneous mechanical events (SMEs), defined as increases in mechanical force occurring in the absence of pacing, were measured as was the postrest potentiation response (PRP).

To determine whether diastolic interval affected the interaction between 2-APB and left atria, atrial pacing was decreased to 0.1 Hz, and 0, 7.5, or 22 μM 2-APB was added to the superfusate (n=6-8 per group). Superfusions continued for 15 min, and changes in atrial mechanical function were observed. Under these conditions, numerous SMEs occurred, and this phenomenon was designated SMA.

To establish whether SMA was reversible, atria (n=7) were paced at 0.1 Hz, exposed to 15 μM 2-APB for 10 min, and washed with KH. The effect of removing 2-APB on atrial mechanical function was recorded.

iii. Four Protocols Analyzing the Effect of Altering Superfusate Ion Composition and an Anion Transport Inhibitor on SMA

A prolonged diastolic interval was used here to highlight SMA and to favor the consequences of calcium leak on left atrial mechanical function.

To test whether lowering superfusate sodium affected SMA, 3 groups of left atria (n=6-8 per group) were paced at 0.1 Hz and washed with KH containing 127, 109, or 82 mM sodium. Choline chloride was used to maintain constant ionic strength and constant total chloride of 126 mM.

To test whether lowering superfusate chloride affected SMA, 4 groups of left atria (n=6-8 per group) were paced at 0.1 Hz and washed with KH containing 106, 89, 72, or 30 mM total chloride. Sodium glucuronate was used to maintain constant ionic strength and constant total sodium of 145 mM. Fifteen minutes after washing with modified KH, 15 μM 2-APB was added to the superfusate, and the occurrence of SMA was observed during a subsequent 15 min superfusion.

To test whether an anion transport inhibitor affected SMA, atria (n=7) were paced at 0.1 Hz in KH and exposed to 15 μM 2-APB. Following the appearance of SMA, they were titrated with 100 to 400 μM DIDS for 3 to 5 min at each concentration. Whether SMA occurred at the end of each titration period was recorded.

To determine whether decreasing B_{K} affects SMA, left atria were treated with 0 or 15 μM 2-APB (n=7 per group). Ten minutes later, aliquots of 4M KCl were added to the superfusate to increase potassium incrementally to 14 mM. Basal force of contraction was measured in both groups, and the occurrence of SMA was recorded in 2-APB-treated atria.

iv. Five Protocols Evaluating Whether 2-APB Affects SR Activator Calcium in Intact Atria

The initial potentiated beat following prolonged rest (i.e., the PRP maximum force of contraction) reflects activator calcium available for SR release. The PRP response was used to obtain an indirect measure of left atrial SR calcium under our conditions. A 1 min rest was chosen since forces of contraction occurred with ˜30 sec of rest (data not shown).

Left atria (n=6) were superfused in KH, paced at 0.1 Hz, and subjected to PRP. Superfusion proceeded for another 35 min, and a second PRP was performed. These 2 forces were compared to assure the mechanical stability of our preparations.

Two groups of left atria determined how 2-APB affects the maximum atrial force of contraction under conditions of decreased sodium.

One group (n=8) was superfused in KH, paced at 0.1 Hz, and their maximum force of contraction was measured. These atria were washed with KR containing 82 mM sodium, and a second PRP was performed 23 min later.

A second group (n=8) was paced at 0.1 Hz in KH and subjected to PRP. They were exposed to 22 μM 2-APB and, after the appearance of SMA, were washed with KH containing 22 μM 2-APB and 82 mM sodium. Superfusion continued for 23 min and a second PRP was performed.

Two groups of left atria determined how 2-APB affects maximum force of contraction in the presence of normal sodium. Here, DIDS was used to block SMA in atria superfused in normal KH.

Control 0.1 Hz paced atria (n=8) were subjected to PRP and exposed to 400 μM DIDS, and a second PRP was performed 23 min later.

A group of atria (n=8) were paced at 0.1 Hz in KH and subjected to PRP. They were exposed to 22 μM 2-APB, 400 μM DIDS was added to the superfusate after the appearance of SMA, and a second PRP was performed 23 min later.

v. Statistical Analyses

Data are the mean±SEM. Fisher least protected significance difference test compared 2 means. Two-way repeated measure analysis of variance compared means between different groups. Significance was assigned at P<0.05.

vi. Materials

KH reagents were from Fisher Scientific (Norcross, Ga.). Choline chloride, sodium glucuronate, and DIDS were from Sigma Chemical (St. Louis, Mo.). 2-APB was from Tocris-Cookson (Ellisville, Mo.).

b. Results

i. 2-APB Induces Spontaneous Mechanical Events in Isolated Rat Left Atria

Initial experiments tested whether 2-APB affects rat left atrial mechanical function. Isolated muscles were superfused and paced at 3 Hz and then exposed to increasing concentrations of 2-APB. All left atria maintained regular mechanical function in the absence of 2-APB. However, intermittent increases in left atrial mechanical force occurred after 5 to 10 min of exposure to >7.5 μM 2-APB. These increases occurred independently of the pacing stimulus (FIG. 8A); and their frequency of occurrence increased with 2-APB concentration (FIG. 8B).

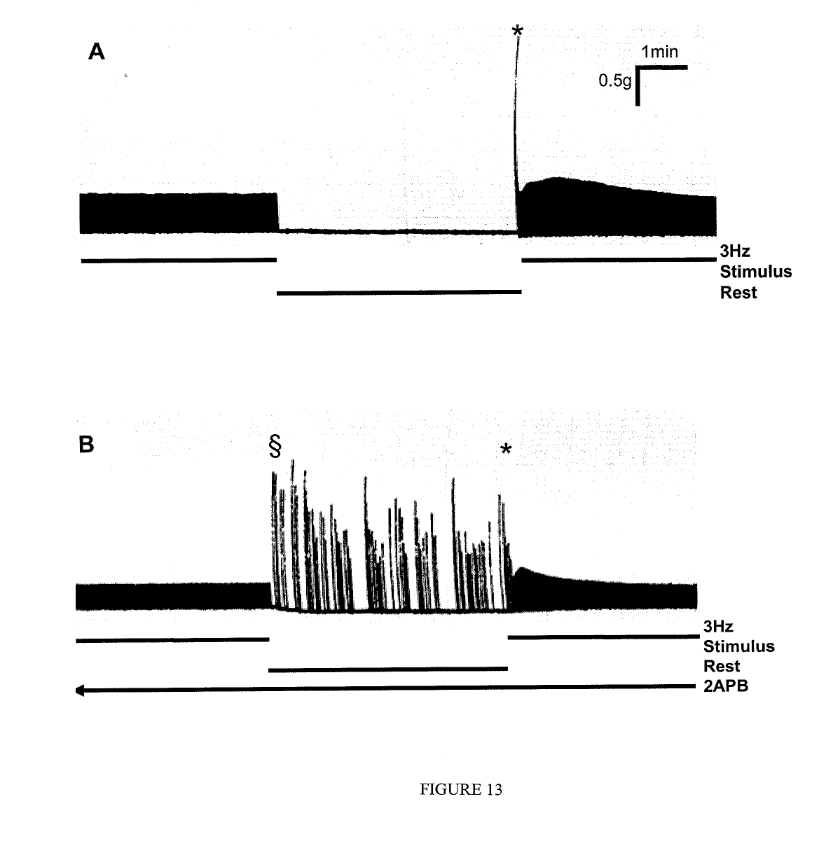

Next tested was whether this instability in regular atrial mechanical function occurred in the absence of a pacing stimulus. Untreated left atria were quiescent in the absence of pacing and showed postrest potentiation of mechanical function (FIG. 13A). In contrast, all atria treated with ≧15 μM 2-APB showed numerous SMEs during rest and a blunted post-rest response (FIG. 13B). SMEs persisted during prolonged rest, occurring continuously for at least 45 min after the end of pacing (data not shown).

SMEs may occur as a result of increases in sub-sarcolemmal calcium arising from a diastolic leak of SR calcium. Thus left atrial pacing rate was lowered to prolong diastole and accentuate any effect that a putative 2-APB-induced calcium leak might have on the stability of regular left atrial mechanical function.

Atria paced at 0.1 Hz showed constant function when superfused in KH, while intermittent SMEs occurred in slowly paced preparations exposed to ≦10 μM 2-APB (FIGS. 9 A and B). Repeated SMEs occurred in atria exposed to >˜15 μM 2-APB, giving way over time to sustained mechanical activity that occurred in the absence of the pacing stimulus, spontaneous mechanical activity (SMA) (FIG. 9C). Both SMA and the mechanical events that occurred in paced, untreated left atria had similar contraction and relaxation times (Table 1).

To evaluate the reversibility of SMA, atria (n=7) were paced at 0.1 Hz, exposed to 15 μM 2-APB, and washed with KH after the appearance of SMA. Removing 2-APB from the superfusate restored regular atrial mechanical function, indicating that SMA is reversible (data not shown).

ii. SMA Depends on the Concentration of Superfusate Sodium, Chloride, and Potassium and is Sensitive to an Anion Transport Inhibitor

It was tested whether altering atrial sodium and chloride gradients affected SMA, as these ions regulate the activity of ion transporters and channels important in initiating ectopic mechanical events. A prolonged diastolic interval was used to highlight the induction or suppression of SMA.

Eight groups of atria were superfused in normal KH, in KH containing decreasing concentrations of sodium at constant chloride and ionic strength, or in KH containing decreasing chloride at constant sodium and ionic strength. All eight groups of left atria were exposed to 15 μM 2-APB for 15 min. SMA occurred in all atria superfused in normal KH (FIGS. 10 and 11; SMA=100% of the total number of preparations). The number of atria exhibiting SMA decreased as superfusate sodium or chloride decreased, so that regular mechanical function was observed in all left atria superfused either in 82 mM sodium or in 30 mM chloride and exposed to 15 μM 2-APB (FIG. 10).

To test whether an anion transport inhibitor could also suppress SMA, atria were superfused in KH, exposed to 2-APB, and were titrated with DIDS while undergoing SMA. At concentrations >300 μM, DIDS reversed SMA completely, restoring regular atrial mechanical function (FIG. 11).

Finally, we evaluated how superfusate potassium affected SMA. Increasing superfusate potassium from 5.8 mM to ˜9.8 mM reversed SMA and restored regular mechanical function (FIG. 7). The concentrations of potassium required to reverse SMA did not affect basal force of contraction in untreated atria (FIG. 7). Both untreated and 2-APB-treated atria became quiescent at ˜14 mM KCl) (FIG. 7).

iii. Evidence that 2-APB May Decrease SR Activator Calcium

Experiments were performed to test whether 2-APB affected the maximum force of atrial contraction, an indicator of the amount of SR activator calcium available for release following depolarization.

The maximum forces of left atrial contraction were not different when measured at the beginning (data not shown) or at the end of a 35-min superfusion in KH, low sodium, or DIDS (FIG. 12). These maximum forces of atrial contraction also were not different from each other (FIG. 12), results indicating that left atria maintained constant and stable function throughout these protocols.

Acutely decreasing superfusate sodium in the presence of constant 2-APB reversed SMA (FIG. 10A). However, the maximum force of left atrial contraction significantly decreased at the end of superfusion in low sodium and 2-APB when compared with values measured before the addition of 2-APB (FIG. 12). DIDS likewise reversed SMA (FIG. 11), and the maximum force of atrial contraction also decreased during superfusion in the presence of 2-APB and DIDS compared with values measured before the addition of 2-APB (FIG. 12). Importantly, the maximum force of contraction measured in (2-APB+DIDS)-treated atria was significantly lower than that measured in (2-APB+low Na)-treated atria (FIG. 12). Thus 2-APB induced greater loss of maximal mechanical function in DIDS-treated compared with low sodium-treated left atria.

2. Confirmation of the Model Against Known Anti-Arrhythmic Agents

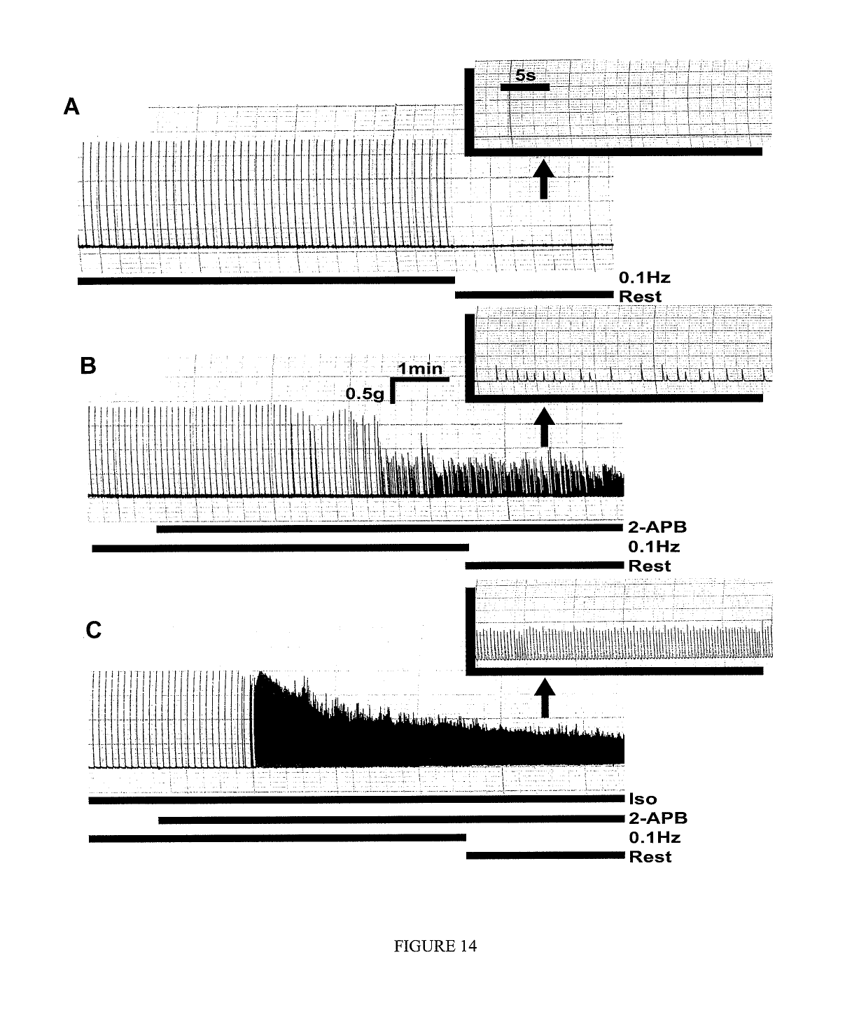

As stated previously, the operation of some embodiments of the model derive from the unexpected discovery that exposing isolated rat left atrial appendage or right ventricular muscle strips to 2-APB induces sporadic ectopic electrical and mechanical events under conditions of normal calcium loading (Wolkowicz et al., J Cardiovascular Pharmacology 49: 325-335 (2007); FIGS. 8 and 13). It was further discovered unexpectedly that, under conditions of increased calcium loading, 2-APB provokes ectopic tachycardic activity in normal rat left atrial appendage and right ventricular muscle strips (Wolkowicz et al., European J Pharmacology 576: 121-131 (2007); FIG. 14C).

This simple model system provokes sporadic and tachycardic ectopy in normal atria and ventricle, providing a new method to test the anti-arrhythmic properties of pharmaceutical agents.

To confirm the validity of the method, embodiments of the method were tested against several known anti-arrhythmic agents.

Ranolazine (N-(2,6-dimethylphenyl)-2-[4-[2-hydroxy-3-(2-methoxyphenoxy) propyl]piperazin-1-yl]acetamide) is a pharmaceutical that is anti-arrhythmic (see the MERLIN human patient trial, Scirica B. et al. Circulation 116: 1647-1652 (2007)). Ranolazine is sold under the trade name “Ranexa” by CV Therapeutics as an anti-anginal medication. On Jan. 31, 2006, ranolazine was approved for use in the United States by the FDA for the treatment of chronic angina. It was tested whether ranolazine suppresses arrhythmic activity.

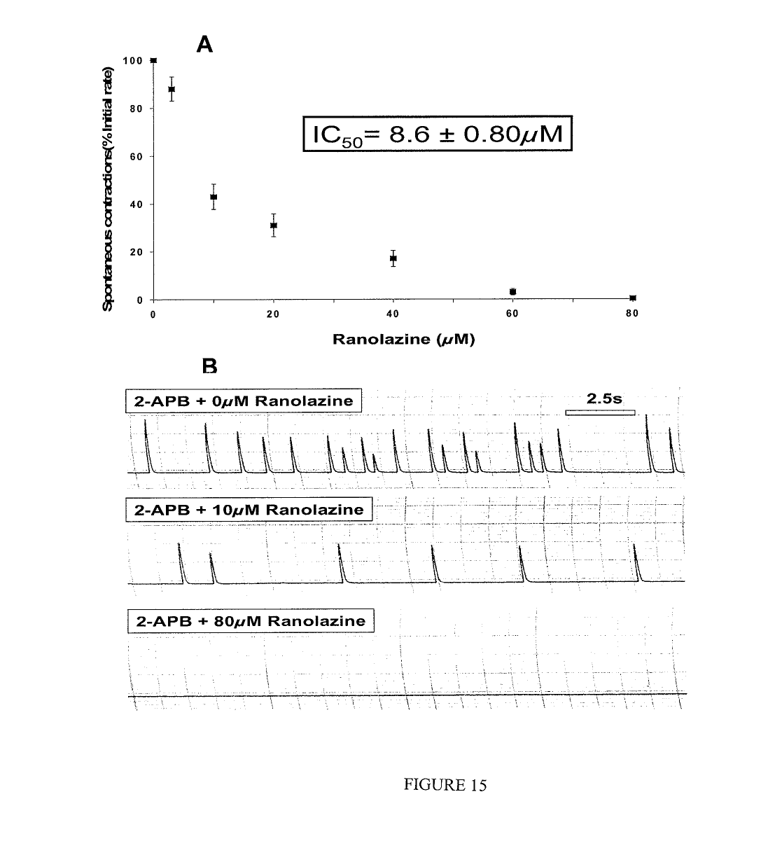

Clinically-relevant concentrations of ranolazine prevent the sporadic ectopic activity in embodiments of the model (FIG. 15A). Ranolazine suppresses, but does not completely prevent, the tachycardic activity that occurs with high calcium loads in this model (data not shown).

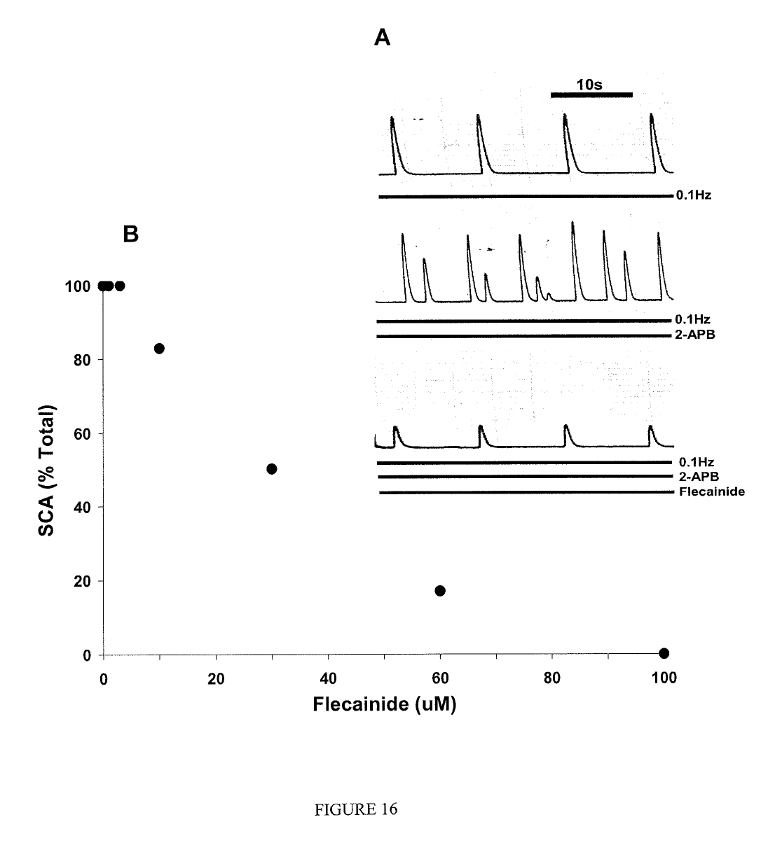

Additional data show that conventional anti-arrhythmic agents like flecainide (FIG. 16) and lidocaine (data not shown) also suppress this arrhythmic activity. Flecamide is an anti-arrhythmic agent sold under the trade names Tambocor, Almarytm, Apocard, Ecrinal, and Flécaine. Lidocaine is a local anesthetic and anti-arrhythmic agent.

These data demonstrate that some embodiments of the assay are capable of positively identifying agents that possess anti-arrhythmic activity in vivo. Taken together, these data support this embodiment of the method as a bioassay for conventional, for novel, and for unknown anti-arrhythmic agents.

3. A Pharmacological Model for Calcium Overload-Induced Tachycardia in Isolated Rat Left Atria

Few experimental models produce spontaneous tachycardia in normal left atria to allow the study of the cellular mechanisms underlying this contributor to atrial fibrillation. The compound 2-APB provokes sporadic SMA and calcium leak in isolated rat left atria. Since sarcoplasmic reticulum calcium leak in the presence of high calcium load may trigger tachyarrhythmias, it was tested how conditions that increase calcium load affect 2-APB-induced ectopic activity. Exposing superfused rat left atria to (i) 30 nM isoproterenol, (ii) 3 μM forskolin, (iii) 300 nM (−)BayK 8644 ((4S)-1,4-Dihydro-2,6-dimethyl-5-nitro-442-(trifluormethyl)phenyl]-3-pyridinecarboxylic acid methyl ester), (iv) 300 nM FPL-64176 (2,5-Dimethyl-4-[2-(phenylmethyl)benzoyl]-1H-pyrrole-3-carboxlic acid methyl ester) or (v) 120 μM ouabain increases their force of contraction, evidence of calcium loading, but does not produce ectopic activity (Table 2). In the context of this example, the term “left atria” encompasses the use of the left atrial appendage. SMA occurs in left atria superfused with 20 μM 2-APB at 47±6 contractions/min in the absence of pacing. Any of these five agents increase rates of 2-APB-induced SMA to >200 contractions/min in the absence of pacing (Table 2). Washing tachycardic left atria with superfusate lacking 2-APB restores normal function, demonstrating the reversibility of these effects (data not shown). Decreasing superfusate sodium and two hyperpolarization-activated current (I_{f}) inhibitors blunt this ectopic activity. Thus conditions that increase atrial calcium load increase the frequency of SMA. Decreasing extracellular sodium and I_{f} inhibitors suppress this spontaneous tachycardia suggesting forward-mode NCX and I_{f}-like activities underlie this activity. This model may help define cell pathways that trigger atrial tachyarrhythmias.

The cellular processes underlying ectopic tachycardia have been investigated using isolated ventricular myocytes as a model system, but few experimental models are available to evaluate spontaneous tachycardia in intact heart muscle, especially left atria, under well-controlled experimental conditions that do not provoke electrical or structural remodeling. This is important since focal left atrial tachycardia is a recognized cause of atrial fibrillation (Jäis et al., 1997). Moderate concentrations of 2-APB produce sporadic SMA in isolated normal rat left atria, and this ectopic activity may arise from sarcoplasmic reticulum calcium leak that stimulates left atria, forward-mode NCX and calcium-activated chloride channel activities (Wolkowicz et al., 2007). Since abnormal myocyte calcium leak in the presence of increased calcium load may generate tachycardic activity in atrium and in ventricle (Tieleman et al., 1997; Pogwizd and Bers, 2004; Tai et al., 2004) it was investigated how conditions that increase atrial calcium load affect 2-APB-induced SMA. Left atria superfused in KH are quiescent in the absence of a pacing stimulus while left atria exposed to ˜20 μM 2-APB produce ˜50 spontaneous contractions per minute in the absence of a pacing stimulus. Alone, either the β-adrenergic agonist isoproterenol or the adenylyl cyclase activator forskolin significantly increase left atrial force and times of contraction but do not affect left atrial mechanical stability (Table 2). That is, mechanical contraction requires an external stimulus in these preparations. In contrast, SMA occurs at >200 contractions/min in the presence of 2-APB and either isoproterenol or forskolin (Table 2); the rate of this spontaneous left atrial tachycardia is significantly greater than the spontaneous contraction rate of untreated rat right atria under our experimental conditions, ˜180 contractions/min. Thus activators of β-adrenergic signaling mediate a transition from SMA to spontaneous tachycardic activity (STA) in isolated normal rat left atria exposed to 2-APB.

β-adrenergic stimulation increases both myocyte calcium load and calcium flux within myocyte compartments (Kamp and Hall, 2000; Bers, 2002). It was tested whether agents that preferentially increase atrial calcium load initiate this novel tachycardic response. Treating left atria with BayK 8644 and FPL-64176 increases their force of contraction presumably because these structurally unrelated slow channel activators increase slow calcium channel open probability (Katoh et al., 2000) (Table 2). Importantly, superfusing left atria with 2-APB and either slow calcium channel activator induces tachycardia within 5 to 10 min (Table 2). Since left atrial relaxation and contraction times during this STA are identical to untreated left atria or to left atria treated with slow calcium channel activators alone (Table 2), either increased left atrial calcium load or increased calcium entry via the slow channel are sufficient to induce tachycardia in the presence of 2-APB.

To differentiate between these two possibilities, rat left atria were treated briefly with a concentration of ouabain sufficient to double their force of contraction (Table 2). Rat cardiac muscle responds poorly to ouabain, nonetheless these increases in contractile force are attributed to an increase in heart calcium load that occurs independently of the slow calcium channel (Illanes and Marshall, 1964; Vassalle and Lin, 2004). Short-term exposure to ouabain alone does not affect rat left atrial mechanical stability; importantly, 2-APB and ouabain induce tachycardia at rates similar to those measured under the preceding four conditions (Table 2). This suggests that an increase in left atrial calcium load in the presence of 2-APB is sufficient to produce STA.

Removing 2-APB from the superfusate suppresses STA suggesting that the cell mechanisms underlying this activity are reversible, at least in the short-term (data not shown). Whether prolonged periods of this novel tachycardic activity change atrial protein function or gene expression to favor remodeling requires further analyses (Tavi et al., 2003; Carnes et al., 2001).

One complication of these results is the fact that rat myocyte calcium handling during excitation-contraction coupling differs from other species; it depends little on sarcolemmal calcium flux (Bers, 2002). In addition, rat sarcoplasmic reticulum has relatively high calcium content under normal conditions (Satoh et al., 1997).

While BayK 8644 and FPL-64176 both increase slow calcium channel open probability, BayK 8644 also interacts with the ryanodine receptor to initiate sarcoplasmic reticulum calcium leak (Katoh et al., 2000); thus ryanodine receptor calcium leak might contribute to this novel tachycardia. This is important as ryanodine receptor calcium leak may elicit ventricular ectopic and tachycardic activity (Marx et al., 2000, Ai et al., 2005). However, as both BayK 8644 and FPL-64176 induce left atrial STA, any effect of the former agent on atrial sarcoplasmic reticulum leakiness may not be important here. In support of this contention, ryanodine decreases left atrial force of contraction, evidence for sarcoplasmic reticulum calcium leakage (Sutko et al., 1997), but does not affect left atrial contraction frequency measured in the presence or absence of BayK 8644 (Table 2).

While a cellular site of action for ryanodine is clearly resolved, how 2-APB influences atrial calcium homeostasis to trigger ectopic activity is less clear. In this regard Maruyama et al., (1997) and Ma et al., (2002), among others (Bootman, 2000), show that low concentrations of 2-APB block IP3R calcium release while moderate concentrations induce leakage of calcium from intracellular stores. Importantly, Ma et al., (2002) reports that 2-APB does not induce calcium leakage in cells lacking IP3 receptors, suggesting that these stores may be the source of leaked calcium proposed to occur under our experimental conditions.

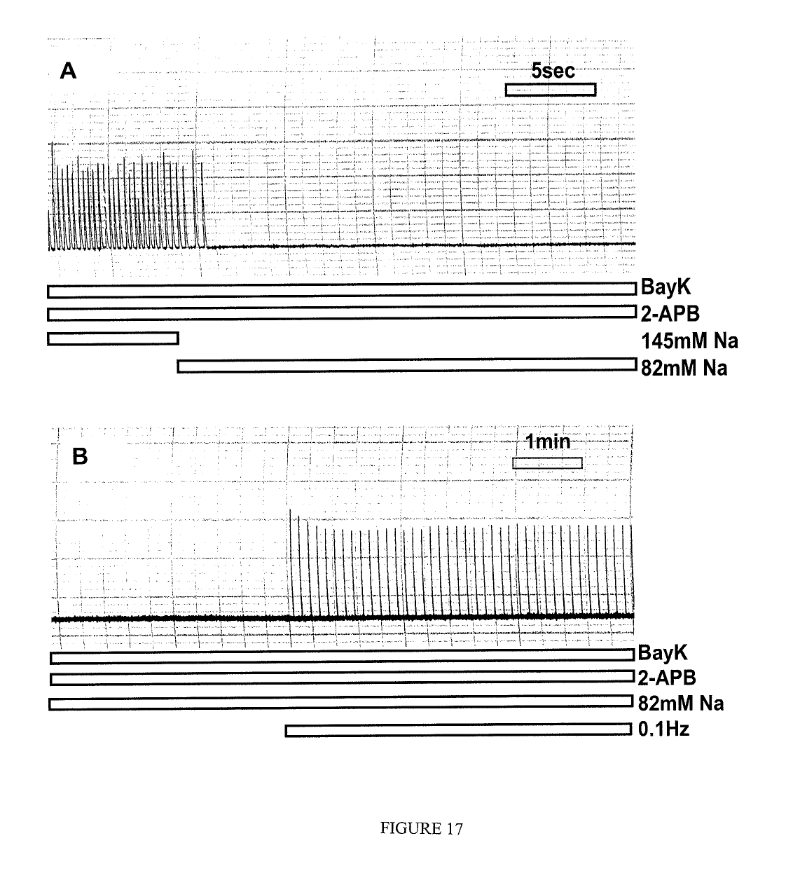

Lowering extracellular sodium suppresses or abolishes phase 4 depolarization in SAN cells, indicating a role for forward-mode NCX in sinoatrial pacemaker activity (Sanders et al., 2006). The data described below show lowering superfusate sodium also suppresses 2-APB-induced left atrial STA promptly and completely (FIG. 17A) perhaps by decreasing the driving force for forward-mode NCX. The ability of an external stimulus to recapture these left atria exposed to low sodium (FIG. 17B) indicates that excitation-contraction coupling remains intact in these isolated muscles. However, lowering extracellular sodium affects multiple ion transport processes that may impact our results; for example, sodium-proton and sodium-bicarbonate exchange may be suppressed here. To critically test whether other consequences of decreased extracellular sodium contributed to the results requires future measurement of atrial pH and resting membrane potential under our conditions. In addition, alterations in sodium current characteristics may contribute both to the ectopic activity described here and to its sodium sensitivity (Belardinelli et al., 2006).

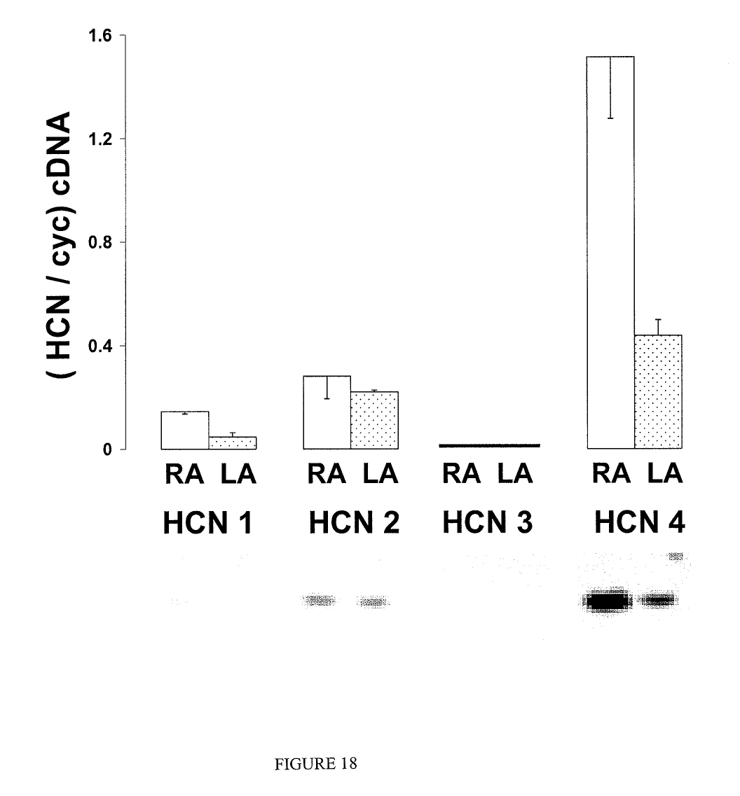

While I_{f}-like activity and HCNs occur in non-sinoatrial myocardium (Carmeliet, 1984; Cerbai and Mugelli, 2006; FIG. 18), previous analyses show that these I_{f} activate outside of a physiologically relevant range of membrane potentials. Nonetheless, activation of non-sinoatrial I_{f} is hypothesized to contribute to ectopic activity (Zorn-Pauly et al., 2004; Cerbai and Mugelli, 2006). Indeed, adenovirus-driven overexpression of HCN2 in dog left atrium produces spontaneous in vivo depolarizations that offer proof-of-principle for this possibility, (Qu, 2003).

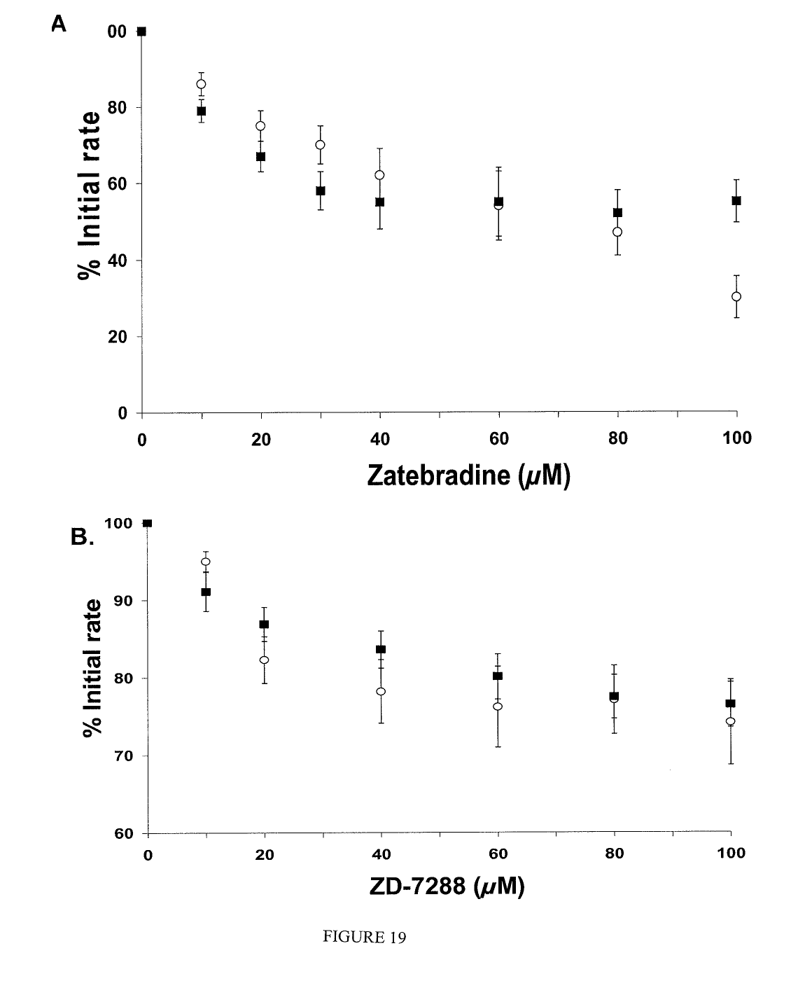

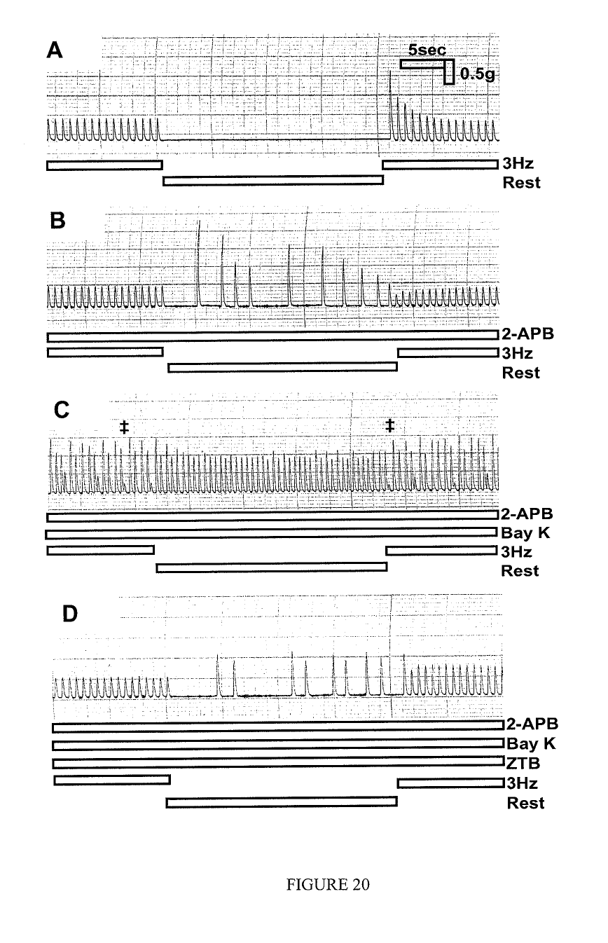

The data described below support the possibility that an endogenous I_{f}-like activity contributes to 2-APB-induced STA. Zatebradine and ZD-7288 (4-Ethylphenylamino-1,2-dimethyl-6-methylaminopyridinium chloride), two structurally unrelated I_{f} blockers, suppress this response (FIGS. 19A and B and FIG. 20). The left atrial I_{f}-like activity observed here appears to be pharmacologically distinct from sinoatrial I_{f}. Specifically, zatebradine inhibits left atrial STA linearly with an IC₅₀ greater than values reported for guinea pig right atria (Perez et al., 1995) or measured in our rat right atria (FIG. 19A). ZD-7288 which binds to the I_{f} channel at a site distinct from zatebradine (Baruscotti et al., 2005) decreases left atrial STA and right atrial spontaneous rates of contraction to a similar degree (FIG. 19B); the latter effect is comparable to that reported in rat right atrial myocytes (Sanders et al., 2006). Furthermore, in contrast to right atrial pacemaker activity where cAMP is required for maximum contraction frequencies, increased calcium load, under conditions that do not affect cyclic nucleotide metabolism (i.e., BayK 8644 or FPL-64176; Katoh et al., 2000), activate left atrial STA maximally. This suggests that cell calcium influences left atrial I_{f}-like activity. Differences in right and left atrial HCN isoform expression (Baruscotti et al., 2005; Cerbai and Mugelli, 2006; FIG. 18), differences in HCN accessory protein expression and post-translational HCN modification, or the expression of HCN splice variants in non-sinoatrial myocardium may underlie the pharmacological and functional differences between right and left atrial spontaneous activity.

Clinical and experimental data indicate that multiple sites of focal tachycardia arise within diseased left atrium including regions near the pulmonary veins and regions of left atrial muscle itself (Jäis et al. 1997; Shah, 2004; Nattel, 2005; Rostock et al., 2006). While reentrant activity may contribute to these clinical observations in diseased atrium (Nattel, 2002), the data described below show that normal left atrial muscle can initiate and sustain spontaneous tachycardia under specific and controllable experimental conditions. Thus, this pharmacological model may help define some of the cell mechanisms responsible for ectopic atrial tachycardia.

a. Materials and Methods

These investigations conform to the Guide for the Care and Use of Laboratory Animals published by the US National Institutes of Health (NIH Publication No. 85-23, revised 1996).

i. Isolation and superfusion of rat left and right atria, and the preparation of 2-APB

Male Sprague-Dawley rats (325-400 g) were anesthetized with intra-peritoneal pentobarbital (0.1 g/kg). Their right and left atria were isolated, superfused at 30° C. in Krebs-Henseleit buffer, and nearly isometric forces of contraction were measured from these muscles (Wolkowicz et al., 2002). 2-APB was prepared as a 150 mM dimethyl sulfoxide stock. Dimethyl sulfoxide did not affect atrial mechanical function.

ii. Effect of β-adrenergic Signaling Activators on Spontaneous Mechanical Activity

Left atria (n=8 per group) were paced at 3 Hz and (i) titrated with 0 to 30 nM isoproterenol (Ahlquist, 1970) or (ii) exposed to 3 μM forskolin (Laurenza et al., 1980). Left atrial force and frequency of contraction, the time to peak tension and relaxation times were recorded after a 3-5 min exposure to forskolin or to any concentration of isoproterenol. The 3 Hz pacing stimulus was interrupted at the end of these protocols to measure SMA.

Left atria (n=10 per group) were paced at 0.1 Hz and treated with (i) 20 μM 2-APB for 10 min, (ii) with 30 nM isoproterenol for 5 min followed by 20 μM 2-APB for 10 min, and (iii) with 3 μM forskolin for 5 min followed by 20 μM 2-APB for 10 min.

Left atrial contraction and relaxation times were recorded at these five times, the pacing stimulus was stopped and rates of SMA were recorded. To determine whether initial pacing rate affects left atrial response to 2-APB and isoproterenol, left atria (n=5-7 per group) were paced at 3 Hz and (i) remained untreated for 15 min, (ii) were treated with 20 μM 2-APB alone for 10 min, or (ii) were treated with 30 nM isoproterenol for 5 min followed by 20 μM 2-APB for 10 min. Atrial mechanical function was measured at these four times; the pacing stimulus then was stopped and rates of SMA were recorded.

iii. Effect of Slow Calcium Channel Activators on SMA

Left atria (n=6 per group) were paced at 3 Hz, titrated with 0 to 300 nM (−)BayK 8644 ((45)-1,4-Dihydro-2,6-dimethyl-5-nitro-4-[2-(trifluormethyl)phenyl]-3-pyridinecarboxylic acid methyl ester; Franckowiak et al. 1985) or FPL-64176 (2,5-Dimethyl-4-[2-(phenylmethyl)benzoyl]-1H-pyrrole-3-carboxlic acid methyl ester); Zheng et al., 1991) and their mechanical function and rates of SMA were recorded after a 5 min exposure to any concentration of slow calcium channel activator.

Left atria (n=8 per group) were paced at 0.1 Hz, exposed to 300 nM BayK 8644 or FPL-64176 for 5 min followed by 20 μM 2-APB for 10 min. Atrial mechanical function was recorded at this time; the pacing stimulus then was stopped and rates of SMA were recorded.

To test whether ryanodine receptor calcium leak produces SMA, left atria (n=6 per group) were paced at 0.1 Hz, treated with 0 or 300 nM BayK 8644 for 5 min, and then exposed to 600 nM ryanodine (Sutko et al., 1997). Atrial mechanical function and rates of ectopic activity were recorded 20 min after exposure to ryanodine.

To evaluate the reversibility of the SMA that occurs in the presence of 2-APB and BayK 8644, left atria (n=6) were paced at 0.1 Hz and exposed to 300 nM BayK 8644 for 5 min and then to 20 μM 2-APB. One minute after the appearance of SMA these left atria were washed with 300 ml (10 bath volumes) of Krebs-Henseleit containing 300 nM BayK 8644. Rates of SMA were measured 5 min later.

iv. Effect of Ouabain on SMA

Left atria (n=7) were paced at 3 Hz, treated with 120 μM ouabain (Illanes and Marshall, 1964), and any change in mechanical function was recorded at a new steady state, ˜10 min later. These atria were exposed to 20 μM 2-APB and their mechanical function and rates of SMA were recorded 10 min later.

v. The Sodium Sensitivity of the SMA Occurring in the Presence of 2-APB and BayK 8644

Rat left atria (n=9) were paced at 0.1 Hz and exposed to 300 nM BayK 8644 and 20 μM 2-APB. Ten min later the pacing stimulus was stopped and 3 min later superfusate sodium was rapidly lowered to 82 mM (Wolkowicz et al., 2007). Changes in the rate of SMA were recorded. Three to 5 min after lowering sodium the 0.1 Hz pacing stimulus was reapplied to recapture these left atria. Superfused rat right atria (n=7) beat at their intrinsic rate in Krebs-Henseleit superfusate for 15 min and superfusate sodium then was rapidly lowered to 82 mM. Right atrial contraction rate was measured at a new steady state, ˜3 min later.

vi. The I_{f} Inhibitor Sensitivity of the STA Occurring in the Presence of 2-APB and BayK 8644

3 Hz-paced left atria (n=9 per group) were superfused (i) in Krebs-Henseleit alone or (ii) they were treated with 20 μM 2-APB for 10 min, (iii) with 300 nM BayK 8644 for 10 min and with 20 μM 2-APB for 10 min, or (iv) they were treated with 300 nM BayK 8644 and 20 μM 2-APB for 10 min, then with 70 μM zatebradine (3-[3-[[2-(3,4-Dimethoxyphenyl)ethyl]methyl amino]propyl]-1,3,4,5-tetrahydro-7,8,-dimethoxy-2H-3-benzazepin-2-one hydrochloride) for 10 min (Baruscotti et al., 2005). Pacing was interrupted at these times and rates of SMA were recorded.

Left atria (n=9) were paced at 0.1 Hz, treated with 300 nM BayK 8644 for 5 min, then with 20 μM 2-APB for 10 min, and the pacing stimulus was stopped. These preparations then were incubated with six increasing concentrations of zatebradine (10-100 μM) for 3-5 min at each concentration, and rates of SMA were recorded. Untreated right atria (n=9) also were titrated with zatebradine and its effect on contraction frequency was recorded.

To test whether zatebradine affects left atrial force of contraction, two groups of left atria (n=9 per group) were paced at 3 Hz and treated with 300 nM BayK 8644. Five minutes later one group was incubated with six increasing concentrations of zatebradine (10-100 μM) for 3-5 min at each concentration, and forces of contraction were recorded. A second group was treated with BayK 8644 but not zatebradine, and forces of contraction were recorded at corresponding times.

To test whether zatebradine affects the SMA occurring in the presence of 2-APB and isoproterenol, left atria (n=7) were paced at 0.1 Hz, treated with 30 nM isoproterenol for 5 min, then with 20 μM 2-APB for 10 min, and the pacing stimulus was stopped. Rates of SMA were recorded, these left atria were exposed to 70 μM zatebradine for 10 min, and rates of SMA were recorded again. A group of right atria (n=7) also were exposed to 30 nM isoproterenol for 5 min and then to 70 μM zatebradine for 10 min. Right atrial contraction frequency was recorded at these two times.

To test whether ZD-7288, an I_{f} inhibitor structurally unrelated to zatebradine (Baruscotti et al., 2005; Sanders et al., 2006) affects the SMA occurring in the presence of 2-APB and BayK 8644, rat left atria (n=7) were treated with 300 nM BayK 8644 for 5 min, then with 20 μM 2-APB for 10 min, and the rates of ectopic activity were recorded. These atria then were incubated with six increasing concentrations of ZD-7288 (10-100 μM) for 3-5 min at each concentration, and the rates of SMA were recorded. Right atria (n=7) were titrated similarly with ZD-7288 (0-100 μM) and the change in their contraction frequency was recorded.

vii. RT-PCR Analysis of Right and Left Atrial Hyperpolarization-activated Cyclic Nucleotide Gated Cation Channel (HCN) mRNAs

Total RNA was isolated from rat left and right atria (n=3 per) using QiaShredder and RNeasy kits. cDNA was transcribed from 1 μg of total left and right atrial RNA using random primers and 200U of reverse transcriptase (Wolkowicz et al., 2004). HCN primers were obtained from the sequences for rat HCN 1 (GenBank accession no. NM053375), rat HCN2 (NM053684), rat HCN3 (NMO53685), and rat HCN4 (NMO21658). Rat cyclophilin was used as an internal control (Wolkowicz et al., 2004). All HCN amplifications were performed using a MJ PTC200 Thermal Cycler (BioRad, 226 Hercules, Calif.) in 500 of Taq PCR Master Mix containing 100 ng of cDNA. Amplifications employed 28 cycles of (i) a 1 min 90° C. denaturing step, (ii) a 45 s 55° C. annealing step, and (iii) a 45 s 72° C. amplification step, and a final 3 min product extension at 72° C. Aliquots of these reactions were electrophoresed through 1.2% agarose gels and analyzed using a Kodak Gel Logic100 imaging system. The intensity of atrial HCN and cyclophilin cDNAs were quantitated using Kodak Molecular Imaging Software.

viii. Statistical Analyses

Data are the mean±S.E.M. Fisher's least protected significance difference test compared two means. Two-way repeated measure analysis of variance compared means between different groups. Significance was assigned at P<0.05.

ix. Materials

Krebs-Henseleit reagents were from Fisher Scientific (Norcross Ga.). (−)BayK 8644, FPL-64176, zatebradine, ZD-7288, ryanodine, and 2-APB were from Tocris-Cookson (Ellisville, Mo.). Forskolin, isoproterenol, and ouabain were from Sigma Chemical (St. Louis, Mo.). QiaShredder, RNeasy RNA isolation kits, and Taq PCR Master Mix were from Qiagen (Valencia, Calif.). Maloney murine leukemia virus reverse transcriptase was from Invitrogen (Carlsbad, Calif.). Oligonucleotides were from MWG Biotech (High Point, N.C.).

b. Results

i. Activators of β-adrenergic Signaling Increase the Frequency of Left Atrial SMA

Isoproterenol and forskolin increase left atrial force of contraction (Table 2), and decrease left atrial time to peak tension and atrial relaxation times (Table 2: cp. Untreated, Iso & Frsk; TPT, T0.5R & TO.9R). Left atria treated with these activators of the β-adrenergic signaling cascade are quiescent in the absence of pacing (Table 2).

2-APB (20 μM) produces SMA in rat left atria at 47±6 contractions/min in the absence of pacing (FIG. 14 and Table 2). The contraction and relaxation times of the contractile events that occur during SMA are similar to values measured in untreated left atria (Tables 1 and 2). Remarkably, SMA occurs at a frequency of 239±11 and 231±5 contractions/min in the presence of 2-APB and isoproterenol or forskolin, respectively (FIG. 14 and Table 2); this high-frequency SMA was designated STA. The time to peak tension and the relaxation times measured in left atria undergoing STA are similar to those measured in atria treated with isoproterenol or forskolin alone (Table 2). Hence activators of β-adrenergic signaling markedly increase the frequency of left atrial STA. This suggests that one or more of the targets or the consequences of adrenergic signaling initiate and maintain STA.

TABLE 2
Pharmacological activation of spontaneous
tachycardic activity (STA)

| | Force | Force | | | | |
|---|---|---|---|---|---|---|
| | Pre | Post | SMA | TPT | T0.5R | T0.9R |
| Condition | (mg/gww) | (mg/gww) | Rate | (msc) | (msc) | (msc) |
| Untreated | 79 | 80 | 0 | 56.2 | 37.4 | 75.0 |
| n = 8 | ±7 | ±6 | | ±0.8 | ±1.7 | ±2.5 |
| 2APB | — | — | 47^{‡} | 57.5 | 35.9 | 68.4 |
| n = 10 | | | ±6 | ±1.1 | ±0.8 | ±1.7 |
| Iso | 31 | 104* | 0 | 45.0^{‡} | 29.3^{‡} | 58.7^{‡} |
| n = 8 | ±8 | ±5 | | ±0.9 | ±0.7 | ±1.4 |
| Iso + 2APB | — | — | 239^{§} | 45.4 | 29.6 | 57.1 |
| n = 10 | | | ±11 | ±1.1 | ±0.4 | ±1.8 |
| Forskolin | 21 | 62* | 0 | 44.6* | 28.7^{‡} | 58.4^{‡} |
| n = 8 | ±5 | ±8 | | ±0.8 | ±0.9 | ±2.6 |
| Forskolin + | — | — | 231^{§} | 47.2 | 29.0 | 54.0 |
| 2APB | | | | | | |
| n = 10 | | | ±6 | ±0.9 | ±0.9 | ±1.9 |
| Bay K | 33 | 83* | 0 | 54.1 | 35.2 | 73.2 |
| n = 6 | ±3 | ±6 | | ±1.0 | ±0.7 | ±1.4 |
| BayK + 2APB | — | — | 227^{§} | 55.8 | 36.5 | 71.8 |
| n = 8 | | | ±10 | ±1.0 | ±0.8 | ±2.3 |
| FPL | 30 | 68* | 0 | 52.8 | 35.3 | 73.3 |
| n = 6 | ±7 | ±5 | | ±1.0 | ±3.3 | ±3.2 |
| FPL + 2APB | — | — | 222^{§} | 58.3 | 37.1 | 72.5 |
| n = 8 | | | ±9 | ±1.7 | ±1.4 | ±2.5 |
| BayK + Ryndn | 67 | 17* | 0 | 55.0 | 36.3 | 67.5 |
| n = 6 | ±7 | ±3 | | ±0.5 | ±1.2 | ±1.4 |

“Condition” = experimental condition; “Iso” = 30 nM isoproterenol; “Forskolin” = 3 μM forskolin; “2APB” = 20 μM 2-APB; “Bay K” = 300 nM (−)Bay K 8644; “FPL” = 300 nM FPL-64176; “Ryndn” = 600 nM ryanodine; “Ouab” = 120 μM ouabain; “Pace” = pacing rate; n = number of independent measures; “Force” = mg force per g atrial wet weight (gww), prior to (Pre) or following (Post) a 10 min exposure to the agent; “SMA Rate” = spontaneous atrial mechanical events (SMEs) per minute; “TPT” = time to peak tension; “T0.5R” = time to 50% relaxation; “T0.9R” = time to 90% relaxation.
Data are mean ± SEM.
*= p < 0.05 vs. Pre-treatment force;
^{‡}= p < 0.05 vs. Untreated;
^{§}= p < 0.05 vs. corresponding control.

STA does not depend on the initial rate of atrial pacing. Specifically, rapid ectopic activity arose immediately following the termination of 3 Hz pacing in left atria that were exposed to isoproterenol and 2-APB (FIG. 20C). Mechanical discordance occurs in [isoproterenol+2-APB]-treated left atria when paced at 3 Hz and dissipates in the absence of pacing (FIGS. 14C and 20C: cp. 3 Hz & Rest).

ii. Slow Calcium Channel Activators Induce STA

BayK 8644 and FPL-64176 increase left atrial force of contraction (Table 2) without affecting the time to peak tension or left atrial relaxation times (Table 2), indicating that left atrial calcium loading takes place here. SMA or STA does not occur in left atria treated with either slow calcium channel activator alone (Table 2).

STA occurs at 227±10 and at 222±9 contractions/min in left atria treated with 2-APB and 300 nM BayK 8644 or FPL-64176 (Table 2). These rates are not different from those measured in left atria treated with 2-APB and isoproterenol or forskolin. However, time to peak atrial tension and the relaxation times measured in left atria treated with 2-APB and either slow calcium channel activator are similar to those measured in untreated preparations (Table 2). Thus, appendage STA can occur in the absence of any significant change in contraction or relaxation time.

Washing spontaneously tachycardic left atria with superfusate containing slow calcium channel activator but no 2-APB completely reverses this tachycardic response (224±12 vs. 0.7±0.4 contractions/min, Pre- and Post-washing, respectively). This suggests that a short-term exposure to the experimental conditions that elicit spontaneous tachycardia does not disrupt left atrial integrity.

Next was tested whether ryanodine receptor calcium leakage could produce similar types of ectopic activity. Time-dependent decreases in force of contraction occur in left atria exposed either to 600 nM ryanodine or to BayK 8644 and ryanodine, indicating leakage of sarcoplasmic reticulum activator calcium (Table 2). However, no STA occurs under these conditions (Table 2). This suggests that while ryanodine receptor calcium leak depletes sarcoplasmic reticulum calcium stores and may trigger ectopic events under some conditions (Marx et al. 2000; Ai et al., 2005), it does not contribute to the activity reported here.

iii. Ouabain Induces STA

Short-term exposure of superfused 3 Hz-paced rat left atria to 120 μM ouabain doubles their force of contraction, an indication of calcium loading (Vassalle and Lin, 2004), but does not affect their mechanical stability (Table 2). A subsequent 5-10 min exposure to 2-APB produces STA at 202±5.6 contractions/min (Table 2). Similar time to peak tension and left atrial relaxation times occur in ouabain- and (ouabain+2-APB)-treated left atria, and are comparable to untreated preparations (Table 2).

Thus left atrial STA occurs with five experimental conditions that increase calcium load via distinct mechanisms; β-adrenergic signaling, slow calcium channel activation, and ouabain inotropy (Kamp and Hall 2000; Kutch et al., 2000; Vassalle and Lin, 2004).

iv. STA is Sensitive to Superfusate Sodium

Rapidly lowering superfusate sodium from 145 to 82 mM abruptly suppresses left atrial STA and left atria remain quiescent under these conditions (FIG. 17). Excitation-contraction coupling remains functionally intact in these preparations as a 0.1 Hz pacing stimulus recaptures them (FIG. 17). Rat right atrial contraction frequency also decreases significantly when superfusate sodium is reduced to a similar extent (145 mM Na=163±11.8 vs. 82 mM Na=65±7.1 contractions/min) (Ju and Allen, 1998; Hünser et al., 2000; Sanders et al., 2006).

v. I_{f} Inhibitors Suppress STA

Zatebradine (70 μM) significantly decreases both the rate of STA in left atria treated with BayK 8644, and 2-APB (FIG. 19A) and the mechanical discordance that occurs in 3 Hz-paced left atria treated with BayK 8644 and 2-APB (FIG. 20D). Zatebradine decreases STA linearly with an IC₅₀ of 58±5 μM in intact left atria exposed to BayK 8644 and 2-APB (FIG. 19A). This I_{f} inhibitor also decreases the rate of appendage STA in left atria treated with isoproterenol and 2-APB (225±3.6 vs. 110±24.5 contractions/min; (isoproterenol +2-APB) vs. (isoproterenol+2-APB+70 μM zatebradine), respectively). Zatebradine decreases right atrial contraction frequency to a maximum of ˜50% with an IC₅₀ of 12±1.1 μM (FIG. 19A), values similar to those reported for guinea pig right atria (Perez et al., 1995). Isoproterenol increases right atrial contraction frequency (177±4.7 vs. 276±11.4 contractions/min; 0 vs. 30 nM isoproterenol, respectively) and zatebradine depresses right atrial contraction frequency under these conditions as well (212±9.7 contractions/min; (isoproterenol +70 μM zatebradine)).

Zatebradine is structurally related to the slow calcium channel antagonist verapamil (Doerr and Traunvein, 1990). To assure that zatebradine does not suppress left atrial slow channel activity under our conditions, we measured the force of contraction of left atria that were treated with BayK 8644 and then (i) titrated with 10-100 μM zatebradine or (ii) were left untreated. Zatebradine does not affect left atrial force of contraction under these conditions suggesting that it does not inhibit slow calcium channel activity across this range of concentrations (Untreated=97±1.5% vs. Zatebradine-treated=95±3.8% of initial force).

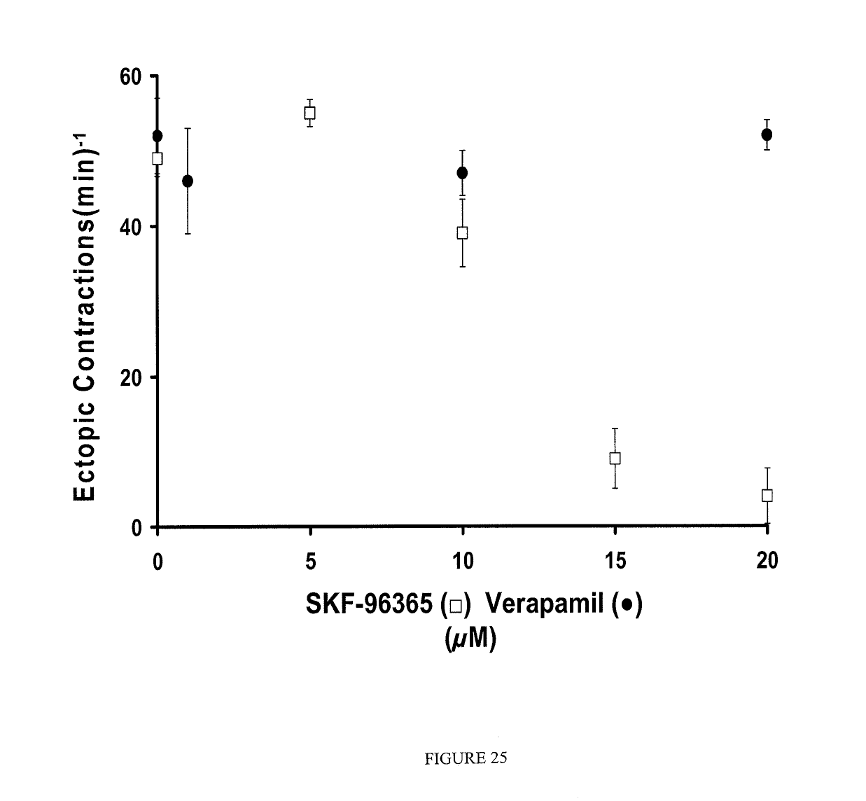

ZD-7288, an I_{f} inhibitor structurally unrelated to zatebradine, also decreases left atrial STA initiated by BayK 8644 and 2-APB (FIG. 25B: ▪). As expected (Sanders et al., 2006), ZD-7288 decreases right atrial contraction frequency less robustly than zatebradine (FIG. 19B).

vi. Rat Left Atria Contain HCN mRNAs

Rat left atria contain HCN2 mRNA in amounts similar to those measured in rat right atria (FIG. 18) and HCN4 mRNA in lower amounts than those in right atria (FIG. 18). Rat left atria contain no detectable HCN3 (FIG. 18) and little HCN I mRNA (FIG. 18).

4. Ventricular Muscle Models of SMA and STA

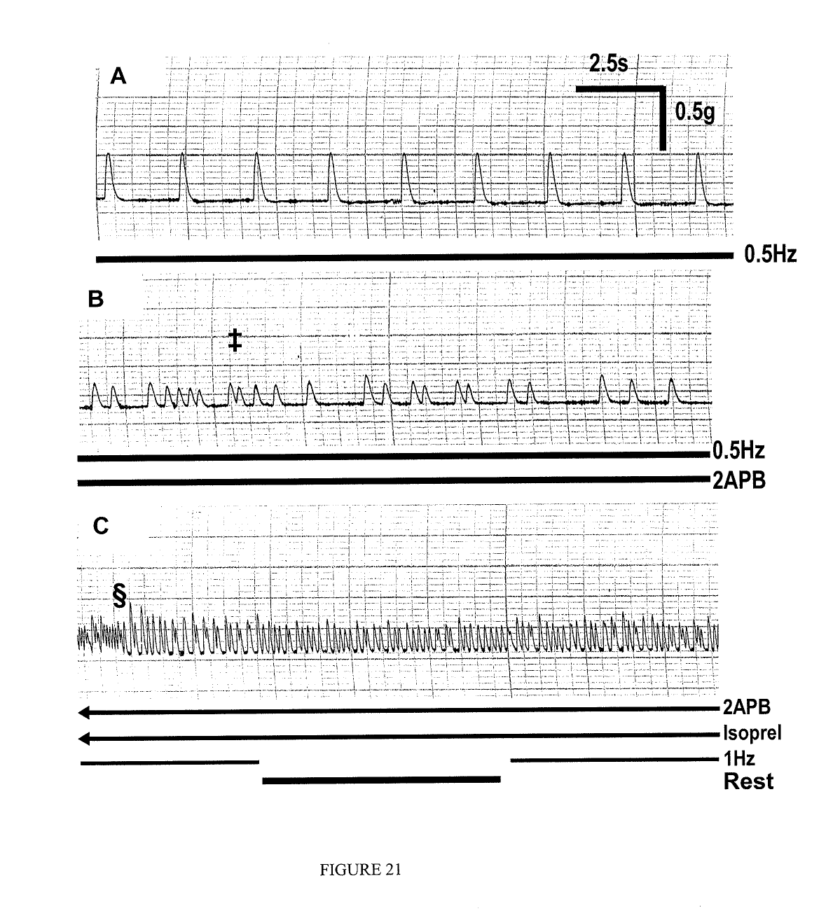

To further establish this model as a general test for arrhythmic and anti-arrhythmic agents, right ventricular muscle strips and right ventricular papillary muscles were isolated from anesthetized rats using methods known to persons skilled in the art. These muscles were then superfused as described above and paced at 0.5 Hz. These muscles show normal excitation-contraction coupling and produce mechanical force only when stimulated (FIG. 21A). When 2-APB or a structurally or functionally related compound such as diphenyl boronic anhydride (DPBA) was added to the superfusate in appropriate amounts, ventricular SMA was observed (FIG. 21B). This ventricular muscle showed STA, similar to atrial muscle, when it is exposed to an agent that enhances heart muscle calcium stores (FIG. 21C).

Thus all non-automatic cardiac muscle tested shows the ability to express both sporadic and tachycardic electro-mechanical activity under the experimental conditions described herein. As it has been shown that atria and ventricle contain distinct ion channel characteristics, the model described here can be used to identify anti-arrhythmic agents that may be potentially specific to atrial muscle, to ventricular muscle, or to both.

5. Model Simulating Ventricular and Atrial Fibrillation

This example demonstrates that the model systems described may be used to screen for agents that suppress atrial or ventricular fibrillation and tachycardia. The example confirms that tachycardia and fibrillations can be simulated using the model.

It has been observed that bioassay temperature is preferably increased to 37° C. This is advantageous due to the difference in the activation energy profile between normal SAN activity and this ectopic activity.

To determine the relationship between temperature and rates of SMA, rat left atrial appendage (n=6-8) were superfused at 30° C., paced at 0.1 Hz, treated for 5 min with 300 nM (−) BayK 8644 and then treated for 10 min with 22 μM 2-APB. Pacing was stopped and rates of STA were recorded. A second group of appendages (n=8) were superfused at 23° C. and treated identically to the first. After 10 min STA was measured without pacing and muscle bath temperature was increased to 37° C. Maximum spontaneous contraction rates were measured over 10 min.

Rat right atria (n=8) were superfused at 30° C. and rates of SAN pacemaker-driven mechanical contraction were recorded. A second group of right atria was superfused at 23° C. and their spontaneous contraction rates were recorded; muscle bath temperature was increased to 37° C. and maximum rates of spontaneous contraction were measured over 10 min.

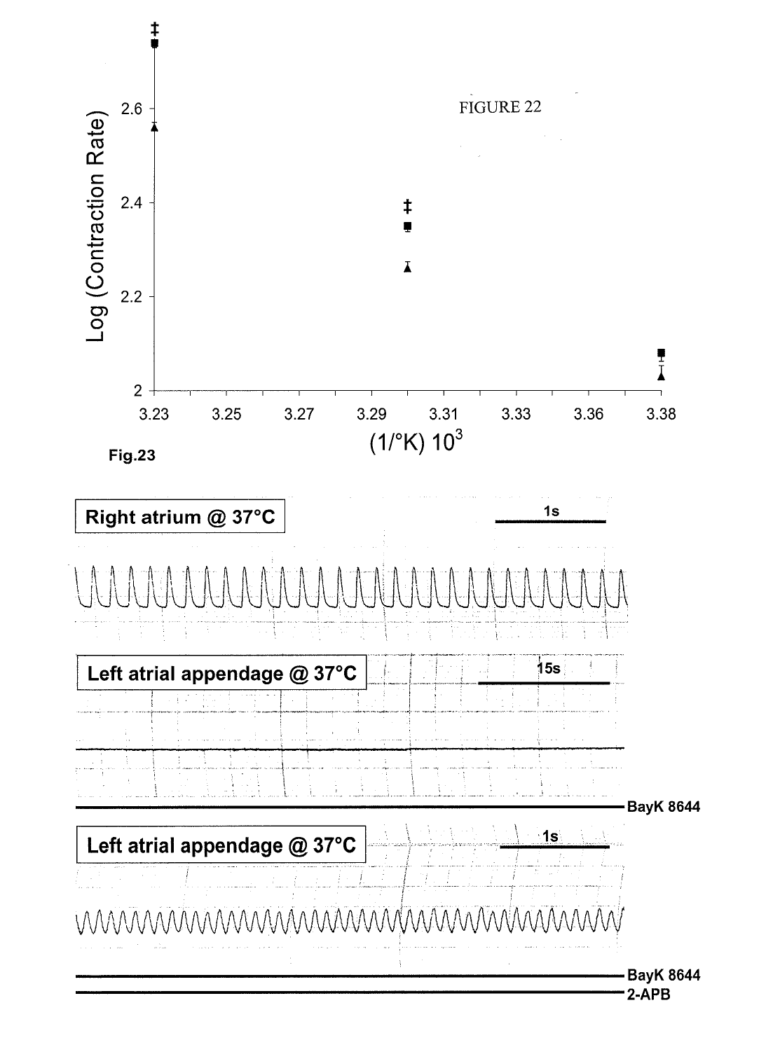

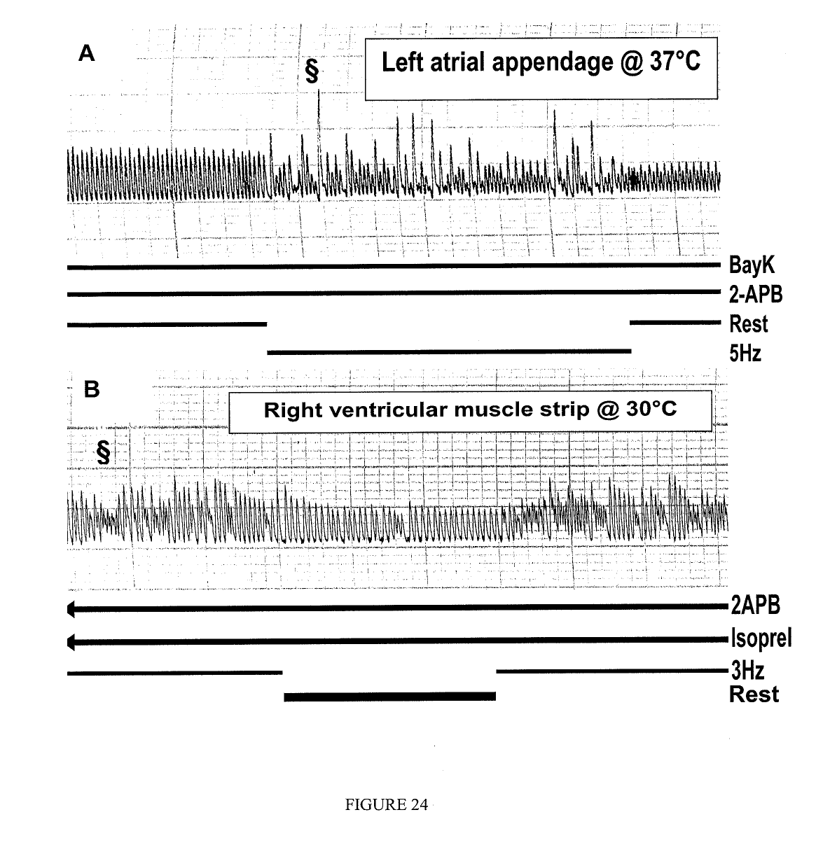

As shown in FIG. 22, a predictable relationship was discovered between temperature and contraction rate. FIG. 23 shows mechanical function of isolated cardiac muscle at 37° C. The upper graph shows mechanical function of a superfused rat right atrium measured without pacing at 37° C. The middle graph shows mechanical function of a 0.1 Hz-paced, superfused left atrial appendage treated for 5 min with 300 nM (−)BayK 8644 alone at 37° C. without pacing. The lower graph shows mechanical function of a rat left atrial appendage superfused at 37° C. with 300 nM BayK 8644 and 20 μM 2-APB, and measured without pacing.

While fibrillation-like results can be obtained at 30° C., increasing the bio-assay temperature to 37° C. increases the influence this ectopic activity has over the electromechanical instability of isolated heart muscle.

Clinical evidence suggests that human atrial and ventricular fibrillation arise when one or more ectopic tachycardic foci continually discharge and disrupt normal heart electromechanical function driven by the SAN. Further experiments demonstrated that the model recapitulates this scenario in isolated atrial or ventricular muscle. Specifically, isolated, superfused heart muscle, atrial or ventricular, treated with an appropriate concentration of 2-APB (>20 μM) and with an agent that increases myocyte calcium load was exposed to external pacing that stimulates the muscle at a physiological rate and with strength just sufficient to capture it. Under these conditions isolated atrial and ventricular muscle produced disorganized, fibrillation-like mechanical activity (FIG. 24). In the absence of external pacing atrial and ventricular muscle spontaneously contracted (FIGS. 24 and 21C, Rest).

This regular tachycardic activity indicates the persistence of the underlying arrhythmic principle responsible for the fibrillation-like activity in embodiments of this model.

Thus, the above experiments establish the operation of one embodiment of this

bioassay as follows: 1) Isolated left atrial appendage or ventricular muscle superfused or perfused in Krebs-Henseliet perfusate at a physiological temperature paced initially at a slow sub-physiological rate; 2) Exposure of this muscle to >20 μM of 2-APB or a structurally- or functionally-related compound; 3) Exposure of this muscle to a condition that increases its internal calcium either prior to step (2) or following the appearance of ectopy in the presence of 2-APB; for example, isoproterenol (˜30 nM) or calcium channel activators (−)BayK 8644 (300 nM); 4) Increase pacing to an appropriate physiology rate (˜5-6 Hz in the case of Norway rat muscle) following the appearance of ectopic tachycardia; 5) Treat with known or novel anti-arrhythmic agents to restore the fibrillating muscle to a condition in which it contracts in strict unison with the external stimulus.

### EXAMPLES

### Example 1

### SKF-96365, But Not Verapamil, Prevents Triggered Activity

The present disclosure provides for the use of SKF-96365 as a novel anti-arrhythmic agent. In order to evaluate SKF-96365 as a potential anti-arrhythmic agent, the effect of SKF-96365 on triggered activity was evaluated. As discussed above, triggered activity results from the abnormal generation of electrical activity in regions of the heart other than the SAN of the right atrium. The SAN is the origin of the spontaneous electrical activity that drives normal rhythmic atrial and ventricular contractions. Abnormal triggered (i.e., ectopic) electrical activity is thought to arise, at least in part, from altered calcium homeostasis within heart muscle cells themselves, although other mechanisms may contribute as well. Triggered activity occurs through two mechanisms; afterdepolarizations and abnormal automaticity.

In this example, it was tested whether SKF-96365 can reverse triggered activity. In these experiments, an ATX II model system was used to simulate triggered activity. Specifically, superfused, 1 Hz-paced left atria (n=5-7) were treated with 25 nM ATX II to initiate triggered activity. Following the appearance of triggered activity, the superfusate was titrated with increasing concentrations of SKF-96365 (5 to 20 μM). Triggered activity contractions were recorded after about 5 min of incubation at each drug concentration. SKF-96365 produced a concentration-dependent decrease in triggered events induced by ATX II with an IC₅₀ of about 12 μM (FIG. 25).

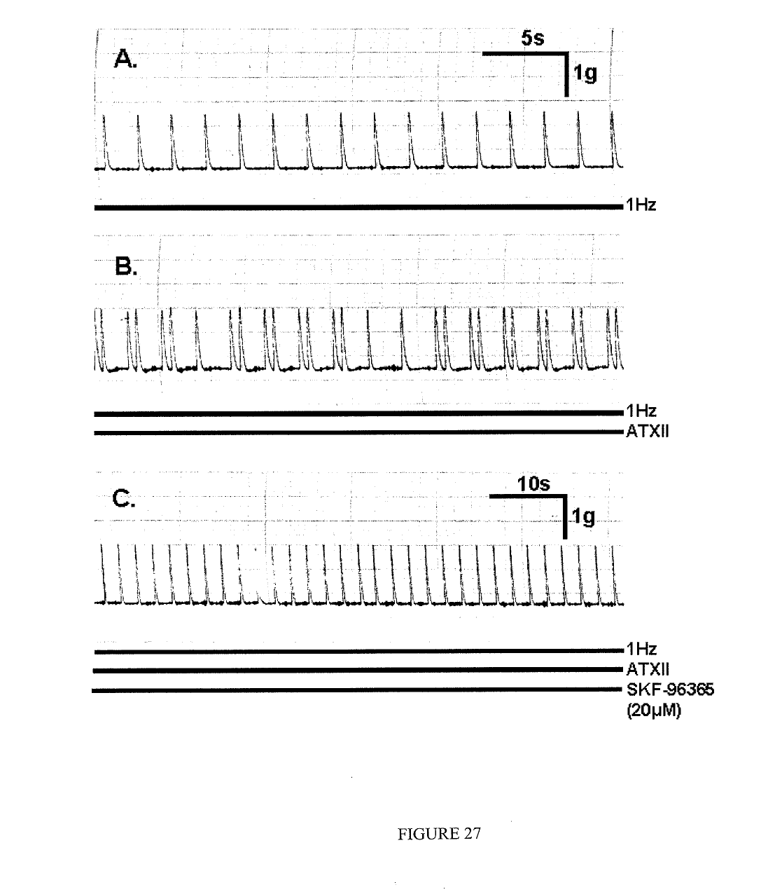

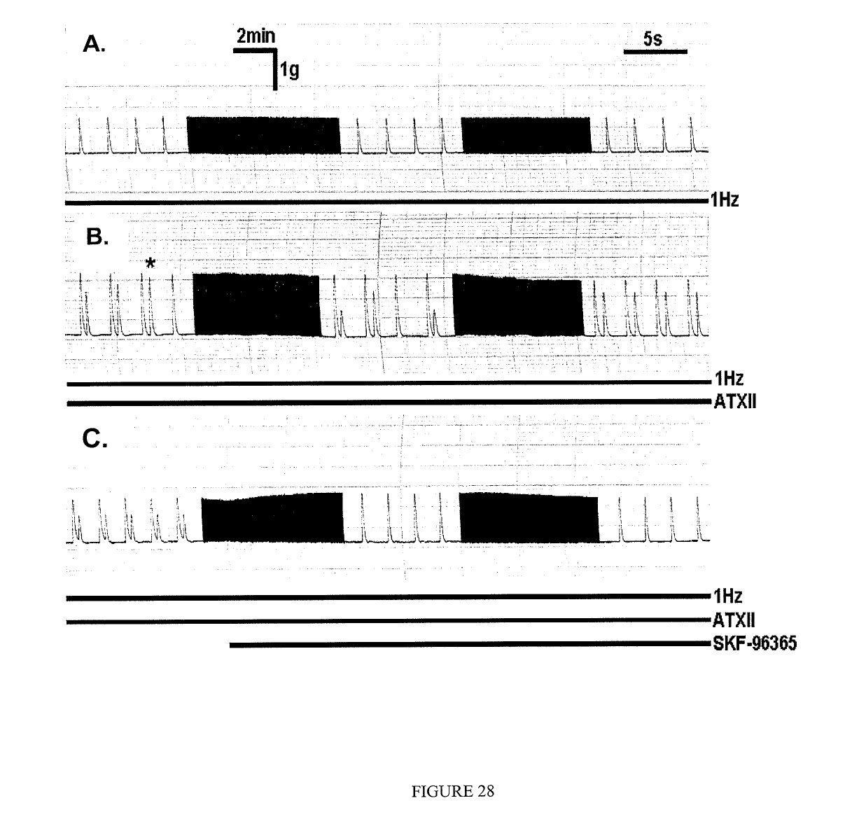

The data in FIGS. 27 and 28 further confirm the results of FIG. 25. FIG. 27A shows the normal contraction pattern of left atria paced at 1 Hz. This non-automatic muscle contracts only when stimulated. When treated with 25 nM ATX II, triggered activity is induced as indicated by contractile doublets following a single electrical stimulus (FIG. 27B). After exposure to 20 μM SKF-96365 for 5 min, the muscle shows no triggered activity following 1 Hz pacing stimulus (FIG. 27C). Likewise, FIG. 28A shows the normal contraction pattern of left atria paced at 1 Hz. When this non-automatic muscle is treated with 25 nM ATX II, triggered activity is induced as indicated by contractile doublets following a single electrical stimulus (FIG. 28B). Again, the addition of SKF-96365 at 20 μM eliminates triggered activity over a few minutes time (FIG. 28C; SKF-96365).

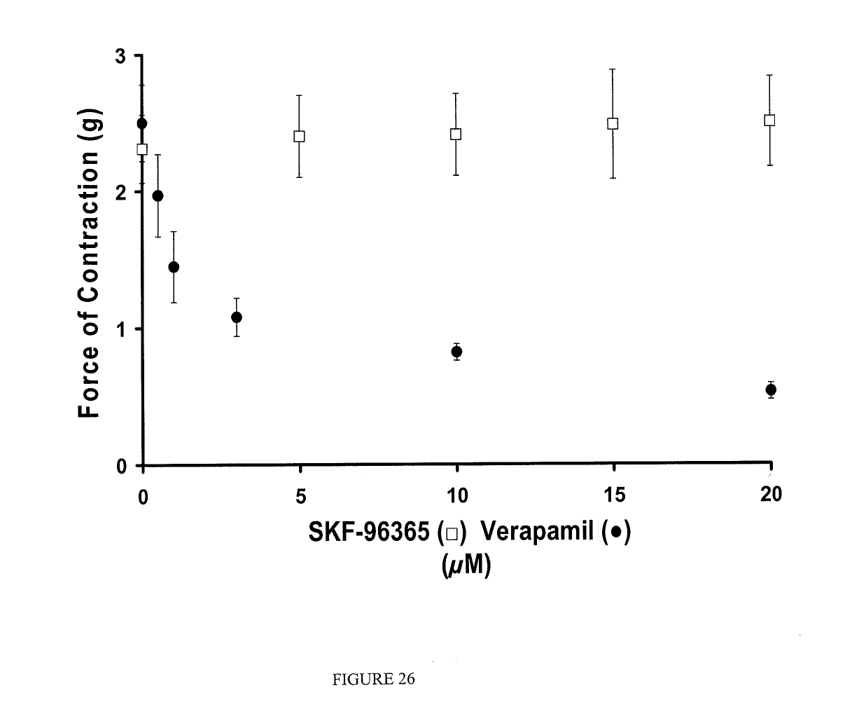

Furthermore, SKF-96365 did not decrease the mechanical performance of these left atria, indicating that normal excitation-contraction coupling was not affected by this compound (e.g., FIG. 26, FIG. 28C). This was an unexpected result as many compounds decrease mechanical performance of the left atria in addition to inhibiting triggered events in this model. In addition, the effect of SKF-96365 on the reversal of triggered activity can be affected acutely. Adding SKF-96365 to the superfusate of left atria undergoing triggered activity reverses this activity within 2-5 min (FIG. 28C)

### Example 2

### SKF-96365 and Verapamil Exhibit Different Effects on Triggered Activity

In this example, it was tested whether SKF-96365 can reverse triggered activity. In these experiments, an ATX II model system was used to simulate triggered activity. Specifically, superfused, 1 Hz-paced left atria (n=5-7) were treated with 25 nM ATX II to initiate triggered activity.

The ability of SKF-96365 to reverse triggered activity was compared to that of verapamil, a canonical voltage-dependent calcium channel blocker. In these experiments, triggered contractions were induced by ATX II as in Example 1. Following the appearance of triggered activity, the superfusate was titrated with increasing concentrations of verapamil (2 to 20 μM). Triggered activity contractions were recorded after ˜5 min of incubation at each drug concentration. In contrast to SKF-96365, verapamil did not affect the rate of triggered activity in these left atria (FIG. 25)

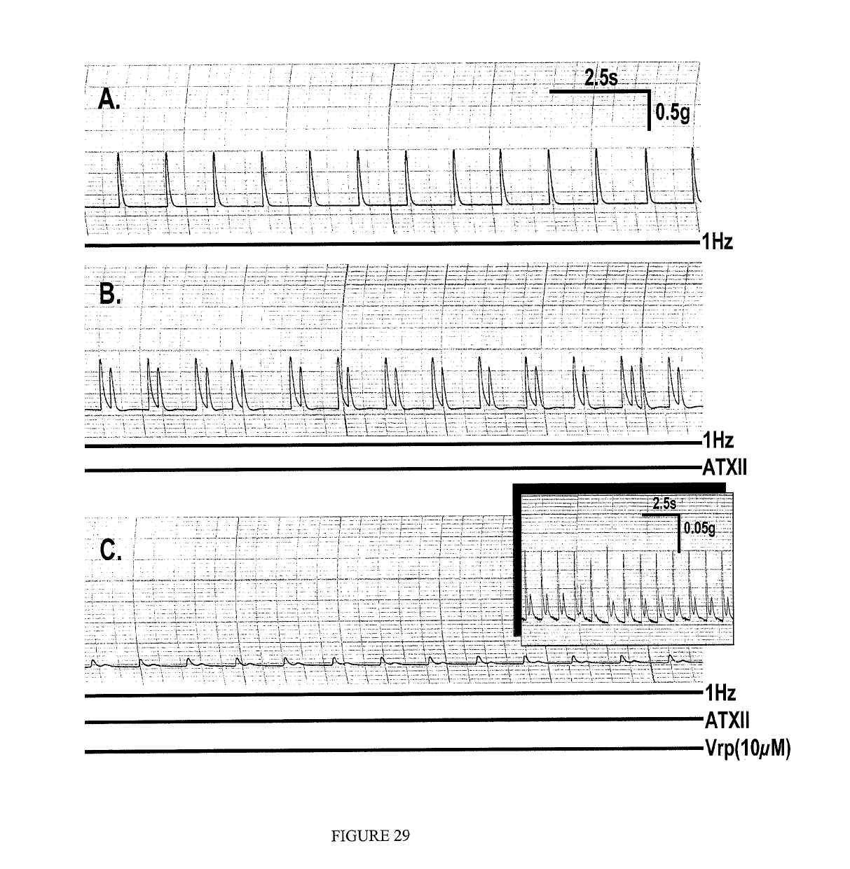

Verapamil negatively impacted the force of contraction of superfused left atria treated with ATX II. 1 Hz-paced left atria (n=5-7) were treated with 25 nM ATX II. Following the appearance of triggered activity, the superfusate was titrated with increasing concentrations of verapamil (2-20 μM). Force of contraction was recorded after 5 minutes of incubation at each drug concentration. In contrast to SKF-96365, verapamil markedly decreased contractile force most likely because of its ability to block slow calcium channel activity and calcium-induced calcium release (FIGS. 26 and 29).

The data in FIG. 29 further confirm the results of FIGS. 25 and 26. FIG. 29A shows the normal contraction pattern of left atria (treated as described above) paced at 1 Hz (panel A). This non-automatic muscle contracts only when stimulated. When treated with 25 nM ATX II, triggered activity is induced as indicated by contractile doublets following a single electrical stimulus (FIG. 29B). After exposure to 10 μM verapamil for 5 min, the muscle shows markedly depressed mechanical function (c.p., FIG. 29C to FIG. 27C or 32C). However, if the scale of FIG. 29C is expanded (Inset), the continued presence of triggered events is clearly seen.

The foregoing data show that verapamil suppresses mechanical function but not triggered activity. As expected, verapamil severely decreased left atrial mechanical function most likely because it inhibited the voltage-dependent calcium entry that initiates SR calcium-induced calcium release and left atrial contraction.

Therefore, SKF-96365 and verapamil have two different effects on triggered activity and left atrial mechanical function. The former suppresses triggered activity but not mechanical function; the latter does not affect triggered activity but depresses left atrial force of contraction.

### Example 3

### SKF-96365 Prevents Triggered Activity

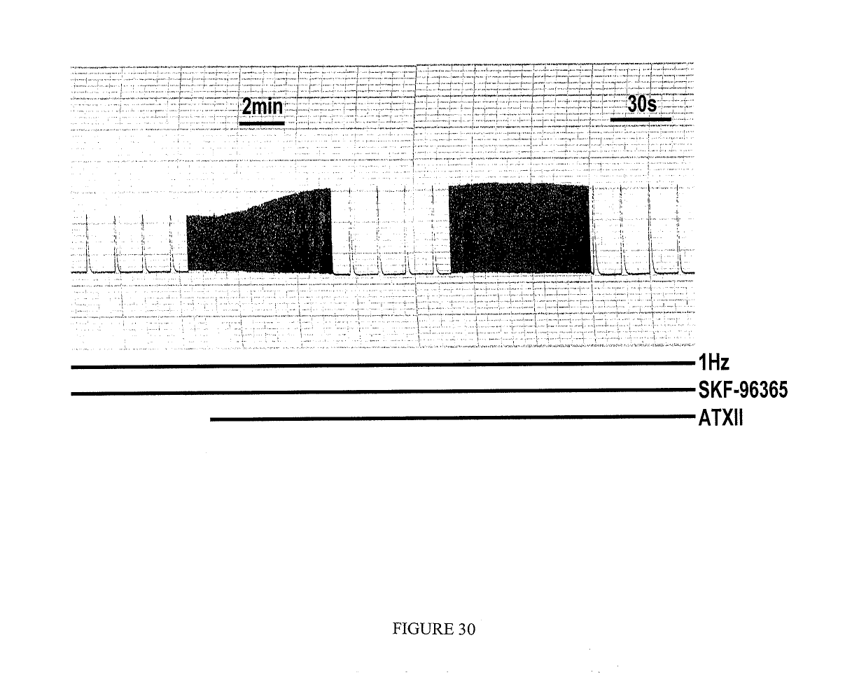

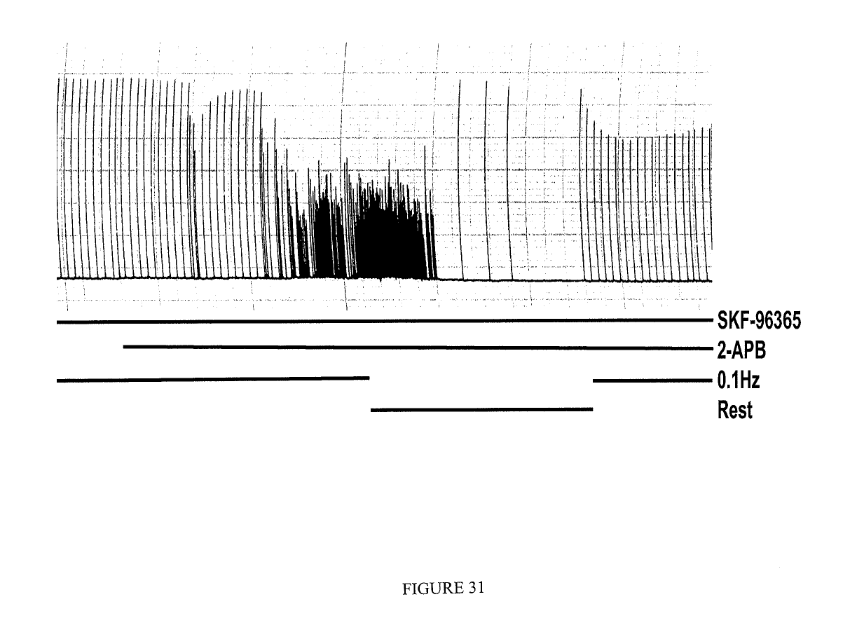

In this example, it was evaluated whether SKF-96365 can prevent triggered activity. In these experiments, an ATX II model system was used to simulate triggered activity. Specifically, superfused left atria were treated with SKF-96365 (20 μM) for 10 min then exposed to 25 nM ATX II and the appearance of triggered mechanical events was recorded over a 10-15 min time period. SKF-96365 pre-treatment prevents the appearance of triggered activity (FIG. 30). An inotropic response occurred following ATX II addition, likely due to calcium loading. However, triggered activity did not occur as this muscle responds solely to the external pacing stimulus with a single contraction.

### Example 4

### SKF-96365 Reverses Abnormal Automaticity (SMA/STA)

In this example, it was evaluated whether SKF-96365 could reverse abnormal automaticity. In these experiments, a 2-APB model system was used to simulate abnormal automaticity. Specifically, paced (0.1 Hz) superfused left atria were treated with 2-APB (20 μM) and the pacing stimulus was stopped after the appearance of SMA. SKF-96365 (25 μM) was then added to the superfusate and the effect on SMA was measured. Exposing left atria to 25 μM SKF-96365 suppressed SMA within 2-3 min and the muscle became quiescent in the absence of pacing (FIG. 31). SKF-96365 did not affect normal excitation-contraction coupling as restoring the 0.1 Hz pacing stimulus provoked mechanical activity similar in magnitude to untreated left atria which occurred only following a pacing stimulus (FIG. 31; right side of panel).

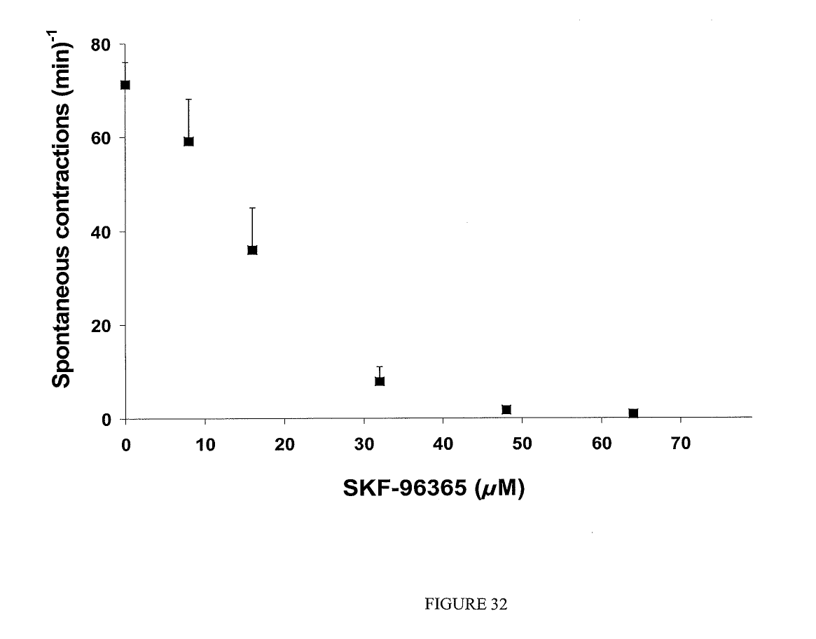

Additional experiments determined the concentration-dependence of SKF-96365 inhibtion of SMA. These experiments were performed as described above except that various concentrations of SKF-96365 were added to the left atrial superfusate (about 10-65 μM). Exposing left atria to various concentrations of SKF-96365 suppressed SMA within 2-3 min and the muscle became quiescent in the absence of pacing (FIG. 32). SKF-96365 displayed an IC₅₀ value of approximately 15 μM.

Therefore, SKF-96365 reverses abnormal automaticity, including SMA.

### Example 5

### SKF-96365 Prevents Abnormal Automaticity (SMA)

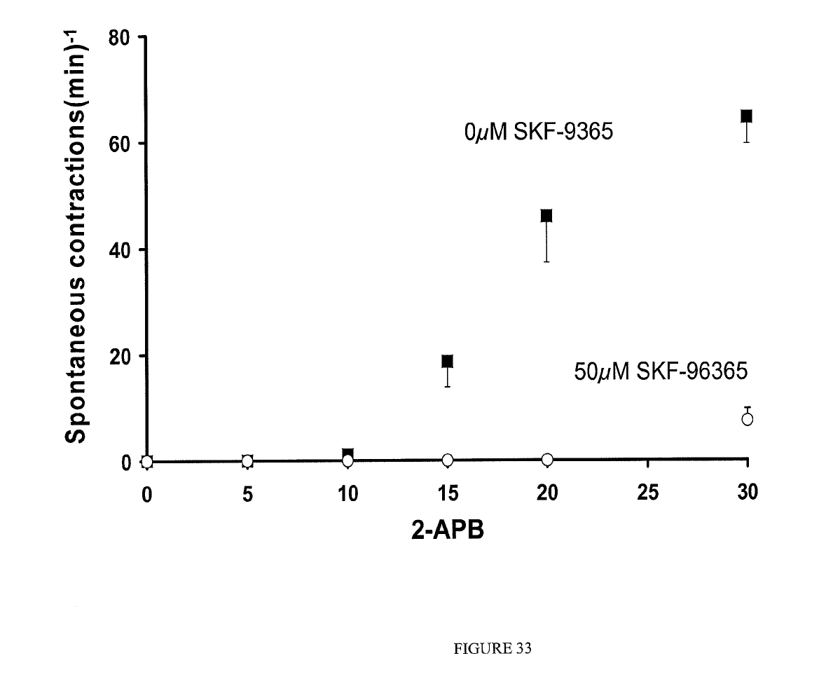

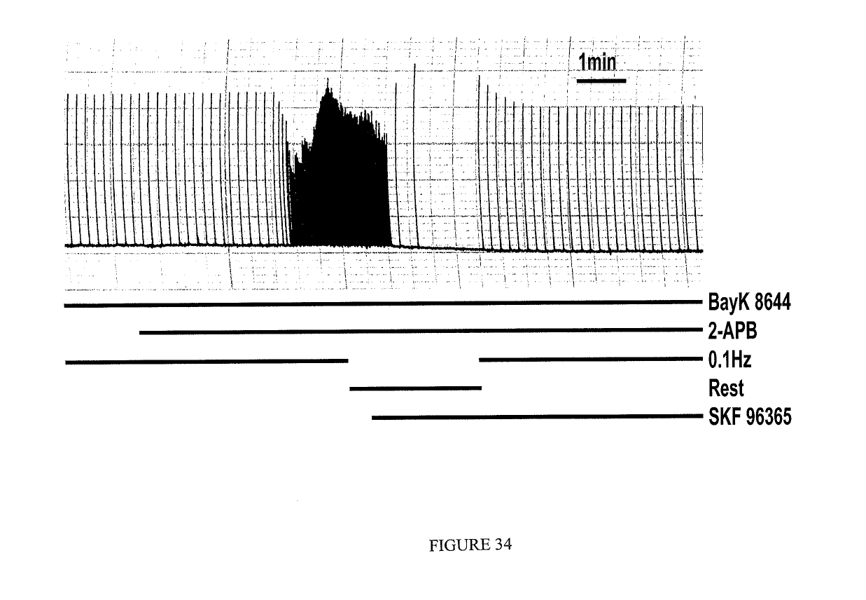

In this example, it was evaluated whether SKF-96365 could prevent abnormal automaticity (SMA). In these experiments, a 2-APB model system was used to simulate abnormal automaticity. Specifically, superfused left atria were exposed to 0 or to 50 μM SKF-96365 for 10 min. Following this pre-incubation, muscles were titrated with increasing concentrations of 2-APB (0-30 μM) and the rate of SMA was measured about 10 min later in the absence of pacing. Pre-treating superfused left atrium with SKF-96365 (50 μM) prevents the appearance of SMA (FIG. 33). In the absence of SKF-96365 pre-treatment, SMA increased in a dose dependent manner.

Therefore, SKF-96365 prevents abnormal automaticity, including SMA.

### Example 6

### SKF-96365 Prevents Tachycardic Abnormal Automaticity

In this example, it was evaluated whether SKF-96365 could reverse tachycardic abnormal automaticity. In these experiments, a 2-APB model system was used to simulate tachycardic abnormal automaticity. Specifically, superfused left atria were treated sequentially with 300 nM BayK 8644, an activator of voltage-dependent calcium entry, which loads myocytes with calcium, and then with 2-APB (20 μM) to activate the putative myocyte system for abnormal automaticity. As expected, the muscle began to exhibit STA in the absence of pacing (FIG. 34, line marked “Rest”). SKF-96365 (10 μM) was added to the superfusate. SKF-96365 reversed STA in these left atria within 2-3 min and the muscle became quiescent in the absence of pacing (FIG. 34, “Rest”). SKF-96365 did not affect normal excitation-contraction coupling, as restoring the 0.1 Hz pacing stimulus provoked mechanical activity similar in magnitude to untreated left atria which occurred only following a pacing stimulus.

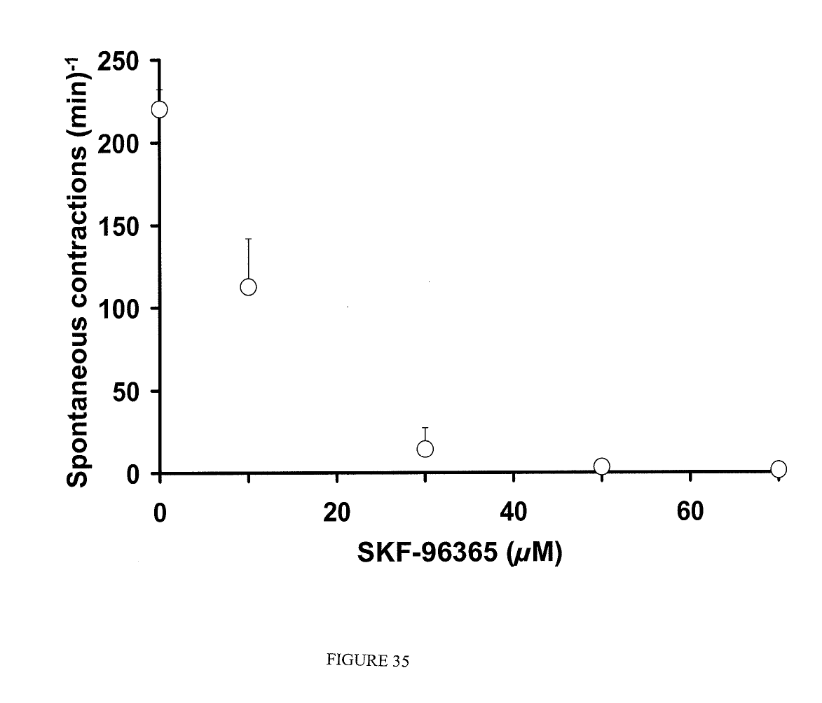

Additional experiments determined the concentration-dependence of SKF-96365 inhibition of STA. These experiments were performed as described above except that various concentrations of SKF-96365 were added to the left atrial superfusate (about 10-70 μM). Exposing left atria to various concentrations of SKF-96365 reversed STA (FIG. 35).

Therefore, SKF-96365 reverses tachycardic abnormal automaticity.

### Example 7

### SKF-96365 Reverses Chaotic, Fibrillation-like Activity

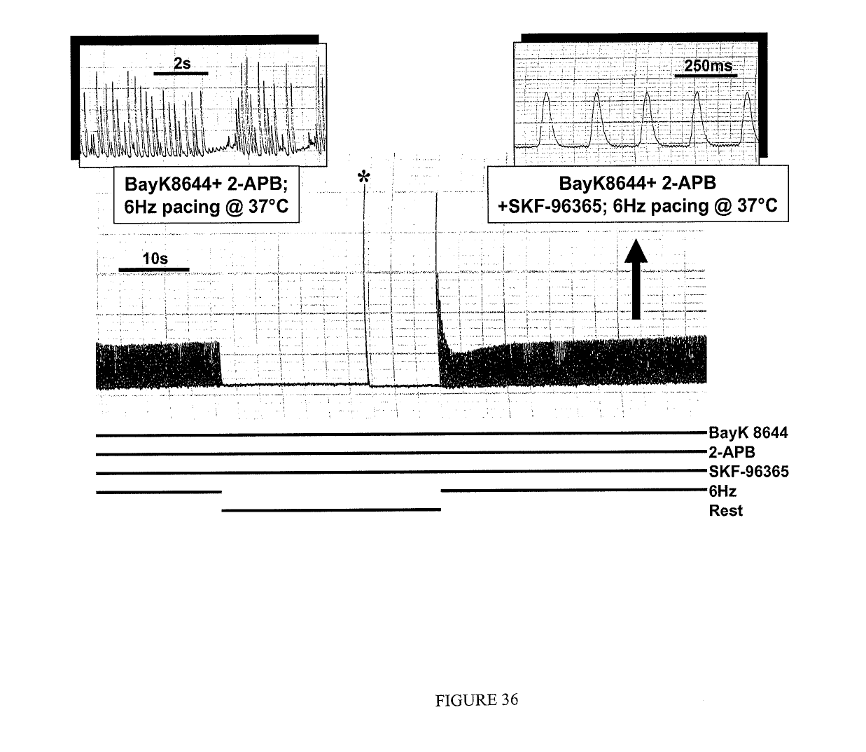

In this example, it was evaluated whether SKF-96365 could reverse chaotic, fibrillation-like activity. In these experiments, left atria were superfused at 37° C., paced at 6 Hz (a physiological rate under these conditions), exposed to 300 nM BayK 8644 to load calcium and then to 20 μM 2-APB to induce tachycardic activity. Under these conditions muscle exhibited chaotic, fibrillation-like activity (FIG. 36). These normally non-automatic left atria now contract spontaneously at ˜500 contractions/min, ˜8-9 Hz, (FIG. 36; line marked “Rest”). When left atria then are paced at a physiological rate (˜6 Hz), these muscles descend into chaotic mechanical activity. Treating these chaotically contracting muscles with SKF-96365 (˜40 μM) restores normal patterns of excitation-contraction after ˜5-8 min. Furthermore, the left atria required external stimulation for mechanical activity (FIG. 36; Lower panel; Rest).

Therefore, SKF-96365 reverses chaotic, fibrillation-like activity.

### CONCLUSION

Therefore, the examples above show that SKF-96365 reverses and prevents triggered activity, reverses and prevents abnormal automaticity, reverses tachycardic abnormal automaticity, and reverses fibrillation-like chaotic activity. These results demonstrate the anti-arrhythmic effects of SKF-96365.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein.

1. A method of treating or preventing an arrhythmia in a subject in need thereof, the method comprising the step of administering to the subject a therapeutically effective amount of a compound of the formula (I), a derivative thereof, a tautomer of any of the foregoing, a polymorphic variant of any of the foregoing or a pharmaceutically acceptable salt of any of the foregoing, wherein the compound of the formula (I) has the following general structure: 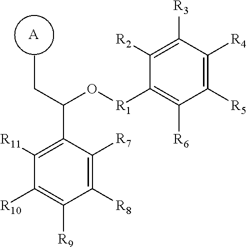
wherein,
R₁ is (CH₂)ₙ, where n is 0 to 10;
R₂ through R₁₁ are each independently selected from the group consisting of: —H, —OH, halogen, alkyl, substituted alkyl, alkenyl, substituted alkenyl, alkynyl, substituted alkynyl, —NH₂, —NR₁₂R₁₃, —COR₁₄, —CON═N═N, —N═NR₁₅, —N═NOR₁₆, —N═NNR₁₇, —SR₁₈, —SOR₁₉, —SO₂R₂₀, —SO₃R₂₁, —OR₂₂ and —XR₂₃;
X is selected from the group consisting of: selenium (Se), tellurium (Te), polonium (Po), and technetium (Tc);
R₁₂, R₁₃, R₁₅, R₁₆ and R₁₇ and R₂₃ are each independently selected from the group consisting of: —H, —OH, alkyl, substituted alkyl, alkenyl, substituted alkenyl, alkynyl, and substituted alkynyl;
R₁₄, R₁₈, R₁₉, R₂₀ and R₂₁ are each independently selected from the group consisting of: —H, —OH, halogen, unsubstituted alkyl, substituted alkyl, alkenyl, substituted alkenyl, alkynyl, substituted alkynyl, and —NR₁₂R₁₃;
R₂₂ is selected from the group consisting of —H, —OH, halogen, unsubstituted alkyl, substituted alkyl, alkenyl, substituted alkenyl, alkynyl, substituted alkynyl —COR₁₄, and —NR₁₂R₁₃; and
A is selected from the group consisting of: a substituted heterocyclyl, an unsubstituted heterocyclyl, an unsubstituted heterocyclylalkyl, and a substituted heterocyclylalkyl.

2-70. (canceled)
